(19) United States
(12) Patent Application Publication
(10) Pub. No.: US 2010/0324086 A1
(43) Pub. Date: Dec. 23, 2010

(54) COMPOUNDS AND METHODS

(75) Inventors: Johan Wannberg, Uppsala (SE); Mathias Alterman, Stockholm (SE); Patric Stenberg, Stockholm (SE); Jacob Westman, Jarlasa (SE)

Correspondence Address: BROWDY AND NEIMARK, P.L.L.C.;624 NINTH STREET, NW, SUITE 300, WASHINGTON, DC, 20001-5303, US

(73) Assignee: Novasaid AB, Stockholm (SE)

(21) Appl. No.: 12/735,822

(22) PCT Filed: Feb. 19, 2009
(86) PCT No.: PCT/EP2009/052003

§ 371 Date: Aug. 19, 2010

Related U.S. Application Data
(60) Provisional application No. 61/029,681, filed on Feb. 19, 2008.

(30) Foreign Application Priority Data
Feb. 19, 2008 (EP) 08151630.4

(51) Int. Cl. A61K 31/472; C07C 237/52; A61K 31/167; A61P 35/00; A61P 29/00; C07D 215/42; A61K 31/4706; C07D 217/02; A61K 31/44; C07D 213/40; C07D 235/30; A61K 31/4184; C07D 277/82; A61K 31/428

(52) U.S. Cl. 514/310; 564/155; 514/616; 546/162; 514/313; 546/143; 514/357; 546/337; 548/307.4; 514/395; 548/163; 514/367

(57) ABSTRACT

A compound of formula (1) as well as pharmaceutically acceptable salts thereof, and a pharmaceutical composition comprising the compound. The compound is useful for the treatment of disorder from pain, fever, inflammation and cancer.

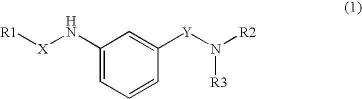

### FIELD OF THE INVENTION

The present invention relates to 1,3-disubstituted phenyl derivatives and to the use thereof in disease therapy. More particularly, the present invention relates to 1,3-disubstituted phenyl derivatives for the treatment of inflammation related diseases. Even more particularly, the present invention relates to compounds acting on microsomal prostaglandin E synthase for the treatment and prevention of fever, pain, cancer, inflammation and inflammatory diseases.

### BACKGROUND OF THE INVENTION

The present invention relates to compounds which inhibit, regulate and/or modulate the activity of microsomal prostaglandin E synthase, compositions which contain these compounds, and methods of using them to treat diseases and conditions such as pain, fever, inflammations and cancer, and the like in mammals.

The following is provided as background information only and should not be taken as an admission that any subject matter discussed or that any reference mentioned is prior art to the instant invention.

Prostaglandin (PG) E₂ is produced in a sequential action including liberation of arachidonic acid, conversion into PGG₂/PGH₂ by cyclooxygenase (Cox)-1 or Cox-2 and finally prostaglandin E synthase converts PGH₂ into PGE₂ (FIG. 1A). There exist three known enzymes that catalyze the latter reaction i.e. microsomal prostaglandin E synthase 1 (MPGES1), cytosolic prostaglandin E synthase (CPGES), and microsomal prostaglandin E synthase 2 (MPGES2). The latter two enzymes are constitutively expressed whereas MPGES1 is inducible by proinflammatory cytokines. Initially, MPGES1 was regarded as the enzyme predominantly coupled with Cox-2 activity. However; later results demonstrate that MPGES1 can also catalyze the conversion of Cox-1 derived PGH2 into PGE2. MPGES1 possesses the highest catalytic efficiency of the known PGE synthases. The role of PGE2 as one of the most potent mediators of inflammation together with many in vitro reports on the presence of MPGES1 in different models of inflammation suggested this enzyme to be an attractive drug target for development of new anti inflammatory drugs with fewer side effects than the currently available NSAIDs and selective Cox-2 inhibitors. The rationale being that MPGES1 is predominantly expressed during inflammation and that other enzymes exist that mediate house keeping functions.

NSAIDs constitute many drugs that inhibit Cox-1 and Cox-2 with a continuum of different potencies on respective enzymes. They range from acetyl salicylic acid, being a preferred Cox-1 inhibitor, to selective Cox-2 inhibitors, e.g. rofecoxib or celecoxib (Vioxx and Celebrex, respectively). Cox-1 inhibitors are cardio-protective by their capability to prevent thromboxane formation in platelets while deleterious vascular effects after prolonged usage of selective Cox-2 inhibitors have been reported, likely through the effect of Cox-2 dependent prostacyclin formation in endothelial cells. The ratio of thromboxane:prostacyclin is diminished by Cox-1 inhibitors but increased by Cox-2 inhibitors. Cox-1 inhibitors are also known to result in increased frequency of gastric bleedings and kidney function impairments. Cox-2 inhibitors also result in gastric side effects as well as negative changes in the body water-salt balance with problems of edema formation and hypertension as a consequence. This seems particularly a problem for rofecoxib.

Specific inhibition of MPGES1 may overcome many of these side effects due to the fact that the balance among the prostaglandins will not primarily be influenced. Thus only the pro-inflammatory pressure during induced PGE2 formation will be targeted. The possibility also exists that an MPGES1 inhibitor will possess enforced anti-inflammatory potential since Cox-2 generated anti-inflammatory prostaglandins such as cyclopentenones may increase due to shunting of PGH2 in macrophages (FIG. 2B). Such shunting will not occur in platelets (there is no evidence for PGE synthase activity in these cells). In endothelial cells there might occur a shunting upon activation since these cells become activated during inflammation which leads to high formation of PGE2 and prostacyclin. In that case, we expect increased prostacyclin formation with protecting effects against vascular side effects.

Although the bulk of evidence already suggested MPGES1 to constitute a drug target, the results using gene targeted knock-out mice have provided unequivocal important data regarding the physiological role of MPGES1. 1) These mice develop significantly less arthritis symptoms in experimental models of arthritis (CIA and AIA). 2) These mice demonstrate less sensitivity to pain both induced in inflammatory settings and neuropathic settings. 3) These mice do not develop endotoxin, IL-1beta or cytokine induced fever. 4) Finally, as of today, gross histopathological examinations of various organs including the GI tract, behavioural and reproductive parameters have not demonstrated any differences to the results obtained for wild type animals. However, in an ongoing study, the healing phase after heart damage caused by permanent ligation of one major coronary vessel in MPGES1 knock-out mice suggest impaired healing or remodeling of the heart. This must be compared with Cox-2 knock-out mice where the heart spontaneously develop fibrosis and cannot be used in such models. Also, in higher mammals, MPGES2 may take over this function since it is predominantly expressed in the heart.

### SUMMARY OF THE INVENTION

There still is a need for new and efficacious drugs for the treatment of conditions such as fever, pain and inflammation or inflammatory conditions, as well as cancer, and one main object of the present invention is to provide such drugs.

Therefore, according to a first aspect, the invention provides a compound of formula (I)

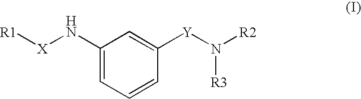

wherein
R¹ is a moiety selected from monocyclic or bicyclic carbocyclyl, monocyclic or bicyclic heterocyclyl, alkyl, and alkenyl, which moiety is optionally substituted with one or more R⁴;
R² and R³ are each independently selected from H and a moiety selected from alkyl, monocyclic or bicyclic carbocyclyl and monocyclic or bicyclic carbocyclyl heterocyclyl, which moiety optionally is substituted with one or more R⁵;
each R⁴ and R⁵ is independently selected from halogen, alkyl, monocyclic or bicyclic carbocyclyl, monocyclic or bicyclic heterocyclyl, alkoxy, CN, CF₃, OH, NO₂, COOH, C(O)Oalkyl, OC(O)alkyl, wherein any cyclyl moiety may be substituted with one or more R⁶, each R⁶ is independently selected from CF₃, halogen, alkyl, CF₃O—, N,N-dialkylamino, phenyl, and saturated monocyclic heterocyclyl,
wherein in any of R¹-R⁶, any cyclyl comprises 1-10 C and any alkyl, alkenyl, and alkoxy comprises 1-6 C;
Y and X are independently selected from >CO and >CH₂;
or a pharmaceutically acceptable salt thereof; for use in the treatment of a disorder selected from fever, pain, cancer, inflammation and inflammatory diseases.

According to a further aspect, the invention provides the use of a compound of formula (I)

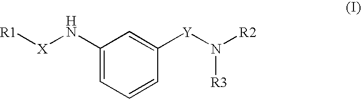

wherein
R¹ is a moiety selected from monocyclic or bicyclic carbocyclyl, monocyclic or bicyclic heterocyclyl, alkyl, and alkenyl, which moiety is optionally substituted with one or more R⁴;
R² and R³ are each independently selected from H and a moiety selected from alkyl, monocyclic or bicyclic carbocyclyl and monocyclic or bicyclic carbocyclyl heterocyclyl which moiety optionally is substituted with one or more R⁵;
each R⁴ and R⁵ is independently selected from halogen, alkyl, monocyclic or bicyclic carbocyclyl, monocyclic or bicyclic heterocyclyl, alkoxy, CN, CF₃CF₃, OH, NO₂NO2, COOH, C(O)Oalkyl, OC(O)alkyl, wherein any cyclyl moiety may be substituted with one or more R⁶,
each R⁶ is independently selected from CF₃CF3, halogen, alkyl, CF₃CF3O—, N,N-dialkylamino, phenyl, and saturated monocyclic heterocyclyl,
wherein in any of R¹-R⁶, any cyclyl comprises 1-10 C and any alkyl, alkenyl, and alkoxy comprises 1-6 C;
Y and X are independently selected from >CO and >CH₂;
or a pharmaceutically acceptable salt thereof; in the manufacturing of a medicament for the treatment of a disorder selected from fever, pain, cancer, inflammation and inflammatory diseases.

According to a further aspect, the invention provides a compound of formula (II)

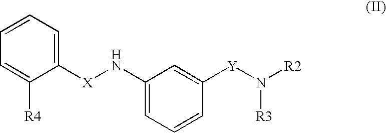

wherein
R² and R³ are independently selected from H and a moiety selected from alkyl, monocyclic or bicyclic carbocyclyl and monocyclic or bicyclic heterocyclyl, which moiety optionally is substituted with one or more R⁵;
R⁴ is selected from halogen, C1-C4-alkyl, e.g. methyl and ethyl, monocyclic C5-C10-carbocyclyl, e.g. C6-carbocyclyl, monocyclic 5 or 6-membered heterocyclyl, e.g. 5-membered heterocyclyl, C1-C4-alkoxy, CF₃, NO₂, COOH, —C(O)O—C1-C4)-alkyl, —OC(O)—C1-C4-alkyl;
R⁵ is selected from halogen, alkyl, monocyclic or bicyclic carbocyclyl, monocyclic or bicyclic heterocyclyl, alkoxy, CN, CF₃, OH, NO₂, COOH, C(O)Oalkyl, OC(O)alkyl, wherein any cyclyl moiety may be substituted with one or more R⁶,
each R⁶ is independently selected from CF₃, halogen, alkyl, CF₃O, o, phenyl, and saturated monocyclic heterocyclyl,
wherein in any of R², R³, R⁵ and R⁶, any cyclyl comprises 1-10 C and any alkyl and alkoxy comprises 1-6 C;
Y and X are independently selected from >CO and >CH₂;
as well as pharmaceutically acceptable salts thereof; for use as a medicament.

According to a further aspect, the invention provides a compound of formula (II)

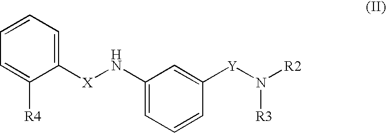

wherein
R² and R³ are independently selected from H and a moiety selected from alkyl, monocyclic or bicyclic carbocyclyl and monocyclic or bicyclic heterocyclyl, which moiety optionally is substituted with one or more R⁵;
R⁴ is CF₃ or chloro;
each R⁵ is independently selected from halogen, alkyl, monocyclic or bicyclic carbocyclyl, monocyclic or bicyclic heterocyclyl, alkoxy, CN, CF₃, OH, NO₂, COOH, C(O)Oalkyl, OC(O)alkyl, wherein any cyclyl moiety may be substituted with one or more R⁶,
each R⁶ is independently selected from CF₃, halogen, alkyl, CF₃O, N,N-dialkylamino, phenyl, and saturated monocyclic heterocyclyl,
wherein in any of R², R³, R⁵ and R⁶, any cyclyl comprises 1-10 C and any alkyl and alkoxy comprises 1-6 C;
Y and X are independently selected from >CO and >CH₂;
as well as pharmaceutically acceptable salts thereof; with the proviso that the compound is not 3-(2-chloro-benzoylamino)-N-naphtalene-1-yl-benzamide, 3-(2-chloro-benzoylamino)-N-(2,6-dimethyl-4-(thiophen-2-yl)phenyl)-benzamide, or 3-(2-chloro-benzoylamino)-N-(2,6-dimethyl-4-(thiophen-2-yl)phenyl)-benzamide.

According to still a further aspect, the present invention provides a compound of formula (II) as defined herein above, or a pharmaceutically acceptable salt thereof, for use in the treatment of a disorder selected from fever, pain and inflammation and cancer.

According to still a further aspect, the present invention provides the use of a compound of formula (II) as defined herein above, or a pharmaceutically acceptable salt thereof, for the manufacturing of a medicament for the treatment of a disorder selected from fever, pain and inflammation and cancer.

According to still a further aspect, the present invention provides a method of treatment of a mammal in need of such treatment, by administering to said mammal a therapeutically effective amount of a compound according to formula (I) or (II), as defined herein above, or a pharmaceutically acceptable salt thereof.

Furthermore, the present invention provides a compound of formula (I) or (II) as defined herein above, or a pharmaceutically acceptable salt thereof, for the treatment or prevention of inflammatory diseases such as rheumatoid arthritis, inflammatory bowel disease, psoriasis, asthma, chronic obstructive pulmonary disease, ulcers, inflammatory pain, fever, atherosclerosis, coronary artery disease, eczema, dermatitis, stroke, diabetes, autoimmune diseases, multiple sclerosis, arthritis or another malignancy as well as cancer, as well as the use of the said compounds in the manufacture of medicaments for the treatment of those disorders.

It is to be noted that compounds of formula (II) correspond to a subgroup of the compounds of formula (I), i.e. those compounds of formula (I) wherein R¹ is phenyl monosubstituted in ortho with R⁴ as defined herein above, in particular with CF₃ or Cl, with the proviso that the novel compounds of formula (II) do not comprise 3-(2-chloro-benzoylamino)-N-naphtalene-1-yl-benzamide, 3-(2-chloro-benzoylamino)-N-(2,6-dimethyl-4-(thiophen-2-yl)phenyl)-benzamide, and 342-chloro-benzoylamino)-N-(2,6-dimethyl-4-(thiophen-2-yl)phen yl)-benzamide.

According to one aspect of the invention, methods for preparing a compound of formula (I) (including a compound of formula (II) are provided.

One method according to the invention, wherein both X and Y are >CO, is as outlined in Reaction Scheme 1:

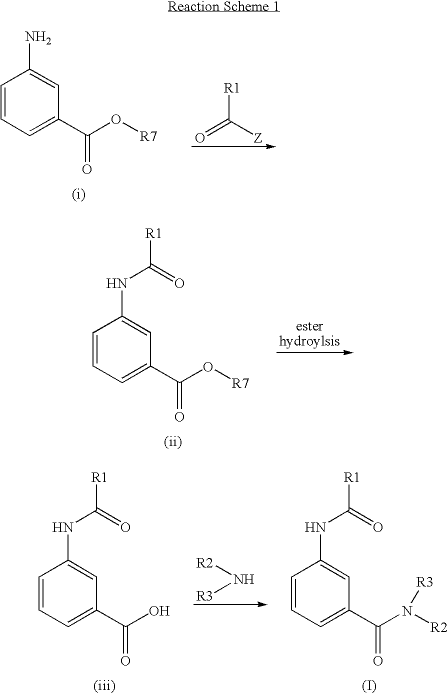

and comprises reacting a 3-aminobenzoic acid ester (i), wherein R⁷ may be e.g. C1-C3-alkyl, with a carboxylic acid (Z is OH) or derivate, e.g. an acid anhydride (e.g. Z is R¹C(O)) or acid chloride (Z is Cl), under conditions suitable for amide coupling; hydrolysing the ester functionality of (ii), e.g. by treatment with NaOH in THF (tetrahydrofuran) to obtain the corresponding acid (iii); and reacting the acid (iii) with an amine R²R³NH under conditions suitable for amide coupling, so as to obtain a compound of formula (I) or (II) as defined herein above.

Conditions suitable for amide coupling will be well-known to the person of ordinary skill in the art, and may be e.g. obtained by performing the reaction of acid and amine in the presence of a suitable amide coupling reagent, such as HATU (o-(7-azabenzotriazol-1-yl)-N,N,N′,N′-tetramethyluronium hexafluorophosphate; CAS Registry Number: 148893-10-1) and N,N-diisopropylethylamine in a suitable solvent, such as CH₂Cl₂, or by reacting the acid derivative in the presence of e.g. triethylamine in a suitable solvent, such as CHCl₃ or in the presence of N,N-diisopropylethylamine in a suitable solvent, such as CH₂Cl₂.

Another method according to the invention, wherein both X and Y are >CO, is as outlined in Reaction Scheme 2:

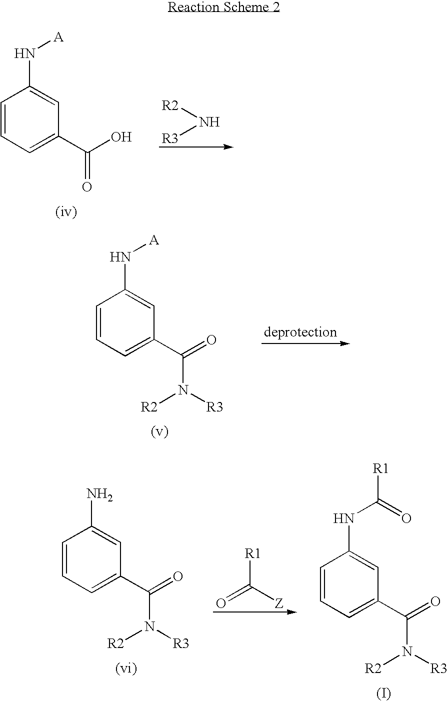

and comprises reacting a 3-aminobenzoic acid (iv), wherein the amino group is protected by a suitable protecting group A, such as tert-butoxy carbonyl (t-Boc), with an amine R²R³NH under conditions suitable for amide coupling, so as to obtain the compound (v); deprotecting the 3-amino function of (v), e.g. by treatment with TFA (trifluoroacetic acid) and reacting the compound (vi) thus obtained with a carboxylic acid or derivative R¹C(O)Z, wherein Z may be e.g. as defined herein above, under conditions suitable for amide coupling, e.g. as mentioned herein above, so as to obtain a compound of formula (I) or formula (II) as defined herein above.

Still another method according to the invention, wherein X is >CO and Y is >CH₂, is as outlined in Reaction Scheme 3:

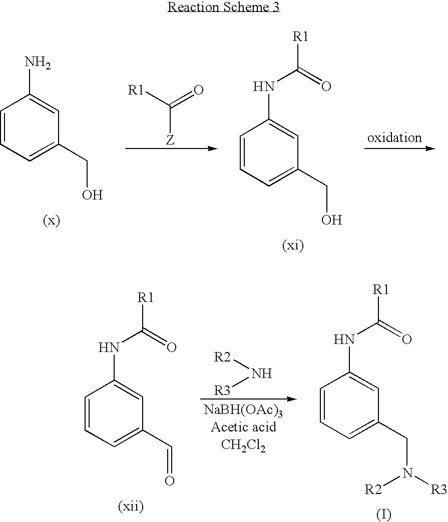

and comprises reacting 3-amino-benzyl alcohol (x) with a carboxylic acid or derivative R¹C(O)Z, wherein Z may be e.g. as defined herein above, under conditions suitable for amide coupling, e.g. as mentioned herein above; oxidizing the alcohol function, e.g. by use of Dess-Martin reagent (1,1,1-Triacetoxy-1,1-dihydro-1,2-benziodoxol-3(1H)-one) followed by work-up with Na₂S₂O₃/NaHCO₃; and reacting the compound (xii) with an amine R²R³NH, e.g. in the presence of acetic acid and NaBH(OAc)₃, to obtain a compound of formula (I) or formula (II) as defined herein above.

A further method according to the invention, wherein X is >CH₂ and Y is >CO, is as outlined in Reaction Scheme 4:

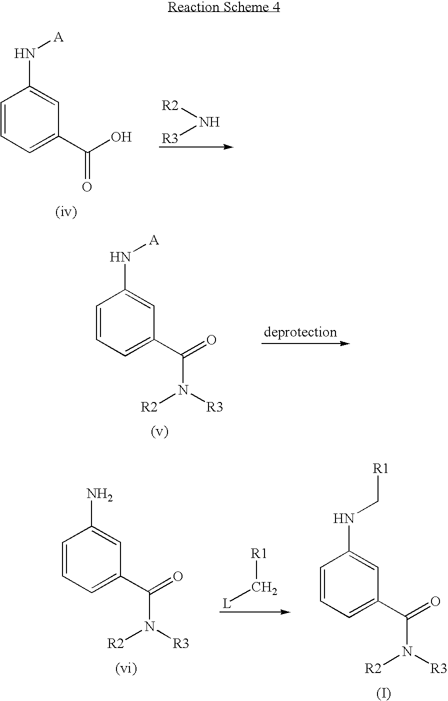

and comprises reacting a 3-aminobenzoic acid (iv), wherein the amino group is protected by a suitable protecting group A, such as tert-butoxy carbonyl (t-Boc), with an amine R²R³NH under conditions suitable for amide coupling, so as to obtain the compound (v); deprotecting the 3-amino function of (v), e.g. by treatment with TFA (trifluoroacetic acid) and reacting the compound (vi) thus obtained with L-CH₂—R¹, wherein L is a suitable leaving group, such as Br, to obtain a compound of formula (I) or formula (II) as defined herein above.

The above methods, as outlined in Reaction Schemes 1-4, are only exemplary and it is contemplated that the skilled person will be able to modify them in view of the particular compound according to formula (I) or formula (II) that is to be prepared, or even to identify other equally suitable methods.

Any further objects, aspects and embodiments of the invention are as defined in the claims.

### BRIEF DESCRIPTION OF THE DRAWINGS

FIG. 1. Biosynthesis of prostaglandin E2. Hypothetic effect of MPGES1 blockage—shunting into the anti-inflammatory PGD2 pathway and formation of cyclopentenons.

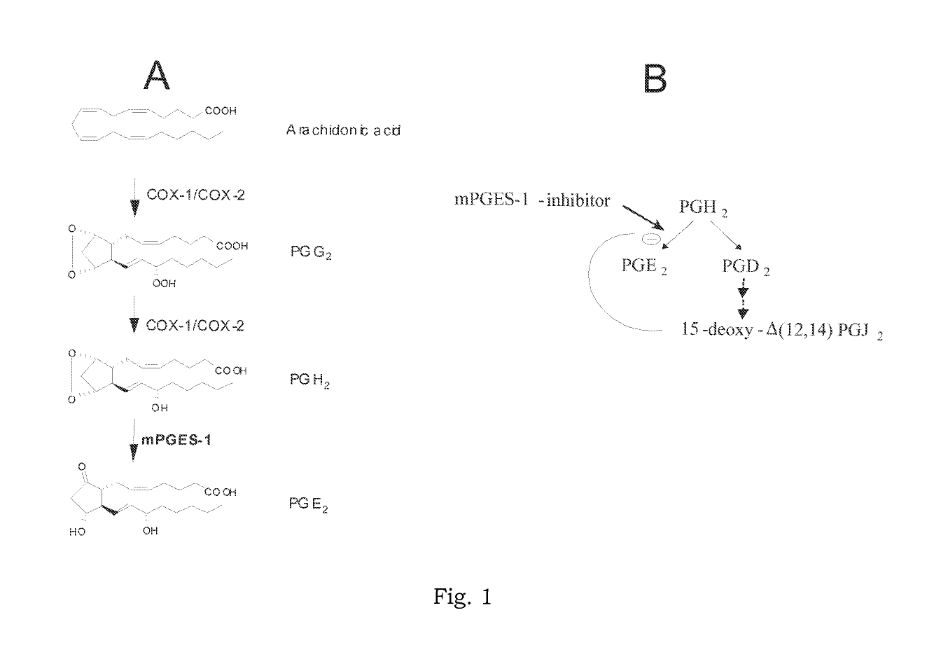

FIG. 2. Schematic representation of biochemical pathway in TBA-MDA Assay.

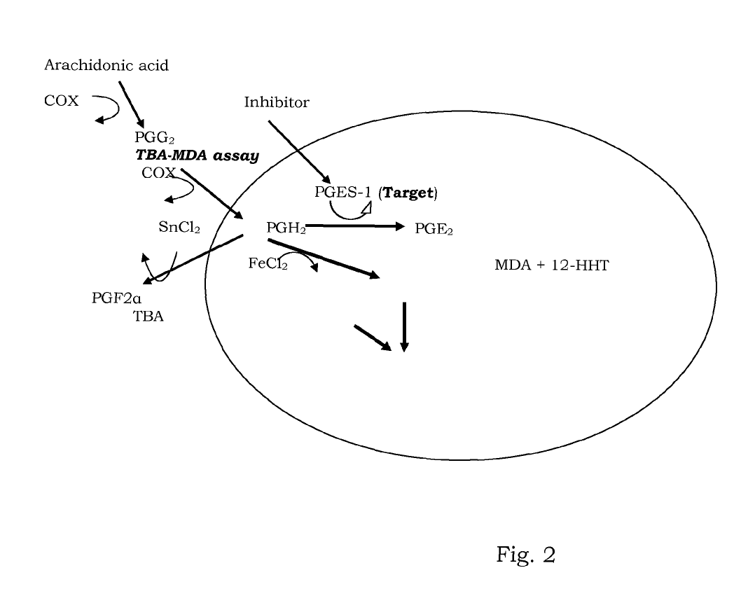

FIG. 3. Net paw swelling (in 0.01 ml) of test animals at number of days after treatment with Complete Freund's Adjuvant (CFA).

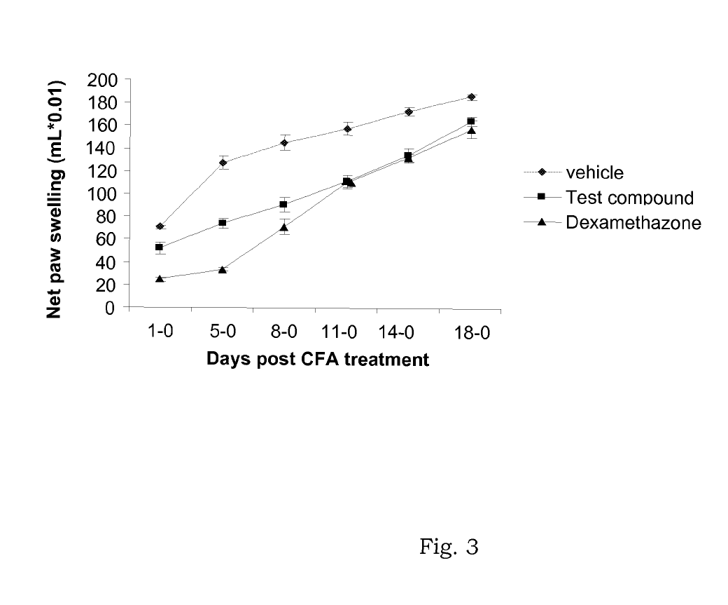

### DETAILED DESCRIPTION OF THE INVENTION

As pointed out herein above, some of the compounds of the invention, as defined by formula (II), are novel or have not hitherto been used in therapy. It should be understood that the following detailed description of the compounds of formula (I) also refers to the compounds of formula (II), in so far as R¹ and R⁴ are as defined with respect to the novel and/or not previously therapeutically used compounds.

Unless otherwise indicated or apparent from the context, any alkyl or alkenyl group as referred to herein may be branched or unbranched. This also applies to said groups when present in moieties such as alkoxy groups etc.

It is contemplated that any branched, linear or cyclic moiety may be attached to another part of the molecule of formula (I) by a bond to any location on the moiety which is available for such binding.

As used herein, the term “carbocyclyl” refers to a cyclic moiety containing only carbon atoms, while the term “heterocyclyl” refers to a cyclic moiety containing not only carbon atoms, but also at least one other atom in the ring structure, e.g. a nitrogen, sulphur or oxygen atom.

As used herein with respect to any carbocyclyl or heterocyclyl, the term monocyclic refers to a cyclic moiety containing only one ring. The term bicyclic refers to a cyclic moiety containing two rings, fused to each other.

Unless otherwise indicated or apparent from the context, any cyclyl, as referred to herein, may be carbocyclyl or heterocyclyl, saturated or unsaturated, and aromatic or non-aromatic.

The term “aromatic”, as used herein, refers to an unsaturated cyclic moiety that has an aromatic character, while the term “non-aromatic”, as used herein, refers to a cyclic moiety, that may be unsaturated, but that does not have an aromatic character.

In a bicyclic ring system, as referred to herein, the rings may be both saturated or both unsaturated, e.g. both aromatic. The rings may also be of different degrees of saturation, and one ring may be aromatic whereas the other is non-aromatic. The rings also may comprise different numbers of atoms, e.g. one ring being 5-membered and the other one being 6-membered.

In a bicyclic heterocyclyl, as referred to herein, one or both of the rings may contain one or several, e.g. 1 or 2, cyclic heteroatoms.

The term “cyclic”, as used herein in respect of an atom, refers to an atom that is a member of at least one ring in a carbocycle or heterocycle. For example, pyridine contains five cyclic C and one cyclic N.

A cyclyl according to the invention may comprise 1-10 C, it being understood that in case the cyclyl comprises less than 3 C, it must necessarily also comprise at least one heteroatom.

By heteroatom according to the invention is meant e.g. N, O and S.

An n-membered cyclic moiety as referred to herein contains n cyclic atoms.

An alkyl moiety according to the invention having from 1-6 C may be selected from methyl, ethyl, n-propyl, i-propyl, n-butyl, i-butyl, tert-butyl, n-pentyl, 2-methylbutyl, 2,2-dimethylpropyl, n-hexyl, 2-methylpentyl, 3-methylpentyl, 2,2-dimethylbutyl, 2,3-methylbutyl.

An alkenyl moiety according to the invention has 2-6 C, e.g. 2-4 C, and comprises at least one double bond between any two adjacent carbon atoms, e.g. one double bond.

A saturated carbocyclyl according to the invention may be monocyclic, e.g. a cycloalkyl having 3-7 C, i.e. cyclopropyl, cyclobutyl, cyclopentyl, cyclohexyl and cycloheptyl, or may be bicyclic.

An aromatic carbocyclyl according to the invention may be selected from phenyl or naphthyl. Unless otherwise indicated or apparent from the context, in a bicyclic moiety according to the invention, each constituent monocycle may independently be selected from aromatic or non-aromatic carbo- and heterocycles.

Examples of aromatic heterocyclic moieties according to the invention are pyridyl, pyrrolyl, quinolinyl, furyl, thienyl, oxadiazolyl, thiadiazolyl, thiazolyl, oxazolyl, pyrazolyl, triazolyl, tetrazolyl, tetrahydroquinolinyl, tetrazolyl, thiadiazolyl, thiazolyl, thiochromanyl, triazolyl, isoxazolyl, isothiazolyl, isoquinolinyl, naphthyridinyl, imidazolyl, pyrimidinyl, phthalazinyl, indolyl, pyrazolyl, pyridazinyl, pyrimidinyl, pyrrolyl, quinazolinyl, quinolinyl, quinolizinyl, quinoxalinyl, tetrahydroisoquinolinyl, pyrazinyl, indazolyl, indolinyl, indolyl, pyrimidinyl, thiophenetyl, pyranyl, chromanyl, cinolinyl, quinolinyl, benzimidazolyl, benzodioxanyl, benzodioxepinyl, benzodioxolyl, benzofuryl, benzothiazolyl, benzoxadiazolyl, benzothiazolyl, benzoxazinyl, benzoxazolyl, benzimidazolyl, benzomorpholinyl, benzoselenadiazolyl, benzothienyl, purinyl, cinnolinyl, pteridinyl and the like.

As used herein, the term “non-aromatic heterocycle” or “non-aromatic heterocyclyl” means a non-aromatic cyclic group containing one or more heteroatom(s) preferably selected from N, O and S, such as a aziridinyl, azetidinyl, dihydropyranyl, dihydropyridyl, dihydropyrrolyl, dioxolanyl, dioxanyl, dithianyl, dithiolanyl, imidazolidinyl, imidazolinyl, morpholinyl, oxetanyl, oxiranyl, pyrrolidinyl, pyrrolidinonyl, piperidyl, piperazinyl, piperidinyl, pyranyl, pyrazolidinyl, quinuclidinyl, sulfalonyl, 3-sulfolenyl, tetrahydrofuryl, tetrahydropyranyl, tetrahydropyridyl, thietanyl, thiiranyl, thiolanyl, thiomorpholinyl, trithianyl, tropanyl, monosaccharide and the like.

As used herein the term “halogen” (or “halo”) means a fluorine, chlorine, bromine or iodine.

According to a first aspect the invention provides a compound according to formula (I), as defined herein above, for use in the treatment of a disorder selected from fever, pain, cancer, inflammation and inflammatory diseases; as well as the use of a compound according to formula (I), for the manufacturing of a medicament for the treatment of a disorder selected from fever, pain, cancer, inflammation and inflammatory diseases.

In the inventive compounds according to formula (I), R¹ is selected from monocyclic or bicyclic carbocyclyl, monocyclic or bicyclic heterocyclyl, alkyl, and alkenyl, which in one embodiment is unsubstituted and in another is substituted with one or more R⁴, e.g 1-3 R⁴, in particular 1 or 2 R⁴.

When R¹ is substituted by more than one R⁴, these may be the same or different, i.e. are selected independently from each other.

In one embodiment of the invention, in a compound according to formula (I), R¹ is selected from alkyl having from 1 to 4 C, e.g. methyl and ethyl; alkenyl having one double-bond and 2-4 C, e.g. ethenyl; monocyclic or bicyclic carbocyclyl having from 3 to 10 C, e.g. cyclopropyl, phenyl and naphthyl; monocyclic 5- or 6-membered heterocyclyl, having at least one heteroatom selected from N, O and S, R¹ being unsubstituted or substituted with one or more R⁴. When R¹ is substituted with one or more R⁴, it preferably is substituted with 1-3 R⁴, more preferably one or two R⁴, in particular one R⁴.

In one embodiment of the invention, R¹ is a moiety selected from phenyl, naphthyl, aromatic 5-6-membered heterocyclyl, cyclopropyl, methyl, ethyl and ethenyl, which moiety is unsubstituted or substituted with one or more R⁴, e.g. 1-3 R⁴, in particular one or two R⁴, preferably one R⁴.

In one embodiment of the invention, R¹ is selected from phenyl, naphthyl, pyridyl, thienyl, furyl, cyclopropyl, methyl, ethyl and ethenyl and is optionally substituted with one or more R⁴.

In one embodiment, R¹ is selected from methyl, benzyl, halogen, thienyl, furyl, pyridyl, cyclopropyl, diphenylmethyl, cyclopentylethyl, phenylethenyl, naphthyl, phenyl and phenyl substituted with one or several R⁴, e.g. 1-3 R⁴, selected from halogen, methoxy, trifluoromethyl, thienyl, phenyl, ethyl, nitro, acetoxy, methoxycarbonyl and carboxylic acid.

In one embodiment, R¹ is selected from halogen, methyl, benzyl, diphenylmethyl, cyclopentylethyl, E-phenylethenyl, cyclopropyl, phenyl, 3,4-dichlorophenyl, 2-fluorophenyl, 2-chlorophenyl, 3-chlorophenyl, 4-chlorophenyl, 2-bromophenyl, 2-iodophenyl, 2-methylphenyl, 2-ethylphenyl, 2-methoxyphenyl, 4-methoxyphenyl, 2-trifluoromethylphenyl, 4-trifluoromethylphenyl, 3,5-bis(trifluoromethyl)phenyl, 2,6-dichlorophenyl, 2,6-dimethoxyphenyl, 3,4,5-trimethoxyphenyl, 2-thien-2-ylphenyl, 2-acetyloxyphenyl, 4-(1,1′-biphenyl), 2-nitrophenyl, 2-methoxycarbonylphenyl, 2-carboxyphenyl, 1-naphthyl, 2-naphthyl, 2-thienyl, 2-furyl, 3-pyridyl and 4-pyridyl.

In one embodiment, R¹ is phenyl substituted with R⁴ in ortho-position relative to the bond linking the phenyl ring to the carbonyl, i.e. R¹ is phenyl substituted with R⁴ in 2-position.

In another embodiment, R¹ is phenyl substituted with R⁴ in ortho-position relative to the bond linking the phenyl ring to the carbonyl, and optionally substituted with one or more further, independently selected R⁴ in other positions on the ring, e.g. 1 or 2 further R⁴, in particular 1 further R⁴. For example, R¹ may be phenyl substituted in ortho-position with a moiety R⁴ selected from halogen, C1-C4-alkyl, e.g. methyl and ethyl, monocyclic 5 or 6-membered heterocyclyl, e.g. 5-membered heterocyclyl, C1-C4-alkoxy, CF₃, NO₂, COOH, —C(O)O—C1-C4)-alkyl, and —OC(O)—C1-C4-alkyl, and optionally also substituted with one or more, e.g. 1-2, independently selected R⁴ in other ring positions (including the other ortho-position). In this case the R⁴ in ortho-position suitably is selected from CH₃O, CF₃, OC(O)CH₃, NO₂, C(O)OCH₃, COOH, F, Cl, Br; I, methyl, ethyl, and thienyl, e.g. thien-2-yl, in particular from CH₃O, CF₃, F, Cl, Br; I, methyl, ethyl, and thienyl, e.g. thien-2-yl, preferably from halogen and CF₃, more preferably from Cl and CF₃, and even more preferably is CF₃.

In one embodiment, R¹ is phenyl, monosubstituted with R⁴, as defined herein above, in ortho-position relative to the bond linking the phenyl ring to the carbonyl, i.e. phenyl substituted with R⁴ in 2-position. For example, R¹ may be phenyl substituted in ortho-position with a moiety R⁴ selected from halogen, C1-C4-alkyl, e.g. methyl and ethyl, monocyclic 5 or 6-membered heterocyclyl, e.g. 5-membered heterocyclyl, C1-C4-alkoxy, CF₃, NO₂, COOH, —C(O)O—C1-C4-alkyl, and —OC(O)—C1-C4-alkyl. In this case R⁴ suitably is selected from CH₃O, CF₃, OC(O)CH₃, NO₂, C(O)OCH₃, COOH, F, Cl, Br; I, methyl, ethyl, and thienyl, e.g. thien-2-yl, in particular from CH₃O, CF₃, F, Cl, Br; I, methyl, ethyl, and thienyl, e.g. thien-2-yl, preferably from halogen and CF₃, more preferably from Cl and CF₃, and even more preferably is CF₃.

In still another embodiment, R¹ is bicyclic carbocyclyl, e.g. naphthyl.

In one embodiment, R¹ is selected from monocyclic or bicyclic heterocyclyl, as defined herein above.

When R¹ is alkyl or alkenyl, it preferably is substituted with at least one R⁴, e.g. one or two R⁴. In this case, each R⁴ preferably is selected from monocyclic or bicyclic carbocyclyl, and monocyclic or bicyclic heterocyclyl, e.g. from monocyclic carbocyclyl or heterocyclyl, such as C5 or C7 monocyclic carbocyclyl or 5- or 6-membered heterocyclyl.

In the inventive compounds according to formula (I), R² and R³ are each independently selected from H and a moiety selected from alkyl, monocyclic or bicyclic carbocyclyl and monocyclic or bicyclic heterocyclyl, which moiety optionally is substituted with one or more R⁵.

In one embodiment, R² and/or R³ is not substituted with any R⁵, or is substituted by only one R⁵. When R² and/or R³ is a moiety substituted with more than one R⁵, said moiety suitably is substituted with two or three R⁵, preferably two R⁵.

The selection and number of R⁵ on R² and R³, respectively, are independent from each other; e.g. R³ may be unsubstituted while R² is mono- di- or trisubstituted, or is unsubstituted too. Also, when R² and/or R³ is substituted with more than one R⁵, the R⁵ moieties may be the same or different, i.e. are selected independently from each other.

In one embodiment of the invention, R² and R³ are each independently selected from H and a moiety selected from C1-C6-alkyl, e.g. C1-C4-alkyl, and carbocyclyl or heterocyclyl, wherein the cyclyl is monocyclic, e.g. wherein any ring is 5-, 6- or 7-membered, or bicyclic, e.g. a 5- or 6-membered cycle fused with a 6-membered cycle, e.g. benzene, which moiety optionally is substituted with one or more R⁵.

In one embodiment of the invention, R² and R³ are each independently selected from H and a moiety selected from C1-C6-alkyl, e.g. C1-C4-alkyl, and carbocyclyl or heterocyclyl, wherein the cyclyl is monocyclic, e.g. wherein any ring is 5-, 6- or 7-membered, or bicyclic, e.g. a 5- or 6-membered cycle fused with a 6-membered cycle, e.g. benzene, which moiety optionally is substituted with one or more R⁵.

When either of R² and R³ is C1-C6-alkyl or C1-C4-alkyl, it preferably is substituted with at least one R⁵, e.g. at least one R⁵ attached to the carbon atom linking R² or R³ to the benzamide-N (i.e. in 1-position). For example, R² and/or R³ may be methyl carrying at least one R⁵, or ethyl substituted with R⁵ in 1-position.

In one suitable embodiment, when R² and/or R³ is C1-C6-alkyl or C1-C4-alkyl substituted with at least one R⁵, e.g. methyl or ethyl substituted with at least one R⁵, each R⁵ is independently selected from carbocyclyl or heterocyclyl, wherein the cyclyl is monocyclic, e.g. a 5-, 6- or 7-membered ring, such as phenyl or pyridinyl, or bicyclic, e.g. a 5- or 6-membered cycle fused with a 6-membered cycle, such as a 5- or 6-membered cycle fused with phenyl.

When either of R² and R³ is monocycle carbocyclyl, the cyclyl may be saturated, such as cyclohexyl or cycloheptyl, or unsaturated, e.g. aromatic, i.e. phenyl.

When either of R² and R³ is bicyclic carbocyclyl, the degree of saturation of each cycle may differ, such as in indanyl and 1,2,3,4-tetrahydronaphthyl, or be the same, such as in naphthyl.

When either of R² and R³ is monocycle heterocyclyl, this may suitably be a 5-, 6- or 7-membered ring containing 1 heteroatom in the ring, e.g. pyridinyl.

When either of R² and R³ is bicyclic heterocyclyl, this may suitably be a 5- or 6-membered heterocycle containing 1 or 2 heteroatoms in the ring, fused with a 6-membered carbocycle, such as a 5- or 6-membered heterocycle fused with phenyl, e.g. quinolyl, isoquinolyl, 1H-indazolyl, 1H-benzimidazolyl, 1,3-benzothiazolyl, 1,3-benzoxazolyl.

When either or R² and R³ is monocyclic or bicyclic carbocyclyl or monocyclic or bicyclic heterocyclyl carrying at least one R⁵, e.g. 1 or 2 R⁵, each R⁵ suitably is independently selected from fluoro, chloro, bromo, iodo, trifluoromethyl, methyl, cyano, thienyl, phenyl, (dimethylamino)phenyl and hydroxy.

In one embodiment, R² and R³ are each independently selected from H and a moiety selected from methyl, ethyl, n-butyl, cyclohexyl, cycloheptyl, phenyl, pyridyl, quinolinyl, isoquinolinyl, benzimidazolyl, benzothiazolyl, benzoxazol, 1H-indazolyl, indanyl and 1,2,3,4-tetrahydronaphthyl, which moiety optionally is substituted with one or more R⁵.

In one embodiment, R² and R³ are each independently selected from H and a moiety selected from C1-C6-alkyl, C3-C8-cycloalkyl, phenyl, 2-halophenyl, benzyl, 2-trifluoromethylbenzyl, halobenzyl, 2-phenylbenzyl, 3-phenylbenzyl, 3-halobenzyl, 2,3-di(C1-C6-alkyl)benzyl, 2-(C1-C6 alkyl)benzyl, 2-piperidin-1-ylbenzyl, 2-trifluoromethoxybenzyl, 2-alkoxybenzyl, 2-(di-(C1-C6 alkyl))aminobenzyl, 2-(morpholin-4-yl)benzyl, diphenylmethyl, pyridin-2-ylmethyl, pyridin-3-ylmethyl, 1-naphthylmethyl, 2,3-dihydro-1,4-benzodioxin-5-ylmethyl, (1R)-1-(1-naphthyl)ethyl, (1S)-1-(1-naphthyl)ethyl, indan-1-yl, 1,2,3,4-tetrahydronaphth-1-yl, 3-halopyridin-2-yl, pyridin-2-yl, 5-trifluoromethylpyridin-2-yl, 5-(C1-C6-alkyl)pyridin-2-yl, 5-cyanopyridin-2-yl, 5-halopyridin-2-yl, 5-halo-6-(C1-C6-alkyl)pyridin-2-yl, 5-thien-3-ylpyridin-2-yl, 5-phenylpyridin-2-yl, and 5-[4-(di-(C1-C6 alkyl)amino)phenyl]pyridin-2-yl, quinolin-2-yl, 8-hydroxyquinolin-2-yl, 2-(C1-C6-alkyl)quinolin-4-yl, 1H-benzimidazol-2-yl, 1H-indazol-5-yl, 1,3-benzothiazol-6-yl, 1,3-benzothiazol-5-yl, 2-(C1-C6-alkyl)-1,3-benzothiazol-6-yl, 2-(C1-C6-alkyl)-1,3-benzoxazol-6-yl, and 5-halo-1,3-benzoxazol-2-yl.

In one embodiment, R² and R³ are each independently selected from H and a moiety selected from n-butyl, cyclohexyl, cycloheptyl, phenyl, 2-chlorophenyl, benzyl, 2-trifluoromethylbenzyl, 2-bromobenzyl, 2-phenylbenzyl, 3-phenylbenzyl, 3-bromobenzyl, 2,3-dimethylbenzyl, 2-methylbenzyl, 2-piperidin-1-ylbenzyl, 2-trifluoromethoxybenzyl, 2-methoxybenzyl, 2-(dimethylamino)benzyl, 2-chlorobenzyl, 2-(morpholin-4-yl)benzyl, diphenylmethyl, pyridin-2-ylmethyl, pyridin-3-ylmethyl, 1-naphthylmethyl, 2,3-dihydro-1,4-benzodioxin-5-ylmethyl, (1R)-1-(1-naphthyl)ethyl, (1S)-1-(1-naphthyl)ethyl, indan-1-yl, 1,2,3,4-tetrahydronaphth-1-yl, 3-bromopyridin-2-yl, pyridin-2-yl, 5-trifluoromethylpyridin-2-yl, 5-methylpyridin-2-yl, 5-cyanopyridin-2-yl, 5-bromopyridin-2-yl, 5-chloropyridin-2-yl, 5-iodopyridin-2-yl, 5-fluoropyridin-2-yl, 5-bromo-6-methylpyridin-2-yl, 5-thien-3-ylpyridin-2-yl, 5-phenylpyridin-2-yl, and 5-[4-(dimethylamino)phenyl]pyridin-2-yl, quinolin-2-yl, quinolin-8-yl, 2-methylquinolin-4-yl, 8-hydroxyquinolin-2-yl, isoquinolin-5-yl, benzimidazol-2-yl, 1H-indazol-5-yl, 1,3-benzothiazol-6-yl, 1,3-benzothiazol-5-yl, 2-methyl-1,3-benzothiazol-6-yl, 2-methyl-1,3-benzoxazol-6-yl, and 5-chloro-1,3-benzoxazol-2-yl.

In one preferable embodiment R³ is selected from H and a moiety selected from alkyl and monocyclic carbocyclyl, which moiety optionally is substituted with one or more R⁵. In particular, R³ is selected from H; and a moiety selected from alkyl and monocyclic carbocyclyl, which moiety optionally is substituted with one or more R⁵ selected from monocyclic carbocyclyl. For example, R³ may be selected from H; C1-C3 alkyl, e.g. methyl, optionally substituted by monocyclic C5-C7 carbocyclyl, e.g. C6 carbocyclyl; and monocyclic C5-C7 carbocyclyl, e.g. C6 carbocyclyl. In particular R³ may be selected from H, methyl, benzyl and cyclohexyl. Most preferably R³ is H.

In the inventive compounds according to formula (I), each R⁴ and R⁵ is independently selected from halogen, alkyl, monocyclic or bicyclic carbocyclyl, monocyclic or bicyclic heterocyclyl, alkoxy, CN, CF₃, OH, NO₂, COOH, C(O)Oalkyl, OC(O)alkyl, wherein any cyclyl moiety may be substituted with one or more R⁶, e.g. 1-3 R⁶, or 1 or 2 R⁶.

In one embodiment, R⁴ and R⁵ are independently selected from halogen, C1-C4-alkyl, e.g. methyl and ethyl, monocyclic or bicyclic C5-C10-carbocyclyl, monocyclic 5 or 6-membered heterocyclyl or bicyclic heterocyclyl, e.g. containing two 5- or 6-membered rings fused to each other, C1-C4-alkoxy, e.g. CH₃O, CN, CF₃, OH, NO₂, COOH, —C(O)O—C1-C4-alkyl, e.g. —C(O)O—CH₃, —OC(O)C1-C4-alkyl, e.g. —OC(O)CH₃, wherein any cyclyl moiety may be substituted with one or more R⁶.

In one embodiment, R⁴ is selected from halogen, C1-C4-alkyl, e.g. methyl and ethyl, monocyclic C5-C10-carbocyclyl, e.g. C6-carbocyclyl, monocyclic 5 or 6-membered heterocyclyl, e.g. 5-membered heterocyclyl, C1-C4-alkoxy, CF₃, NO₂, COOH, —C(O)O—C1-C4)-alkyl, —OC(O)—C1-C4-alkyl.

For example, R⁴ may be selected from CH₃O, CF₃, OC(O)CH₃, NO₂, C(O)OCH₃, COOH, F, Cl, Br; I, methyl, ethyl, cyclopentyl, phenyl and thienyl, e.g. thien-2-yl.

In one embodiment, each R⁵ is independently selected from halogen, C1-C4-alkyl, monocyclic or bicyclic C5-C10-carbocyclyl, monocyclic 5 or 6-membered heterocyclyl or bicyclic heterocyclyl, e.g. heterocyclyl containing two 5- or 6-membered rings fused to each other, CN, CF₃, OH, wherein any cyclyl moiety may be substituted with one or more R⁶.

When R⁵ is selected from C1-C4-alkyl, it suitably may be selected from C1-C3 alkyl, or C1-C2 alkyl, e.g. methyl.

When R⁵ is monocyclic or bicyclic C5-C10-carbocyclyl, it may be saturated or unsaturated, e.g. aromatic. For example, R⁵ may be selected from phenyl and naphthyl. When R⁵ is phenyl it optionally is substituted with at least one R⁶, e.g. it is phenyl monosubstituted with R⁶. In particular, R⁵ may be phenyl substituted with at least one R⁶ in ortho-position. For example, R⁵ may be phenyl substituted with R⁶ in ortho-position and 1-2 further, independently selected moieties R⁶ in any other position. In one particular embodiment, R⁵ is phenyl monosubstituted with R⁶ in ortho-position.

In the embodiment R⁵ is monocyclic 5 or 6-membered heterocyclyl or bicyclic heterocyclyl, e.g. heterocyclyl containing two 5- or 6-membered rings fused to each other. For example, R⁵ may be monocyclic 5 or 6-membered heterocyclyl. In one embodiment, R⁵ is monocyclic 5 or 6-membered heterocyclyl containing one heteroatom in the ring.

In one embodiment, each R⁵ is independently selected from cyano, CF₃, Br, F, Cl, I, OH, methyl, and a cyclyl selected from phenyl, naphthyl, thienyl and pyridine, wherein any cyclyl, e.g. phenyl or pyridyl, may be substituted with one or more R⁶.

Each R⁶ is independently selected from CF₃, halogen, alkyl, alkoxy, CF₃O, N,N-dialkylamino, phenyl, and saturated monocyclic heterocyclyl.

In one embodiment, each R⁶ is independently selected from CF₃, halogen, C1-C4-alkyl, e.g. methyl, C1-C4-alkoxy, e.g. methoxy, CF₃O, N,N-di-(C1-C4-alkyl)amino, e.g. (CH₃)₂N, phenyl, and saturated monocyclic 6-membered heterocyclyl, e.g. piperidyl and morpholinyl.

As noted herein above, X and Y are independently selected from >CO and >CH₂. In one embodiment, both X and Y are >CO. In another embodiment, X is >CO and Y is >CH₂. In still another embodiment, X is >CH₂ and Y is >CO.

In one particular embodiment, the compound of formula (I) is selected from
3-(2-chloro-benzoylamino)-N-benzyl-benzamide,
3-(2-chloro-benzoylamino)-N-(2-methyl-quinolin-4-yl)-benzamide,
3-(2-chloro-benzoylamino)-N-butyl-benzamide,
3-(2-chloro-benzoylamino)-N,N-dibenzyl-benzamide,
3-(2-chloro-benzoylamino)-N-(diphenylyl-methyl)-benzamide,
3-(2-chloro-benzoylamino)-N-(isoquinolin-5-yl)-benzamide,
3-(2-chloro-benzoylamino)-N-(quinolin-8-yl-)-benzamide,
3-(2-chloro-benzoylamino)-N-(quinolin-2-yl)-benzamide,
3-(2-chloro-benzoylamino)-N-(pyridine-2-ylmethyl)-benzamide,
3-(2-chloro-benzoylamino)-N-(pyridine-3-ylmethyl)-benzamide,
3-(2-chloro-benzoylamino)-N-(pyridine-2-yl)-benzamide,
3-(2-chloro-benzoylamino)-N-(1H-benzoimidazol-2-yl)-benzamide,
3-(2-chloro-benzoylamino)-N-(benzothiazol-6-yl)-benzamide,
3-(2-Chloro-benzoylamino)-N-(1H-indazol-5-yl)-benzamide,
3-benzoylamino-N-quinolin-2-yl-benzamide,
3-acetylamino-N-quinolin-2-yl-benzamide,
thiophene-2-carboxylic acid [3-(quinolin-2-ylcarbamoyl)-phenyl]-amide,
furan-2-carboxylic acid [3-(quinolin-2-ylcarbamoyl)-phenyl]-amide,
N-[3-(quinolin-2-ylcarbamoyl)-phenyl]-nicotinamide,
N-[3-(quinolin-2-ylcarbamoyl)-phenyl]-isonicotinamide,
3-phenylacetylamino-N-quinolin-2-yl-benzamide,
3-(cyclopropanecarbonyl-amino)-N-quinolin-2-yl-benzamide,
3-diphenylacetylamino-N-quinolin-2-yl-benzamide,
3-(3-cyclopentyl-propionylamino)-N-quinolin-2-yl-benzamide,
3-[(E)-(3-phenyl-acryloyl)amino]-N-quinolin-2-yl-benzamide,
3,4-dichloro-N-[3-(quinolin-2-ylcarbamoyl)-phenyl]-benzamide,
3-(3-chloro-benzoylamino)-N-quinolin-2-yl-benzamide,
3-(4-methoxy-benzoylamino)-N-quinolin-2-yl-benzamide,
3-(4-chloro-benzoylamino)-N-quinolin-2-yl-benzamide,
3-(3-trifluoromethyl-benzoylamino)-N-quinolin-2-yl-benzamide,
3-(2-chloro-benzoylamino)-N,N-dicyclohexyl-benzamide,
3-(2-chloro-benzoylamino)-N-indan-1-yl-benzamide,
3-(2-chloro-benzoylamino)-N-(1,2,3,4-tetrahydro-naphthalen-1-yl)-benzamide,
3-(2-chloro-benzoylamino)-N-cycloheptyl-benzamide,
3-(3,5-bis-trifluoromethyl-benzoylamino)-N-quinolin-2-yl-benzamide,
3-(2-bromo-benzoylamino)-N-quinolin-2-yl-benzamide,
3-(2-fluoro-benzoylamino)-N-quinolin-2-yl-benzamide,
3-(2,6-chloro-benzoylamino)-N-quinolin-2-yl-benzamide,
3-(2-methoxy-benzoylamino)-N-quinolin-2-yl-benzamide,
3-(2,6-dimethoxy-benzoylamino)-N-quinolin-2-yl-benzamide,
3-(2-trifluoromethyl-benzoylamino)-N-quinolin-2-yl-benzamide,
3-(2-methyl-benzoylamino)-N-quinolin-2-yl-benzamide,
3-(3,4,5-trimethoxy-benzoylamino)-N-quinolin-2-yl-benzamide,
3-(2-iodo-benzoylamino)-N-quinolin-2-yl-benzamide,
naphthalene-1-carboxylic acid [3-(quinolin-2-ylcarbamoyl)-phenyl]amide,
naphthalene-2-carboxylic acid [3-(quinolin-2-ylcarbamoyl)-phenyl]amide,
3-(2-thiophene-2-yl-benzoylamino)-N-quinolin-2-yl-benzamide,
acetic acid 2-[3-(quinolin-2-ylcarbamoyl)-phenylcarbamoyl]-phenyl ester,
biphenyl-4-carboxylic acid [3-(quinolin-2-ylcarbamoyl)-phenyl]-amide,
3-(2-ethyl-benzoylamino)-N-quinolin-2-yl-benzamide,
3-(2-nitro-benzoylamino)-N-quinolin-2-yl-benzamide,
N-(3-cycloheptylcarbamoyl-phenyl)-3,5-bis-trifluoromethyl-2,6-dichloro-N-(3-cycloheptylcarbamoyl-phenyl)-benzamide,
N-(3-cycloheptylcarbamoyl-phenyl)-2-trifluoromethylbenzamide,
N-(3-cycloheptylcarbamoyl-phenyl)-2-methylbenzamide,
N-(3-cycloheptylcarbamoyl-phenyl)-3,4,5-trimethoxy-benzamide,
naphthalene-1-carboxylic acid (3-cycloheptylcarbamoyl-phenyl)-amide,
naphthalene-2-carboxylic acid (3-cycloheptylcarbamoyl-phenyl)-amide,
biphenyl-4-carboxylic acid (3-cycloheptylcarbamoyl-phenyl)-amide,
N-(3-cycloheptylcarbamoyl-phenyl)-2-thiophene-2-yl-benzamide,
N-(3-cycloheptylcarbamoyl-phenyl)-2-fluorobenzamide,
N-(3-cycloheptylcarbamoyl-phenyl)-2-methoxy-benzamide,
N-(3-cycloheptylcarbamoyl-phenyl)-2,6-dimethoxy-benzamide,
acetic acid 2-(3-cycloheptylcarbamoyl-phenylcarbamoyl)-phenyl ester,
N-(3-cycloheptylcarbamoyl-phenyl)-phthalamic acid methyl ester,
2-bromo-N-(3-cycloheptylcarbamoyl-phenyl)-benzamide,
N-(3-cycloheptylcarbamoyl-phenyl)-2-iodo-benzamide,
N-(3-cycloheptylcarbamoyl-phenyl)-phthalamic acid
N-[3-((S)-1-naphthalen-1-yl-ethylcarbamoyl)-phenyl]-3,5-bis-trifluoromethyl-benzamide,
2-bromo-N-[3-((S)-1-naphthalen-1-yl-ethylcarbamoyl)-phenyl]-benzamide,
2-fluoro-N-[3-((S)-1-naphthalen-1-yl-ethylcarbamoyl)-phenyl]-benzamide,
2,6-dichloro-N-[3-((S)-1-naphthalen-1-yl-ethylcarbamoyl)-phenyl]-benzamide,
2-methoxy-N-[3-((S)-1-naphthalen-1-yl-ethylcarbamoyl)-phenyl]-benzamide,
2,6-dimethoxy-N-[3-((S)-1-naphthalen-1-yl-ethylcarbamoyl)-phenyl]-benzamide,
N-[3-((S)-1-naphthalen-1-yl-ethylcarbamoyl)-phenyl]-2-trifluoromethyl-benzamide,
2-methyl-N-[3-((S)-1-naphthalen-1-yl-ethylcarbamoyl)-phenyl]-benzamide,
2-iodo-N-[3-((S)-1-naphthalen-1-yl-ethylcarbamoyl)-phenyl]-benzamide,
naphthalene-1-carboxylic acid [3-((S)-1-naphthalen-1-yl-ethylcarbamoyl)-phenyl]-amide,
naphthalene-2-carboxylic acid [3-((S)-1-naphthalen-1-yl-ethylcarbamoyl)-phenyl]-amide,
biphenyl-4-carboxylic acid [3-((S)-1-naphthalen-1-yl-ethylcarbamoyl)-phenyl]-amide,
N-[3-((S)-1-naphthalen-1-yl-ethylcarbamoyl)-phenyl]-2-thiophen-2-yl-benzamide,
3,4,5-trimethoxy-N-[3-((S)-1-naphthalen-1-yl-ethylcarbamoyl)-phenyl]-benzamide,
2-chloro-N-[3-((S)-1-naphthalen-1-yl-ethylcarbamoyl)-phenyl]-benzamide,
N-[3-((R)-1-naphthalen-1-yl-ethylcarbamoyl)-phenyl]-3,5-bis-trifluoromethyl-benzamide,
2-bromo-N-[3-((R)-1-naphthalen-1-yl-ethylcarbamoyl)-phenyl]-benzamide,
2-fluoro-N-[3-((R)-1-naphthalen-1-yl-ethylcarbamoyl)-phenyl]-benzamide,
2,6-dichloro-N-[3-((R)-1-naphthalen-1-yl-ethylcarbamoyl)-phenyl]-benzamide,
acetic acid 2-[3-((R)-1-naphthalen-1-yl-ethylcarbamoyl)-phenylcarbamoyl]-phenyl ester,
2-methoxy-N-[3-((R)-1-naphthalen-1-yl-ethylcarbamoyl)-phenyl]-benzamide,
2,6-dimethoxy-N-[3-((R)-1-naphthalen-1-yl-ethylcarbamoyl)-phenyl]-benzamide,
N-[3-((R)-1-naphthalen-1-yl-ethylcarbamoyl)-phenyl]-2-trifluoromethyl-benzamide,
2-methyl-N-[3-((R)-1-naphthalen-1-yl-ethylcarbamoyl)-phenyl]-benzamide,
3,4,5-trimethoxy-N-[3-((R)-1-naphthalen-1-yl-ethylcarbamoyl)-phenyl]-benzamide,
2-iodo-N-[3-((R)-1-naphthalen-1-yl-ethylcarbamoyl)-phenyl]-benzamide,
naphthalene-1-carboxylic acid [3-((R)-1-naphthalen-1-yl-ethylcarbamoyl)-phenyl]-amide,
naphthalene-2-carboxylic acid [3-((R)-1-naphthalen-1-yl-ethylcarbamoyl)-phenyl]amide,
biphenyl-4-carboxylic acid [3-((R)-1-naphthalen-1-yl-ethylcarbamoyl)-phenyl]-amide,
N-[3-((R)-1-naphthalen-1-yl-ethylcarbamoyl)-phenyl]-2-thiophen-2-yl-benzamide,
2-chloro-N-[3-((R)-1-naphthalen-1-yl-ethylcarbamoyl)-phenyl]-benzamide,
N-[3-[(naphthalen-1-ylmethyl)-carbamoyl]-phenyl-3,5-bis-trifluoromethyl-benzamide,
2-bromo-N-{3-[(naphthalen-1-ylmethyl)-carbamoyl]-phenyl}-benzamide,
2-fluoro-N-{3-[(naphthalen-1-ylmethyl)-carbamoyl]-phenyl}-benzamide,
2,6-dichloro-N-{3-((R)-1-naphthalen-1-ylmethyl)-carbamoyl]-phenyl}-benzamide,
acetic acid 2-{3-[(naphthalen-1-ylmethyl)-carbamoyl]-phenylcarbamoyl}-phenyl ester,
2-methoxy-N-[3-{(naphthalen-1-ylmethyl)-carbamoyl]phenyl}-benzamide,
2,6-dimethoxy-N-{3-[(naphthalen-1-ylmethyl)-carbamoyl]-phenyl}-benzamide,
N-{3-[(naphthalen-1-ylmethyl)-carbamoyl]-phenyl}-2-trifluoromethyl-benzamide,
N-{3-[(naphthalen-1-ylmethyl)-carbamoyl]-phenyl}-2-methyl-benzamide,
3,4,5-trimethoxy-N-{3-[(naphthalen-1-ylmethyl)-carbamoyl]-phenyl}-benzamide,
2-iodo-N-{3-[(naphthalen-1-ylmethyl)-carbamoyl]-phenyl}-benzamide,
naphthalene-1-carboxylic acid {3-[(naphthalen-1-ylmethyl)-carbamoyl]-phenyl}-amide,
naphthalene-2-carboxylic acid {3-[(naphthalen-1-ylmethyl)-carbamoyl]-phenyl}-amide,
biphenyl-4-carboxylic acid {3-[(naphthalen-1-ylmethyl)-carbamoyl]-phenyl}-amide,
N-{3-[(naphthalen-1-ylmethyl)-carbamoyl]-phenyl}-2-thiopene-2-yl-benzamide,
2-chloro-N-{3-[(naphthalen-1-ylmethyl)-carbamoyl]-phenyl}-benzamide,
naphthalene-1-carboxylic acid [3-(2-chloro-phenylcarbamoyl)-phenyl]-amide,
3-(2-bromo-benzylamino)-N-naphthalen-1-ylmethyl-benzamide,
2-trifluoromethyl-N-{3-[(2-trifluoromethyl-benzylamino)-methyl]-phenyl}-benzamide,
3-(2-trifluoromethyl-benzoylamino)-N-(2-trifluoromethyl-benzyl)-benzamide,
N-(2-bromo-benzyl)-3-(2-trifluoromethyl-benzoylamino)-benzamide,
N-(2-phenyl-benzyl)-3-(2-trifluoromethyl-benzoylamino)-benzamide,
N-(3-phenyl-benzyl)-3-(2-trifluoromethyl-benzoylamino)-benzamide,
N-(3-bromo-benzyl)-3-(2-trifluoromethyl-benzoylamino)-benzamide,
N-(2,3-dimethyl-benzyl)-3-(2-trifluoromethyl-benzoylamino)-benzamide,
N-(8-hydroxy-quinolin-2-yl)-3-(2-trifluoromethyl-benzoylamino)-benzamide,
N-(benzyl)-3-(2-trifluoromethyl-benzoylamino)-benzamide,
N-(2-methyl-benzyl)-3-(2-trifluoromethyl-benzoylamino)-benzamide,
N-(2-chloro-benzyl)-3-(2-trifluoromethyl-benzoylamino)-benzamide,
N-(2-methoxy-benzyl)-3-(2-trifluoromethyl-benzoylamino)-benzamide,
N-(2-dimethylamino-benzyl)-3-(2-trifluoromethyl-benzoylamino)-benzamide,
N-(2-piperidin-1-yl-benzyl)-3-(2-trifluoromethyl-benzoylamino)-benzamide,
N-(2-trifluoromethoxy-benzyl)-3-(2-trifluoromethyl-benzoylamino)-benzamide,
N-(2,3-dihydro-benzo[1,4]dioxin-5-ylmethyl)-3-(2-trifluoromethyl-benzoylamino)-benzamide,
N-(2-morpholin-4-yl-benzyl)-3-(2-trifluoromethyl-benzoylamino)-benzamide,
3-[2-(trifluoromethyl)-benzoylamino]-N-(3-bromo-pyridine-2-yl)-benzamide,
3-[2-(trifluoromethyl)-benzoylamino]-N-(5-methyl-pyridine-2-yl)-benzamide,
3-[2-(trifluoromethyl)-benzoylamino]-N-(5-methyl-pyridine-2-yl)-benzamide,
3-[2-(trifluoromethyl)-benzoylamino]-N-(5-cyano-pyridine-2-yl)-benzamide,
3-[2-(trifluoromethyl)-benzoylamino]-(5-trifluoromethyl-pyridine-2-yl)-benzamide,
3-[2-(trifluoromethyl)-benzoylamino]-N-(5-bromo-pyridine-2-yl)-benzamide,
3-[2-(trifluoromethyl)-benzoylamino]-(5-fluoro-pyridine-2-yl)-benzamide,
3-[2-(trifluoromethyl)-benzoylamino]-N-(5-chloro-pyridine-2-yl)-benzamide,
3-[2-(trifluoromethyl)-benzoylamino]-N-(5-iodo-pyridine-2-yl)-benzamide,
3-[2-(trifluoromethyl)-benzoylamino]-N-(5-bromo-6-methyl-pyridine-2-yl)-benzamide,
3-[2-(trifluoromethyl)-benzoylamino]-N-1,3-benzothiazol-5-yl-benzamide),
3-[2-(trifluoromethyl)-benzoylamino]-N-(2-methyl-1,3-benzothiazol-6-yl)benzamide,
3-[2-(trifluoromethyl)-benzoylamino]-N-(2-methyl-1,3-benzoxazol-6-yl)benzamide,
3-[2-(trifluoromethyl)-benzoylamino]-N-(5-chloro-1,3-benzoxazol-2-yl)benzamide,
3-[2-(trifluoromethyl)-benzoylamino]-N-(5-thien-3-ylpridin-2-yl)benzamide,
3-[2-(trifluoromethyl)-benzoylamino]-N-(5-phenylpyridin-2-yl)benzamide,
3-[2-(trifluoromethyl)-benzoylamino]-N-{5-[4-dimethylamino)phenyl]pyridin-2-yl}benzamide,
3-[2-(trifluoromethyl)-benzoylamino]-N-(2-piperidin-1-ylbenzyl)benzamide, and
3-[2-(trifluoromethyl)-benzoylamino]-N-(2-trifluoromethoxybenzyl)benzamide.

Some of the compounds of formula (I) have not hitherto been used or suggested for use in therapy. Thus, according to a further aspect, the invention provides compounds for use in therapy, selected from those compounds of formula (I) wherein

R¹ is phenyl, monosubstituted in ortho-position with R⁴;
R⁴ is selected from halogen, C1-C4-alkyl, monocyclic C5-C10-carbocyclyl, monocyclic 5 or 6-membered heterocyclyl, C1-C4-alkoxy, CF₃, NO₂, COOH, —C(O)O—C1-C4)-alkyl, and —OC(O)—C1-C4-alkyl; and
R², R³, R⁵, R⁶, X and Y are as defined herein above with respect to the compounds of formula (I); as well as pharmaceutically acceptable salts thereof.

A compound of formula (I) for use in therapy, wherein

R¹ is phenyl that is monosubstituted in ortho-position with R⁴; and
R⁴ is selected from halogen, C1-C4-alkyl, monocyclic C5-C10-carbocyclyl, monocyclic 5 or 6-membered heterocyclyl, C1-C4-alkoxy, CF₃, NO₂, COOH, —C(O)O—C1-C4)-alkyl, and —OC(O)—C1-C4-alkyl; may be represented by formula (II)

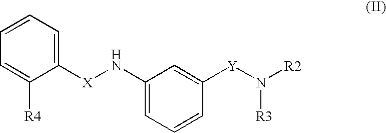

wherein R², R³, X and Y are as defined herein above with respect to the compounds of formula (I).

It should be understood that the above given description of the various embodiments of compounds according to formula (I) also applies to the compounds of formula (II) for use in therapy, provided that R¹ and R⁴ are as defined with respect to these latter compounds.

For example, in one specific embodiment, in a compound of formula (II), R² and R³ are each independently selected from H and a moiety selected from C1-C6-allyl, carbocyclyl and heterocyclyl, wherein any cyclyl is 5-7 membered monocyclic or is bicyclic having a 5- or 6-membered cycle fused with a 6-membered cycle.

In another embodiment, in a compound of formula (II), R² and R³ are each independently selected from H and a moiety selected from methyl, n-butyl, cyclohexyl, cycloheptyl, phenyl, pyridyl, quinolinyl, isoquinolinyl, benzimidazolyl, benzothiazolyl, 1H-indazolyl, indanyl and 1,2,3,4-tetrahydronaphthyl, which moiety is optionally substituted with one or more R⁵ as defined herein above, with respect to the compounds of formula (I).

Preferably, R³ is selected from H and a moiety selected from C1-C3 alkyl and monocyclic C5-C7 carbocyclyl, which moiety optionally is substituted with one or more moieties selected from monocyclic C5-C7 carbocyclyl. More preferably, R³ is H.

R⁴ preferably is selected from CH₃O, CF₃, OC(O)CH₃, NO₂, C(O)OCH₃, COOH, F, Cl, Br; I, methyl, ethyl, cyclopentyl, phenyl and thienyl.

Each R⁵ preferably is independently selected from halogen, alkyl, CN, CF₃, OH, monocyclic or bicyclic carbocyclyl and monocyclic or bicyclic heterocyclyl, wherein any cyclyl moiety may be substituted with one or more R⁶ as defined with respect to a compound of formula (I). Both X and Y preferably are CO.

In one embodiment of the invention, the compound of formula (II) for use in therapy is selected from those specific compounds enumerated herein above, which fall within the scope of formula (II) as defined herein above.

Some of the compounds of formula (I) are novel. Thus, according to a further aspect, the invention provides novel compounds, selected from those compounds of formula (I) wherein

R¹ is phenyl, monosubstituted in ortho-position with R⁴;

### R⁴ is CF₃ or Cl; and

R², R³, R⁵, R⁶, X and Y are as defined herein above with respect to the compounds of formula (I); as well as pharmaceutically acceptable salts thereof,
with the proviso that the compound is not 3-(2-chloro-benzoylamino)-N-naphtalene-1-yl-benzamide, 3-(2-chloro-benzoylamino)-N-(2,6-dimethyl-4-(thiophen-2-yl)phenyl)benzamide, or 3-(2-chloro-benzoylamino)-N-(2,6-dimethyl-4-(thiophen-2-yl)phenyl)benzamide

The first of the disclaimed compounds, i.e. 3-(2-chloro-benzoylamino)-N-naphtalene-1-yl-benzamide and 2-bromo-N-{3-[(naphthalen-1-yl)-carbamoyl]-phenyl}-benzamide, is commercially available from several sources such as ChemDiv, (http://www.chemdiv.com/) and ChemBridge (http://www.chembridge.com) etc. The latter two are disclosed for use as insecticides in WO 2006/1373767 A (cf. compounds 1-380 and I-381).

A novel compound of formula (I) wherein

R¹ is phenyl that is monosubstituted in ortho-position with R⁴; and
R⁴ is CF₃ or Cl, may be represented by formula (II)

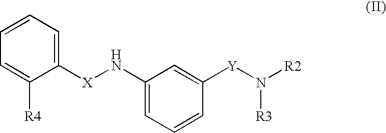

wherein R², R³, X and Y are as defined herein above with respect to the compounds of formula (I).

Preferably, R⁴ in formula (II) is CF₃.

It should be understood that the above-given description of the various embodiments of compounds according to formula (I) also applies to the novel compounds of formula (II) provided that R¹ and R⁴ are as defined with respect to the novel compounds of formula (II) according to the invention.

For example, in one preferable embodiment of the invention, in a novel compound of formula (II):

### R⁴ is CF₃;

X and Y are both CO;

### R³ is H; and

R² is as defined herein above with respect to the compounds of formula (I).

In another preferable embodiment of the invention, in a novel compound of formula (II):

### R⁴ is CF₃;

R² is pyridin-2-yl, substituted with 1 or 2 R⁵, R⁵ being as defined herein above with respect to the compounds of formula (I); and
X, Y and R³ are as defined herein above with respect to the compounds of formula (I), preferably X and Y are CO and R³ is H.

In still another preferable embodiment of the invention, in a novel compound of formula (II):

### R⁴ is CF₃;

R² is pyridin-2-yl, substituted with 2 R⁵, one of which is in para-position (i.e. in position 5 on the pyridine ring), R⁵ being as defined herein above with respect to the compounds of formula (I); and
X, Y and R³ are as defined herein above with respect to the compounds of formula (I), preferably X and Y are CO and R³ is H.

In another preferable embodiment of the invention, in a novel compound of formula (II):

### R⁴ is CF₃;

R² is pyridin-2-yl, monosubstituted with R⁵, R⁵ being as defined herein above with respect to the compounds of formula (I);
X, Y and R³ are as defined herein above with respect to the compounds of formula (I), preferably X and Y are CO and R³ is H.

In one further preferred embodiment, R⁴ is CF₃;

R² is pyridin-2-yl, monosubstituted in para-position (i.e. at position 5 on the pyridine ring). with R⁵, R⁵ being as defined herein above with respect to the compounds of formula (I), and
X, Y and R³ are as defined herein above with respect to the compounds of formula (I), preferably X and Y are CO and R³ is H.

In a particularly preferable embodiment of the invention, in a novel compound of formula (II):

### R⁴ is CF₃;

R² is pyridin-2-yl, monosubstituted in para-position (i.e. at position 5 on the pyridine ring). with R⁵, as defined herein above;
R⁵ is selected from CF₃, halogen, C1-C3 alkyl, and CN; and
X, Y and R³ are as defined herein above with respect to the compounds of formula (I);
preferably X and Y are CO and R³ is H.

In another preferable embodiment of the invention, in a novel compound of formula (II), R² is bicyclic heterocyclyl, preferably a 5- or 6-membered heterocycle containing 1 or 2 heteroatoms in the ring, fused with a 6-membered carbocycle, such as a 5- or 6-membered heterocycle fused with phenyl, e.g. quinolyl, isoquinolyl, 1H-indazolyl, 1H-benzimidazolyl, 1,3-benzothiazolyl, 1,3-benzoxazolyl.

In one embodiment of the invention, the novel compound of formula (II) for use in therapy is selected from those specific compounds enumerated herein above, which fall within the scope of formula (II), as defined with respect to the novel compounds of the invention.

In particular, the novel compound of formula (II) may suitably be selected from
3-[2-(Trifluoromethyl)-benzoylamino]-N-(3-bromo-pyridine-2-yl)-benzamide,
3-[2-(Trifluoromethyl)-benzoylamino]-N-(5-methyl-pyridine-2-yl)-benzamide,
3-[2-(Trifluoromethyl)-benzoylamino]-N-(5-trifluoromethyl-pyridine-2-yl)-benzamide,
3-[2-(Trifluoromethyl)-benzoylamino]-N-(5-cyano-pyridine-2-yl)-benzamide,
3-[2-(Trifluoromethyl)-benzoylamino]-N-(5-bromo-pyridine-2-yl)-benzamide,
3-[2-(Trifluoromethyl)-benzoylamino]-N-(5-fluoro-pyridine-2-yl)-benzamide,
3-[2-(Trifluoromethyl)-benzoylamino]-N-(5-chloro-pyridine-2-yl)-benzamide,
3-[2-(Trifluoromethyl)-benzoylamino]-N-(5-iodo-pyridine-2-yl)-benzamide,
3-[2-(Trifluoromethyl)-benzoylamino]-N-(5-bromo-6-methyl-pyridine-2-yl)-benzamide, and
3-[2-(trifluoromethyl)-benzoylamino]-N-1,3-benzothiazol-5-yl-benzamide.

### Methods of Synthesis

The compounds of formula (I) (including those of formula (II)) of the invention can be prepared according to the synthetic routes outlined herein and by following the methods described herein below and illustrated in the Examples.

### Example 1

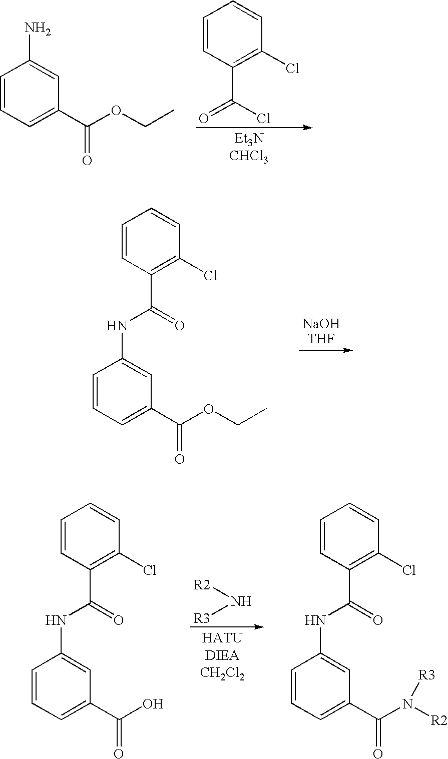

### 3-(2-Chloro-benzoylamino)-benzoic acid ethyl ester

Ethyl-3-aminobenzoate (5.95 g, 36.0 mmol) was dissolved in CHCl₃ (100 mL) and Et₃N (15.1 mL, 108 mmol) was added. To the stirred solution 2-Chloro-benzoylchloride (6.85 mL, 54.0 mmol) was added dropwise. The solution was stirred overnight and then extracted with brine. The organic phase was dried and evaporated. The crude product was purified on silica gel with isohexane:EtOAc (85:15) to give the pure product in quantitative yield.

NMR ¹H(CDCl₃): δ 8.2-7.2 (m, 8H), 4.4 (q, 2H), 1.4 (t, 3H); MS M/z: 303.8.

### 3-(2-Chloro-benzoylamino)-benzoic acid

3-(2-Chloro-benzoylamino)-benzoic acid ethyl ester (1.00 g, 3.29 mmol) was dissolved in THF (100 mL). NaOH (aq.) (100 mL, 1M) was added to the solution. The reaction mixture was refluxed overnight. The THF was evaporated and the aqueous solution was acidified with conc. HCl. The product started to precipitate. The acidification was continued until pH approx. 4. The product was filtered off and dried under vacuum.

MS M/z: 275.8

### General Procedure for Compounds 2-6:

3-(2-Chloro-benzoylamino)-benzoic acid (50 mg, 0.18 mmol) was dissolved in CH₂Cl₂ (2 mL), HATU (82.8 mg, 0.218 mmol) and amine (1.2 equiv., 0.218 mmol) was added. To the stirred solution was added N,N-diisopropylethylamine (60.5 μL, 0.363 mmol). The reaction mixture was stirred overnight and thereafter evaporated. The crude product was purified on circular chromatography.

### 3-(2-Chloro-benzoylamino)-N-benzyl-benzamide (2)

Prepared according to the general procedure using benzylamine (23.8 μL, 0.218 mmol). The product was purified on circular chromatography (Hexan:EtOAc, 10-40%) to obtain the pure title compound in 53% yield.

NMR ¹H (CDCl₃): δ 8.79 (s, 1H), 8.07 (s, 1H), 7.95 (d, 1H), 7.57 (d, 1H), 7.46 (d, 1H), 7.2-7.4 (m, 7H), 6.72 (t, 1H), 4.38 (d, 2H); MS M/z: 365.0

### 3-(2-Chloro-benzoylamino)-N-(2-methyl-quinolin-4-yl)-benzamide (3)

Prepared according to the general procedure using 4-aminoquinaldine (35.1 μL, 0.218 mmol). The product was purified on circular chromatography (CH₂Cl₂:MeOH, 5%) to obtain the pure title compound in 24% yield. MS M/z: 415.9

### 3-(2-Chloro-benzoylamino)-N-butyl-benzamide (4)

Prepared according to the general procedure using butylamine (21.5 μL, 0.218 mmol). The product was purified on circular chromatography (Hexane:EtOAc, 10-40%) to obtain the pure title compound in 67% yield.

NMR ¹H (CDCl₃): δ 8.12 (s, 1H), 8.04 (bs, 1H), 7.80 (dd, 2H), 7.58 (d, 1H), 7.38-7.48 (m, 4H), 6.18 (bs, 1H), 3.43 (q, 2H), 1.81 (m, 2H), 1.42 (m, 2H), 0.98 (t, 3H); MS M/z: 330.9

### 3-(2-Chloro-benzoylamino)-N,N-dibenzyl-benzamide (5)

Prepared according to the general procedure using dibenzylamine (41.8 μL, 0.218 mmol). The product was purified on circular chromatography (Hexane:EtOAc, 10-40%) to obtain the pure title compound in 96% yield. MS M/z: 454.9

### 3-(2-Chloro-benzoylamino)-N-(diphenylyl-methyl)-benzamide (6)

Prepared according to the general procedure using diphenylmethylamine (37.5 μL, 0.218 mmol). The product was purified on circular chromatography (Hexane:EtOAc, 10-40%) to obtain the pure title compound in 17% yield. MS M/z: 440.9

### General Procedure for Compounds 7-11 and 13-15:

3-(2-Chloro-benzoylamino)-benzoic acid (40 mg, 0.15 mmol) was dissolved in CH₂Cl₂ (2 mL), HATU (66.2 mg, 0.174 mmol) and amine (1.2 equiv., 0.174 mmol) was added. To the stirred solution was added N,N-diisopropylethylamine (48.4 μL, 0.290 mmol). The reaction mixture was stirred overnight and thereafter evaporated. The crude product was purified on preparative LC/MS (water:acetonitrile 30-80%).

### 3-(2-Chloro-benzoylamino)-N-(isoquinolin-5-yl-)-benzamide (7)

Prepared according to the general procedure using 5-aminoisoquinoline (25.1 mg, 0.174 mmol). The product was purified on preparative LC/MS (water:acetonitrile 30-80%) to obtain the pure title compound in 34% yield. MS M/z: 401.8.

### 3-(2-Chloro-benzoylamino)-N-(quinolin-8-yl-)-benzamide (8)

Prepared according to the general procedure using 8-aminoquinoline (25.1 mg, 0.174 mmol). The product was purified on preparative LC/MS (water:acetonitrile 30-80%) to obtain the pure title compound in 22% yield.

NMR ¹H (CDCl₃): δ 10.73 (s, 1H), 8.84 (m, 2H), 8.35 (s, 1H), 8.19 (m, 3H), 7.82 (d, J=7.3 Hz, 1H), 7.73 (m, 1H), 7.56 (m, 3H), 7.48 (dd, J=8.1, 3.4 Hz, 1H), 7.39 (m, HT), 7.32 (m, 2H); MS M/z: 401.9.

### 3-(2-Chloro-benzoylamino)-N-(quinolin-2-yl-)-benzamide (9)

Prepared according to the general procedure using 2-aminoquinoline (25.1 mg, 0.174 mmol). The product was purified on preparative LC/MS (water:acetonitrile 30-80%) to obtain the pure title compound in 37% yield. MS M/z: 401.9.

### 3-(2-Chloro-benzoylamino)-N-(pyridine-2-ylmethyl)-benzamide (10)

Prepared according to the general procedure using 2-picolylamine (17.8 μL, 0.174 mmol). The product was purified on preparative LC/MS (water:acetonitrile 30-80%) to obtain the pure title compound in 68% yield. MS M/z: 365.9.

### 3-(2-Chloro-benzoylamino)-N-(pyridine-3-ylmethyl)-benzamide (11)

Prepared according to the general procedure using 3-picolylamine (17.6 μL, 0.174 mmol). The product was purified on preparative LC/MS (water:acetonitrile 30-80%) to obtain the pure title compound in 15% yield.

NMR ¹H (CDCl₃): δ 8.61 (s, 1H), 8.51 (d, J=4.8 Hz, 1H), 8.25 (s, 1H), 8.13 (s, 1H), 7.82 (d, J=8.2 Hz, 1H), 7.73 (t, J=7.4 Hz, 2H), 7.60 (d, J=7.4 Hz, 1H), 7.43 (t, J=7.9 Hz, 2H), 7.39 (m, 2H), 7.30 (m, 1H), 6.91 (m, 1H), 4.65 (d, J=6.4 Hz, 2H); MS M/z: 365.9.

### 3-(2-Chloro-benzoylamino)-N-(1H-benzimidazol-2-yl)-benzamide (13)

Prepared according to the general procedure using 2-aminobenzimidazole (23.2 mg, 0.174 mmol). The product was purified on preparative LC/MS (water:acetonitrile 30-80%) to obtain the pure title compound in 10% yield. MS M/z: 390.9

### 3-(2-Chloro-benzoylamino)-N-(benzothiazol-6-yl)-benzamide (14)

Prepared according to the general procedure using 6-aminobenzothiazole (26.2 mg, 0.174 mmol). The product was purified on preparative LC/MS (water:acetonitrile 30-80%) to obtain the pure title compound in 74% yield. MS M/z: 407.8

### 3-(2-Chloro-benzoylamino)-N-(1H-indazol-5-yl)-benzamide (15)

Prepared according to the general procedure using 5-aminoindazole (23.2 mg, 0.174 mmol). The product was purified on preparative LC/MS (water:acetonitrile 30-80%) to obtain the pure title compound in 62% yield. MS M/z: 390.9

### General Procedure for Compounds 12 and 33-36:

3-(2-Chloro-benzoylamino)-benzoic acid (30 mg, 0.11 mmol) was dissolved in CH₂Cl₂ (2 mL), HATU (49.7 mg, 0.131 mmol) and amine (1.2 equiv., 0.131 mmol) was added. To the stirred solution was added N,N-diisopropylethylamine (36.3 μL, 0.218 mmol). The reaction mixture was stirred overnight and thereafter evaporated. The crude product was purified on preparative LC/MS (water:acetonitrile 30-80%).

### 3-(2-Chloro-benzoylamino)-N-(pyridine-2-yl)-benzamide (12)

Prepared according to the general procedure using 2-aminopyridine (12.3 mg, 0.131 mmol). The product was purified on preparative LC/MS (water:acetonitrile 30-80%) to obtain the pure title compound in 21% yield. MS M/z: 351.9.

### 3-(2-Chloro-benzoylamino)-N,N-dicyclohexyl-benzamide (33)

Prepared according to the general procedure using dicyclohexyl amine (26.0 μL, 0.131 mmol). The product was purified on preparative LC/MS (water:acetonitrile 30-80%) to obtain the pure title compound in 21% yield. MS M/z: 439.1.

### 3-(2-Chloro-benzoylamino)-N-indan-1-yl-benzamide (34)

Prepared according to the general procedure using indan-1-ylamine (16.8 μL. 0.131 mmol). The product was purified on preparative LC/MS (water:acetonitrile 30-80%) to obtain the pure title compound in 53% yield. MS M/z: 391.0.

### 3-(2-Chloro-benzoylamino)-N-(1,2,3,4-tetrahydro-naphthalen-1-yl)-benzamide (35)

Prepared according to the general procedure using 1,2,3,4-tetrahydro-1-naphthylamine (18.7 μL, 0.131 mmol). The product was purified on preparative LC/MS (water:acetonitrile 30-80%) to obtain the pure title compound in 37% yield. MS M/z: 405.0.

### 3-(2-Chloro-benzoylamino)-N-cycloheptyl-benzamide (36)

Prepared according to the general procedure using cycloheptylamine (16.6 μL, 0.131 mmol). The product was purified on preparative LC/MS (water:acetonitrile 30-80%) to obtain the pure title compound in 30% yield.

NMR ¹H (CDCl₃): δ 8.03 (s, 1H), 8.01 (s, 1H), 7.82 (d, J=7.3 Hz, 1H), 7.79 (dd, J=7.3, 1.5 Hz, 1H), 7.55 (d, J=7.9 Hz, 1H), 7.43 (m, 4H), 6.11 (d, J=7.5 Hz, 1H), 4.17 (m, 1H), 2.05 (m, 2H), 1.69 (m, 4H), 1.58 (m, 6H); MS M/z: 371.0.

### Example 2

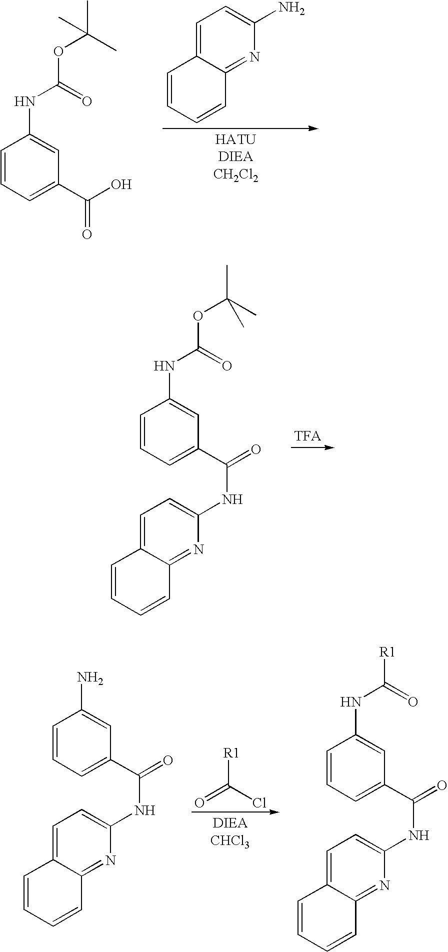

### [3-(Quinolin-2-ylcarbamoyl)-phenyl]-carbamic acid tert-butyl ester (16)

3-(Boc-amino)benzoic acid (1.00 g, 3.63 mmol) was dissolved in CH₂Cl₂ (10 mL), HATU (1.66 g, 4.35 mmol) and 2-aminoquinoline (628 mg, 4.35 mmol) was added. To the stirred solution was added N,N-diisopropylethylamine (1.21 mL, 7.25 mmol). The reaction mixture was stirred overnight and thereafter washed with water and dried and evaporated. The crude product was purified on column chromatography (isohexane:EtOAc 9:1) to obtain the title compound in 79% yield. MS M/z: 364.0.

### 3-Amino-N-quinolin-2-yl-benzamide

[3-(Quinolin-2-ylcarbamoyl)-phenyl]carbamic acid tert-butyl ester (1.04 g, 2.86 mmol) was dissolved in TFA (100 mL) and stirred for 45 min. The reaction mixture was evaporated. The crude product was used without further purification. MS M/z: 263.9

### General Procedure for Compounds 17-32 and 37-53:

3-Amino-N-quinolin-2-yl-benzamide (30 mg, 0.11 mmol) was dissolved in CHCl₃ (2 mL) and N,N-diisopropylethylamine (188 μL, 1.14 mmol) was added. To the stirred solution acid chloride (1.5 equiv., 0.171 mmol) was added and the reaction mixture was stirred overnight. The reaction mixture was evaporated and the crude product was purified on preparative LC/MS (water:acetonitrile 30-80%).

### 3-Benzoylamino-N-quinolin-2-yl-benzamide (17)

Prepared according to the general procedure using benzoyl chloride (19.8 μL, 0.171 mmol). The product was purified on preparative LC/MS (water:acetonitrile 30-80%) to obtain the pure title compound in 28% yield. MS M/z: 367.9.

### 3-Acetylamino-N-quinolin-2-yl-benzamide (18)

Prepared according to the general procedure using acetyl chloride (12.2 pt, 0.171 mmol). The product was purified on preparative LC/MS (water:acetonitrile 30-80%) to obtain the pure title compound in 23% yield. MS M/z: 305.9.

### Thiophene-2-carboxylic acid [3-(quinolin-2-ylcarbamoyl)-phenyl]-amide (19)

Prepared according to the general procedure using 2-thionyl carbonyl chloride (18.3 mL, 0.171 mmol). The product was purified on preparative LC/MS (water:acetonitrile 30-80%) to obtain the pure title compound in 28% yield.

NMR ¹H (CDCl₃): δ 8.95 (bs, 1H), 8.52 (d, J=8.9 Hz, 1H), 8.20 (d, J=9.1 Hz, 1H), 8.1.2 (m, 1H), 8.05 (m, 2H), 7.83 (d, J=8.5 Hz, 1H), 7.79 (d, J=8.5 Hz, 1H), 7.73 (d, J=7.6 Hz, 1H), 7.68 (m, 2H), 7.56 (dd, J=5.1, 1.2 Hz, 1H), 7.48 (m, 1H), 7.12 (dd, J=5.2, 3.9 Hz, 1H); MS M/z: 373.9.

### Furan-2-carboxylic acid [3-(quinolin-2-ylcarbamoyl)-phenyl]-amide (20)

Prepared according to the general procedure using 2-furyl carbonyl chloride (16.9 μL, 0.171 mmol). The product was purified on preparative LC/MS (water:acetonitrile 30-80%) to obtain the pure title compound in 21% yield. MS M/z: 357.9.

### N-[3-(Quinolin-2-ylcarbamoyl)-phenyl]-nicotinamide (21)

Prepared according to the general procedure using nicotinyl chloride hydrochloride (30.4 mg, 0.171 mmol). The product was purified on preparative LC/MS (water:acetonitrile 30-80%) to obtain the pure title compound in 12% yield. MS M/z: 368.9.

### N-[3-(Quinolin-2-ylcarbamoyl)-phenyl]-isonicotinamide (22)

Prepared according to the general procedure using isonicotinyl chloride hydrochloride (30.4 mg, 0.171 mmol). The product was purified on preparative LC/MS (water:acetonitrile 30-80%) to obtain the pure title compound in 22% yield. MS M/z: 369.0.

### 3-Phenylacetylamino-N-quinolin-2-yl-benzamide (23)

Prepared according to the general procedure using phenylacetyl chloride (22.6 μL, 0.171 mmol). The product was purified on preparative LC/MS (water:acetonitrile 30-80%) to obtain the pure title compound in 24% yield. MS M/z: 382.0.

### 3-(Cyclopropanecarbonyl-amino)-N-quinolin-2-yl-benzamide (24)

Prepared according to the general procedure using cyclopropylcarboxy chloride (15.4 μL, 0.171 mmol). The product was purified on preparative LC/MS (water:acetonitrile 30-80%) to obtain the pure title compound in 27% yield. MS M/z: 331.9.

### 3-Diphenylacetylamino-N-quinolin-2-yl-benzamide (25)

Prepared according to the general procedure using diphenyl acetyl chloride (39.4 mg, 0.171 mmol). The product was purified on preparative LC/MS (water:acetonitrile 30-80%) to obtain the pure title compound in 15% yield. MS M/z: 458.0.

### 3-(3-Cyclopentyl-propionylamino)-N-quinolin-2-yl-benzamide (26)

Prepared according to the general procedure using 3-cyclopentylpropionyl chloride (26.4 μL, 0.171 mmol). The product was purified on preparative LC/MS (water:acetonitrile 30-80%) to obtain the pure title compound in 22% yield. MS M/z: 388.0.

### 3-[(E)-(3-Phenyl-acryloyl)amino]-N-quinolin-2-yl-benzamide (27)

Prepared according to the general procedure using (E)-3-phenyl-acryloyl chloride (28.5 mg, 0.171 mmol). The product was purified on preparative LC/MS (water:acetonitrile 30-80%) to obtain the pure title compound in 15% yield. MS M/z: 394.0.

### 3,4-Dichloro-N-[3-(quinolin-2-ylcarbamoyl)-phenyl]-benzamide (28)

Prepared according to the general procedure using 3,4-dichlorobenzoyl chloride (35.8 mg, 0.171 mmol). The product was purified on preparative LC/MS (water:acetonitrile 30-80%) to obtain the pure title compound in 8% yield. MS M/z: 435.8.

### 3-(3-Chloro-benzoylamino)-N-quinolin-2-yl-benzamide (29)

Prepared according to the general procedure using 3-chlorobenzoyl chloride (21.9 μL, 0.171 mmol). The product was purified on preparative LC/MS (water:acetonitrile 30-80%) to obtain the pure title compound in 22% yield.

NMR ¹H (CDCl₃): δ 8.40 (d, J=9.2 Hz, 1H), 8.16 (d, J=9.2 Hz, 1H), 8.05 (m, 1H), 7.93 (m, 1H), 7.86 (t, J=1.7 Hz, 1H), 7.74 (m, 4H), 7.68 (m, 1H), 7.59 (m, 1H), 7.48-7.32 (m, 5H); MS M/z: 401.9.

### 3-(4-Methoxy-benzoylamino)-N-quinolin-2-yl-benzamide (30)

Prepared according to the general procedure using p-anisolyl chloride (29.2 mg, 0.171 mmol). The product was purified on preparative LC/MS (water:acetonitrile 30-80%) to obtain the pure title compound in 14% yield. MS M/z: 397.9.

### 3-(4-Chloro-benzoylamino)-N-quinolin-2-yl-benzamide (31)

Prepared according to the general procedure using 4-chlorobenzoyl chloride (21.8 μL, 0.171 mmol). The product was purified on preparative LC/MS (water:acetonitrile 30-80%) to obtain the pure title compound in 9% yield. MS M/z: 401.9.

### 3-(3-Trifluoromethyl-benzoylamino)-N-quinolin-2-yl-benzamide (32)

Prepared according to the general procedure using 4-Trifluoromethylbenzoyl chloride (35.6 mg, 0.171 mmol). The product was purified on preparative LC/MS (water:acetonitrile 30-80%) to obtain the pure title compound in 5% yield. MS M/z: 435.9.

Similarly, the following compounds were prepared according to the general procedure using the appropriate reagents:

### 3-(3,5-Bis-trifluoromethyl-benzoylamino)-N-quinolin-2-yl-benzamide (37)

MS M/z: 504.2.

### 3-(2-Bromo-benzoylamino)-N-quinolin-2-yl-benzamide (38)

MS M/z: 446.1.

### 3-(2-Fluoro-benzoylamino)-N-quinolin-2-yl-benzamide (39)

MS M/z: 386.2.

### 3-(2,6-Chloro-benzoylamino)-N-quinolin-2-yl-benzamide (40)

MS M/z: 438.1.

### 3-(2-Methoxy-benzoylamino)-N-quinolin-2-yl-benzamide (41)

MS M/z: 398.2.

### 3-(2,6-Dimethoxy-benzoylamino)-N-quinolin-2-yl-benzamide (42)

MS M/z: 428.2.

### 3-(2-Trifluoromethyl-benzoylamino)-N-quinolin-2-yl-benzamide (43)

MS M/z: 436.2.

### 3-(2-Methyl-benzoylamino)-N-quinolin-2-yl-benzamide (44)

MS M/z: 382.2.

### 3-(3,4,5-Trimethoxy-benzoylamino)-N-quinolin-2-yl-benzamide (45)

MS M/z: 458.2.

### 3-(2-Iodo-benzoylamino)-N-quinolin-2-yl-benzamide (46)

MS M/z: 494.1.

### Naphthalene-1-carboxylic acid [3-(quinolin-2-ylcarbamoyl)-phenyl]amide (47)

MS M/z: 418.2.

### Naphthalene-2-carboxylic acid [3-(quinolin-2-ylcarbamoyl)-phenyl]-amide (48)

MS M/z: 418.2.

### 3-(2-Thiophene-2-yl-benzoylamino)-N-quinolin-2-yl-benzamide (49)

MS M/z: 450.2.

### Acetic acid 2-[3-(quinolin-2-ylcarbamoyl)-phenylcarbamoyl]-phenyl ester (50)

MS M/z: 426.2.

### Biphenyl-4-carboxylic acid [3-(quinolin-2-ylcarbamoyl)-phenyl]-amide (51)

MS M/z: 444.2.

### 3-(2-Ethyl-benzoylamino)-N-quinolin-2-yl-benzamide (52)

MS M/z: 396.2.

### 3-(2-Nitro-benzoylamino)-N-quinolin-2-yl-benzamide (53)

MS M/z: 413.2.

### Example 3

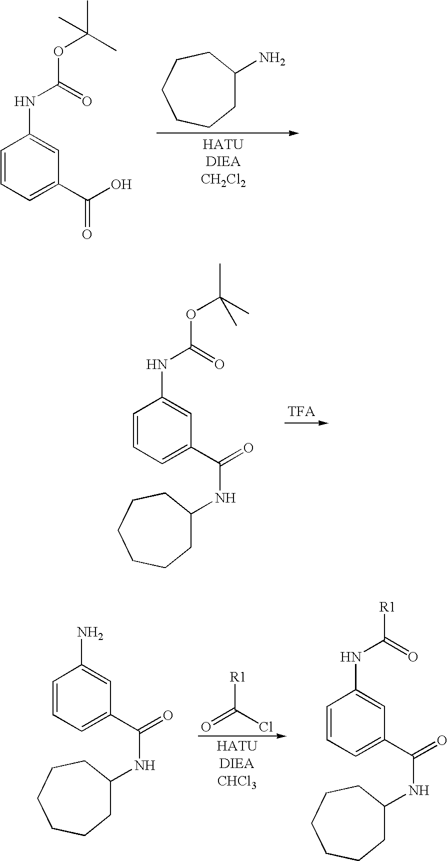

### (3-Cycloheptylcarbamoyl-phenyl)-carbamic acid tert-butyl ester

3-(Boc-amino)benzoic acid (1.00 g, 4.21 mmol) was dissolved in CH₂Cl₂ (10 mL), HATU (1.92 g, 5.06 mmol) and cycloheptylamine (644 μL, 5.06 mmol) was added. To the stirred solution was added N,N-diisopropylethylamine (1.41 mL, 8.43 mmol). The reaction mixture was stirred overnight and thereafter washed with water and dried and evaporated. The crude product was purified on column chromatography (isohexane:EtOAc 5:1) to obtain the title compound in 71% yield. MS M/z: 333.1.

### 3-Amino-N-cycloheptyl-benzamide

[3-(Cycloheptylcarbamoyl)-phenyl]-carbamic acid tert-butyl ester (1.00 g, 3.01 mmol) was dissolved in TFA (100 mL) and stirred for 45 min. The reaction mixture was evaporated. The crude product was used without further purification. MS M/z: 233.1

### General Procedure for Compounds 54-69:

3-Amino-N-cycloheptyl-benzamide (15 mg, 0.060 mmol) was dissolved in CH₂Cl₂ (2 mL), HATU (29.5 mg, 0.0775 mmol) and carboxylic acid (1.2 equiv., 0.0775 mmol) was added. To the stirred solution was added N,N-diisopropylethylamine (21.5 μL, 0.129 mmol). The reaction mixture was stirred overnight and thereafter evaporated. The crude product was purified on preparative LC/MS (water:acetonitrile 30-80%).

### N-(3-Cycloheptylcarbamoyl-phenyl)-2-fluorobenzamide (63)

Prepared according to the general procedure using 2-fluorobenzoic acid (10.9 mg, 0.0775 mmol). The product was purified on preparative LC/MS (water:acetonitrile 30-80%) to obtain the pure title compound in 32% yield. MS M/z: 355.1.

### N-(3-Cycloheptylcarbamoyl-phenyl)-2-methoxy-benzamide (64)

Prepared according to the general procedure using 2-methoxybenzoic acid (11.8 mg, 0.0775 mmol). The product was purified on preparative LC/MS (water:acetonitrile 30-80%) to obtain the pure title compound in 36% yield.

NMR ¹H (CDCl₃): δ 9.92 (s, 1H), 8.27 (dd, J=7.9, 1.7 Hz, 1H), 8.03 (s, 1H), 7.91 (m, 1H), 7.51 (m, 2H), 7.42 (t, J=7.7 Hz, 1H), 7.15 (t, J=7.7 Hz, 1H), 7.03 (t, J=7.7 Hz, 1H), 6.18 (d, J=7.5 Hz, 1H), 4.18 (m, 1H), 4.08 (s, 3H), 2.03 (m, 2H), 2.63 (m, 4H), 2.59 (m, 6H); MS M/z: 367.1.

### N-(3-Cycloheptylcarbamoyl-phenyl)-2,6-dimethoxy-benzamide (65)

Prepared according to the general procedure using 2,6-dimethoxybenzoic acid (14.1 mg, 0.0775 mmol). The product was purified on preparative LC/MS (water:acetonitrile 30-80%) to obtain the pure title compound in 6% yield. MS M/z: 397.1.

### Acetic acid 2-(3-cycloheptylcarbamoyl-phenylcarbamoyl)-phenyl ester (66)

Prepared according to the general procedure using acetylsalicylic acid (14.0 mg, 0.0775 mmol). The product was purified on preparative LC/MS (water:acetonitrile 30-80%) to obtain the pure title compound in 24% yield. MS M/z: 395.1.

### N-(3-Cycloheptylcarbamoyl-phenyl)-phthalamic acid methyl ester (67)

Prepared according to the general procedure using monomethoxyphtalic acid (14.0 mg, 0.0775 mmol). The product was purified on preparative LC/MS (water:acetonitrile 30-80%) to obtain the pure title compound in 27% yield. MS m/z: 395.1.

### 2-Bromo-N-(3-cycloheptylcarbamoyl-phenyl)-benzamide (68)

Prepared according to the general procedure using 2-bromobenzoic acid (15.6 mg, 0.0775 mmol). The product was purified on preparative LC/MS (water:acetonitrile 30-80%) to obtain the pure title compound in 27% yield. MS M/z: 417.0.

### N-(3-Cycloheptylcarbamoyl-phenyl)-2-iodo-benzamide (69)

Prepared according to the general procedure using 2-iodobenzoic acid (19.2 mg, 0.0775 mmol). The product was purified on preparative LC/MS (water:acetonitrile 30-80%) to obtain the pure title compound in 20% yield.

NMR ¹H (CDCl₃): δ 7.99 (s, 1H), 7.92 (d, J=7.7 Hz, 1H), 7.83 (d, J=7.7 Hz, 1H), 7.61 (s, 1H), 7.54 (m, 2H), 7.43 (m, 2H), 7.18 (m, 1H), 6.10 (d, J=7.5 Hz, 1H), 4.13 (m, 1H), 2.04 (m, 2H), 2.69 (m, 4H), 2.59 (m, 6H); MS M/z: 463.0.

Similarly, the following compounds were prepared according to the general procedure using the appropriate reagents:

### N-(3-Cycloheptylcarbamoyl-phenyl)-3,5-bis-trifluoromethyl-benzamide (54)

MS M/z: 473.2.

### 2,6-Dichloro-N-(3-cycloheptylcarbamoyl-phenyl)-benzamide (55)

MS M/z: 407.2.

### N-(3-Cycloheptylcarbamoyl-phenyl)-2-trifluoromethylbenzamide (56)

MS M/z: 405.2.

### N-(3-Cycloheptylcarbamoyl-phenyl)-2-methylbenzamide (57)

MS M/z: 351.3.

### N-(3-Cycloheptylcarbamoyl-phenyl)-3,4,5-trimethoxy-benzamide (58)

MS M/z: 427.3.

### Naphthalene-1-carboxylic acid (3-cycloheptylcarbamoyl-phenyl)-amide (59)

MS M/z: 387.3.

### Naphthalene-2-carboxylic acid (3-cycloheptylcarbamoyl-phenyl)-amide (60)

MS M/z: 387.3.

### Biphenyl-4-carboxylic acid (3-cycloheptylcarbamoyl-phenyl)-amide (61)

MS M/z: 413.3.

### N-(3-Cycloheptylcarbamoyl-phenyl)-2-thiophene-2-yl-benzamide (62)

MS M/z: 419.2.

### N-(3-Cycloheptylcarbamoyl-phenyl)-phthalamic acid (70)

3-Amino-N-cycloheptyl-benzamide (15 mg, 0.060 mmol) was dissolved in CH₂Cl₂ (2 mL) and phthalic anhydride (11.5 mg, 0.0775 mmol) was added. To the stirred solution was added N,N-diisopropylethylamine (21.5 μL, 0.129 mmol). The reaction mixture was stirred overnight and thereafter evaporated. The crude product was purified on preparative LC/MS (water:acetonitrile 30-80%) to obtain the title compound in 43% yield. MS M/z: 381.1.

### Example 4

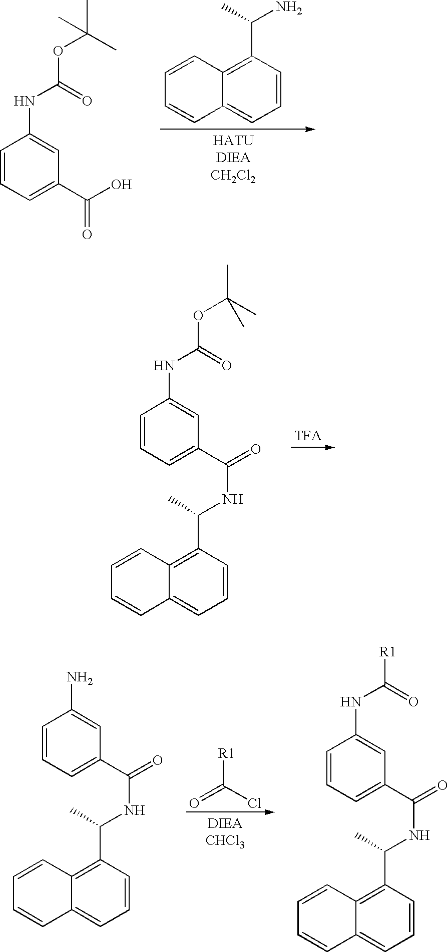

### [3-((S)-1-Naphthalen-1-yl-ethylcarbamoyl)-phenyl]-carbamic acid tert-butyl ester

3-(Boc-amino)benzoic acid (500 mg, 2.11 mmol) was dissolved in CH₂Cl₂ (5 mL), HATU (961 mg, 2.53 mmol) and (S)-1-naphthalen-1-yl-ethylamine (406 μL, 2.53 mmol) was added. To the stirred solution was added N,N-diisopropylethylamine (703 μL, 4.21 mmol). The reaction mixture was stirred overnight. The product precipitated from the reaction mixture. The product was filtered off and washed with CH₂Cl₂ and dried to obtain the title compound in 62% yield. MS M/z: 391.1.

### 3-Amino-N—((S)-1-naphthalen-1-yl-ethyl)-benzamide

[3-((S)-1-Naphthalen-1-yl-ethylcarbamoyl)-phenyl]carbamic acid tert-butyl ester (513 mg, 1.31 mmol) was dissolved in TFA (50 mL) and stirred for 45 min. The reaction mixture was evaporated. The crude product was used without further purification. MS M/z: 291.1.

### General Procedure for Compounds 71-85:

3-Amino-N—((S)-1-naphthalen-1-yl-ethyl)-benzamide (30 mg, 0.10 mmol) was dissolved in CHCl₃ (2 mL) and N,N-diisopropylethylamine (171 μL, 1.03 mmol) was added. To the stirred solution acid chloride (1.5 equiv., 0.155 mmol) was added and the reaction mixture was stirred overnight. The reaction mixture was evaporated and the crude product was purified on preparative LC/MS (water:acetonitrile 30-80%).

### 2-Chloro-N-[3-((S)-1-naphthalen-1-yl-ethylcarbamoyl)-phenyl]-benzamide (85)

Prepared according to the general procedure using 2-chlorobenzoic acid chloride (27.1 mg, 0.155 mmol). The product was purified on preparative LC/MS (water:acetonitrile 30-80%) to obtain the pure title compound in 35% yield.

NMR ¹H (CDCl₃): δ 8.23 (s, 1H), 8.11 (d, J=8.5 Hz, 1H), 7.99 (s, 1H), 7.85 (m, 2H), 7.80 (d, J=8.2 Hz, 1H), 7.67 (dd, J=7.1, 1.0 Hz, 1H), 7.58 (d, J=7.6 Hz, 1H), 7.44 (m, 4H), 7.37 (m, 4H), 6.48 (d, J=7.9 Hz, 1H), 6.02 (m, 1H), 1.77 (d, J=6.7 Hz, 3H); MS M/z: 429.0

Similarly, the following compounds were prepared according to the general procedure using the appropriate reagents:

### N-[3-((S)-1-Naphthalen-1-yl-ethylcarbamoyl)-phenyl]-3,5-bis-trifluoromethyl-benzamide (71)

MS M/z: 531.2.

### 2-Bromo-N-[3-((S)-1-naphthalen-1-yl-ethylcarbamoyl)-phenyl]-benzamide (72)

MS M/z: 473.2.

### 2-Fluoro-N-[3-((S)-1-naphthalen-1-yl-ethylcarbamoyl)-phenyl]-benzamide (73)

MS M/z: 413.2.

### 2,6-Dichloro-N-[3-((S)-1-naphthalen-1-yl-ethylcarbamoyl)-phenyl]-benzamide (74)

MS M/z: 465.1.

### 2-Methoxy-N-[3-((S)-1-naphthalen-1-yl-ethylcarbamoyl)-phenyl]-benzamide (75)

MS M/z: 425.2.

### 2,6-Dimethoxy-N-[3-((S)-1-naphthalen-1-yl-ethylcarbamoyl)-phenyl]-benzamide (76)

MS M/z: 455.3.

### N-[3-((S)-1-Naphthalen-1-yl-ethylcarbamoyl)-phenyl]-2-trifluoromethyl-benzamide (77)

MS M/z: 463.2.

### 2-Methyl-N-[3-((S)-1-naphthalen-1-yl-ethylcarbamoyl)-phenyl]-benzamide (78)

MS M/z: 409.3.

### 2-Iodo-N-[3-((S)-1-naphthalen-1-yl-ethylcarbamoyl)-phenyl]-benzamide (79)

MS M/z: 521.1.

### Naphthalene-1-carboxylic acid [3-((S)-1-naphthalen-1-yl-ethylcarbamoyl)-phenyl]-amide (80)

MS M/z: 445.2.

### Naphthalene-2-carboxylic acid [3-((S)-1-naphthalen-1-yl-ethylcarbamoyl)-phenyl]-amide (81)

MS M/z: 445.2.

### Biphenyl-4-carboxylic acid [3-((S)-1-naphthalen-1-yl-ethylcarbamoyl)-phenyl]-amide (82)

MS M/z: 471.2.

### N-[3-((S)-1-Naphthalen-1-yl-ethylcarbamoyl)-phenyl]-2-thiophen-2-yl-benzamide (83)

MS M/z: 477.2.

### 3,4,5-Trimethoxy-N-[3-((S)-1-naphthalen-1-yl-ethylcarbamoyl)-phenyl]-benzamide (84)

MS M/z: 485.3.

### Example 5

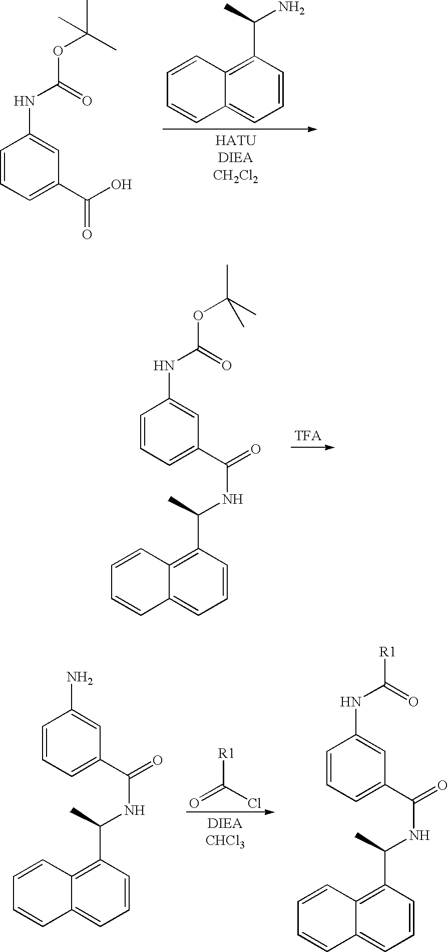

### [3-((R)-1-Naphthalen-1-yl-ethylcarbamoyl)-phenyl]-carbamic acid tert-butyl ester

3-(Boc-amino)benzoic acid (500 mg, 2.11 mmol) was dissolved in CH₂Cl₂ (5 mL), HATU (961 mg, 2.53 mmol) and (R)-1-naphthalen-1-yl-ethylamine (406 μL, 2.53 mmol) was added. To the stirred solution was added N,N-diisopropylethylamine (703 μL, 4.21 mmol). The reaction mixture was stirred overnight. The product precipitated from the reaction mixture. The product was filtered off and washed with CH₂Cl₂ and dried to obtain the title compound in 62% yield. MS M/z: 391.1.

### 3-Amino-N—((R)-1-naphthalen-1-yl-ethyl)-benzamide

[3-((R)-1-Naphthalen-1-yl-ethylcarbamoyl)-phenyl]-carbamic acid tert-butyl ester (510 mg, 1.31 mmol) was dissolved in TFA (50 ml) and stirred for 45 min. The reaction mixture was evaporated. The crude product was used without further purification.

MS M/z: 291.1.

### General Procedure for Compounds 86-101:

3-Amino-N—((R)-1-naphthalen-1-yl-ethyl)benzamide (30 mg, 0.10 mmol) was dissolved in CHCl₃ (2 mL) and N,N-diisopropylethylamine (171 μL, 1.03 mmol) was added. To the stirred solution acid chloride (1.5 equiv., 0.155 mmol) was added and the reaction mixture was stirred overnight. The reaction mixture was evaporated and the crude product was purified on preparative LC/MS (water:acetonitrile 30-80%).

### 2-Chloro-N-[3-((R)-1-naphthalen-1-yl-ethylcarbamoyl)-phenyl]-benzamide (101)

Prepared according to the general procedure using 2-chlorobenzoic acid chloride (27.1 mg, 0.155 mmol). The product was purified on preparative LC/MS (water:acetonitrile 30-80%) to obtain the pure title compound in 15% yield.

NMR ¹H (CDCl₃): δ 8.18 (d, J=8.3 Hz, 1H), 8.11 (s, 1H), 7.86 (m, 2H), 7.82 (d, J=7.9 Hz, 1H), 7.76 (dd, J=7.4, 1.5 Hz, 1H), 7.59 (d, J=7.6 Hz, 1H), 7.48 (m, 4H), 7.40 (m, 4H), 6.42 (d, J=7.9 Hz, 1H), 6.08 (m, 1H), 1.79 (d, J=6.7 Hz, 3H); MS M/z: 429.1.

Similarly, the following compounds were prepared according to the general procedure using the appropriate reagents:

### N-[3-((R)-1-Naphthalen-1-yl-ethylcarbamoyl)-phenyl]-3,5-bis-trifluoromethyl-benzamide (86)

MS M/z: 531.2.

### 2-Bromo-N-[3-((R)-1-naphthalen-1-yl-ethylcarbamoyl)-phenyl]-benzamide (87)

MS M/z: 473.1.

### 2-Fluoro-N-[3-((R)-1-naphthalen-1-yl-ethylcarbamoyl)-phenyl]-benzamide (88)

MS M/z: 413.2.

### 2,6-Dichloro-N-[3-((R)-1-naphthalen-1-yl-ethylcarbamoyl)-phenyl]-benzamide (89)

MS M/z: 465.1.

### Acetic acid 2-[3-((R)-1-naphthalen-1-yl-ethylcarbamoyl)-phenylcarbamoyl]-phenyl ester (90)

MS M/z: 453.2.

### 2-Methoxy-N-[3-(R)-1-naphthalen-1-yl-ethylcarbamoyl)-phenyl]-benzamide (91)

MS M/z: 425.2.

### 2,6-Dimethoxy-N-[3-((R)-1-naphthalen-1-yl-ethylcarbamoyl)-phenyl]-benzamide (92)

MS M/z: 455.3.

### N-[3-((R)-1-Naphthalen-1-yl-ethylcarbamoyl)-phenyl]-2-trifluoromethyl-benzamide (93)

MS M/z: 463.2.

### 2-Methyl-N-[3-((R)-1-naphthalen-1-yl-ethylcarbamoyl)-phenyl]-benzamide (94)

MS M/z: 409.3.

### 3,4,5-Trimethoxy-N-[3-((R)-1-naphthalen-1-yl-ethylcarbamoyl)-phenyl]-benzamide (95)

MS M/z: 485.2.

### 2-Iodo-N-[3-((R)-1-naphthalen-1-A-ethylcarbamoyl)-phenyl]-benzamide (96)

MS M/z: 521.1.

### Naphthalene-1-carboxylic acid [3-((R)-1-naphthalen-1-yl-ethylcarbamoyl)-phenyl]-amide (97)

MS M/z: 445.2.

### Naphthalene-2-carboxylic acid [3-((R)-1-naphthalen-1-yl-ethylcarbamoyl)-phenyl]-amide (98)

MS M/z: 445.2.

### Biphenyl-4-carboxylic acid [3-((R)-1-naphthalen-1-yl-ethylcarbamoyl)-phenyl]-amide (99)

MS M/z: 471.3.

### N-[3-((R)-1-Naphthalen-1-yl-ethylcarbamoyl)-phenyl]-2-thiophen-2-yl-benzamide (100)

MS M/z: 477.1.

### Example 6

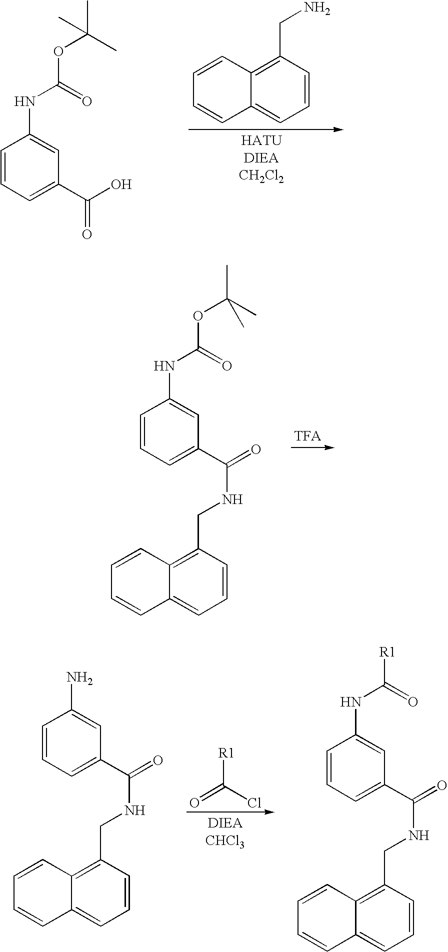

### {3-[(Naphthalen-1-ylmethyl)-carbamoyl]-phenyl}-carbamic acid tent-butyl ester

3-(Boc-amino)benzoic acid (500 mg, 2.11 mmol) was dissolved in CH₂Cl₂ (5 mL), HATU (961 mg, 2.53 mmol) and Naphthalen-1-yl-methylamine (371 μL, 2.53 mmol) was added. To the stirred solution was added N,N-diisopropylethylamine (703 μL, 4.21 mmol). The reaction mixture was stirred overnight. The reaction mixture was washed with water dried and evaporated. The product was purified on column chromatography (isohexane:EtOAc 5:1) to obtain the title compound in 89% yield. MS M/z: 377.1.

### 3-Amino-N-naphthalen-1-ylmethyl-benzamide

{3-[(Naphthalen-1-ylmethyl)-carbamoyl]-phenyl}-carbamic acid tert-butyl ester (690 mg, 1.83 mmol) was dissolved in TFA (50 mL) and stirred for 45 min. The reaction mixture was evaporated. The crude product was used without further purification.

MS M/z: 276.9.

### General Procedure for Compounds 102-117:

3-Amino-N-naphthalen-1-ylmethyl-benzamide (30 mg, 0.11 mmol) was dissolved in CHCl₃ (2 mL) and N,N-diisopropylethylamine (179 μL, 1.09 mmol) was added. To the stirred solution acid chloride (1.5 equiv., 0.163 mmol) was added and the reaction mixture was stirred overnight. The reaction mixture was evaporated and the crude product was purified on preparative LC/MS (water:acetonitrile 30-80%).

### 2-Chloro-N-{3-[(naphthalen-1-ylmethyl)-carbamoyl]-phenyl}-benzamide (117)

Prepared according to the general procedure using 2-chlorobenzoic acid chloride (28.5 mg, 0.163 mmol). The product was purified on preparative LC/MS (water:acetonitrile 30-80%) to obtain the pure title compound in 15% yield.

NMR ¹H (CDCl₃): δ 8.42 (bs, 1H), 8.04 (m, 2H), 7.93 (d, J=7.6 Hz, 1H), 7.89 (d, J=7.6 Hz, 1H), 7.84 (d, J=7.9 Hz, 1H), 7.71 (m, 1H), 7.57-7.31 (m, 9H), 6.44 (m, 1H), 5.01 (d, J=5.3 Hz, 2H); MS M/z: 415.1.

Similarly, the following compounds were prepared according to the general procedure using the appropriate reagents:

### N-{3-[(Naphthalen-1-ylmethyl)-carbamoyl]-phenyl}-3,5-bis-trifluoromethyl-benzamide (102)

MS M/z: 517.2.

### 2-Bromo-N-{3-[(naphthalen-1-ylmethyl)-carbamoyl]-phenyl}-benzamide (103)

MS M/z: 459.1.

### 2-Fluoro-N-{3-[(naphthalen-1-ylmethyl)-carbamoyl]-phenyl}-benzamide (104)

MS M/z: 399.2.

### 2,6-Dichloro-N-{3-[(naphthalen-1-ylmethyl)-carbamoyl]-phenyl}-benzamide (105)

MS M/z: 451.1.

### Acetic acid 2-{3-[(naphthalen-1-ylmethyl)-carbamoyl]-phenylcarbamoyl}-phenyl ester (106)

MS M/z: 439.2.

### 2-Methoxy-N-{3-[(naphthalen-1-ylmethyl)-carbamoyl]-phenyl}-benzamide (107)

MS M/z: 411.2.

### 2,6-Dimethoxy-N-{3-[(naphthalen-1-ylmethyl)-carbamoyl]-phenyl}-benzamide (108)

MS M/z: 441.2.

### N-{3-[(Naphthalen-1-ylmethyl)-carbamoyl]-phenyl}-2-trifluoromethyl-benzamide (109)

MS M/z: 449.2.

### N-{3-[(Naphthalen-1-ylmethyl)-carbamoyl]-phenyl}-2-methyl-benzamide (110)

MS M/z: 395.2.

### 3,4,5-Trimethoxy-N-{3-[(naphthalen-1-ylmethyl)-carbamoyl]-phenyl}-benzamide (111)

MS M/z: 471.2.

### 2-Iodo-N-{3-[(naphthalen-1-ylmethyl)-carbamoyl]-phenyl}-benzamide (112)

MS M/z: 507.1.

### Naphthalene-1-carboxylic acid {3-[(naphthalen-1-ylmethyl)-carbamoyl]-phenyl}-amide (113)

MS M/z: 431.2.

### Naphthalene-2-carboxylic acid {3-[(naphthalen-1-ylmethyl)-carbamoyl]-phenyl}-amide (114)

MS M/z: 431.2.

### Biphenyl-4-carboxylic acid {3-[(naphthalen-1-ylmethyl)-carbamoyl]-phenyl}-amide (115)

MS M/z: 456.2.

### N-{3-[(Naphthalen-1-ylmethyl)-carbamoyl]-phenyl}-2-thiopene-2-yl-benzamide (116)

MS M/z: 463.2.

### Example 7

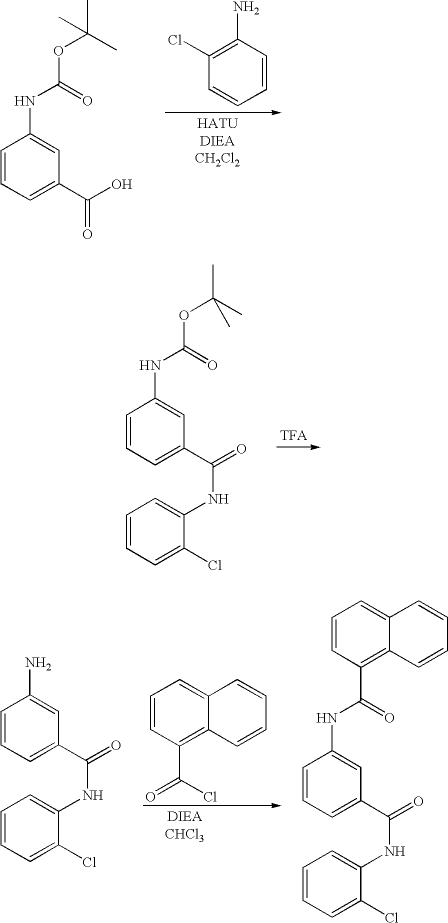

### Naphthalene-1-carboxylic acid [3-(2-chloro-phenylcarbamoyl)-phenyl]-amide (118)

3-(Boc-amino)benzoic acid (300 mg, 1.09 mmol) was dissolved in DMF (5 mL), HATU (497 mg, 1.31 mmol) and 2-chloroaniline (137 μL, 1.31 mmol) was added. To the stirred solution was added N,N-diisopropylethylamine (362 μL, 2.18 mmol). The reaction mixture was stirred for 1 h and then heated to reflux for 10 min. The solvent was evaporated and the product was purified on circular chromatography (isohexane: EtOAc 0-30%) to obtain [3-(2-chloro-phenylcarbamoyl)-phenyl]-carbamic acid tert-butyl ester in. 93% yield. MS M/z: 347.1.

[3-(2-Chloro-phenylcarbamoyl)-phenyl]-carbamic acid tert-butyl ester (300 mg, 0.87 mmol) was dissolved in TFA (50 mL) and stirred for 45 min. The reaction mixture was evaporated. The crude 3-amino-N-(2-chloro-phenyl)-benzamide was used without further purification.

MS M/z: 246.9.

3-Amino-N-(2-chloro-phenyl)-benzamide (267 mg, 1.08 mmol) was dissolved in CHCl₃ (2 mL) and N,N-diisopropylethylamine (171 μL, 1.03 mmol) was added. To the stirred solution 1-naftoyl chloride (326 μL, 2.16 mmol) was added and the reaction mixture was stirred overnight. The reaction mixture was evaporated and the crude product was purified on preparative LC/MS (water:acetonitrile 30-80%) to obtain the title compound in 2% yield.

NMR ¹H (CDCl₃): δ 8.44 (m, 2H), 8.35 (m, 1H), 8.19 (s, 1H), 8.08 (m, 2H), 7.95 (d, J=8.3 Hz, 1H), 7.88 (m, 1H), 7.75 (d, J=7.4 Hz, 1H), 7.65 (d, J=7.4 Hz, 1H), 7.55 (m, 3H), 7.48 (t, J=7.2 Hz, 1H), 7.41 (dd, J=8.1, 1.5 Hz, 1H), 7.28 (m, 1H), 7.08 (td, J=7.4, 1.1 Hz, 1H);

MS M/z: 401.0.

### Example 9

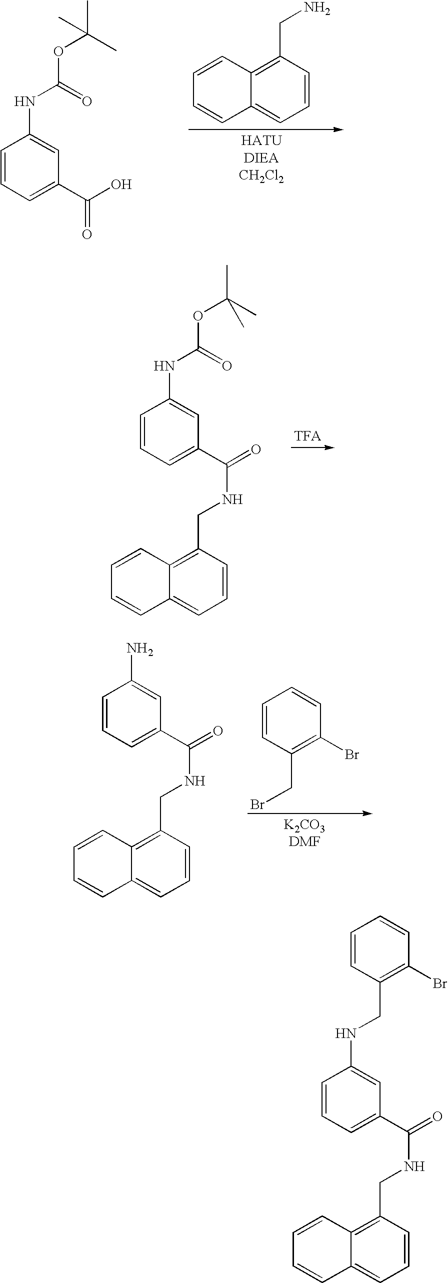

### 3-(2-Bromo-benzylamino)-N-naphthalen-1-ylmethyl-benzamide (122)

3-(Boc-amino)benzoic acid (500 mg, 2.11 mmol) was dissolved in CH₂Cl₂ (5 mL), whereafter HATU (961 mg, 2.53 mmol) and naphthalen-1-yl-methylamine (371 μL, 2.53 mmol) were added. To the stirred solution was added N,N-diisopropylethylamine (703 μL, 4.21 mmol). The reaction mixture was stirred overnight. The reaction mixture was washed with water dried and evaporated. The product was purified by column chromatography (isohexane:EtOAc 5:1) to obtain {3-[(naphthalen-1-ylmethyl)-carbamoyl]-phenyl}-carbamic acid tert-butyl ester in 89% yield. MS M/z: 377.1.

{3-[(Naphthalen-1-ylmethyl)-carbamoyl]-phenyl}-carbamic acid tert-butyl ester (690 mg, 1.83 mmol) was dissolved in TFA (50 mL) and stirred for 45 min. The reaction mixture was evaporated. The crude 3-amino-N-naphthalen-1-ylmethyl-benzamide was used without further purification. MS M/z: 276.9.

3-Amino-N-naphthalen-1-ylmethyl-benzamide (50.0 mg, 0.180 mmol), 2-bromobenzyl bromide (41.9 μL, 0.271 mmol) and K₂CO₃ (125 mg, 0.905 mmol) were dissolved in DMF (2 mL) in a capped microwave reaction tube. The mixture was heated for 8 min at 140° C. Solid material was filtered off and the filtrate was evaporated. The crude product was purified on preparative LC/MS (water:acetonitrile 30-80%) to obtain the title compound in 20% yield.

NMR ¹H (CDCl₃): δ 8.08 (m, 1H), 7.89 (m, 1H), 7.83 (d, J=8.2 Hz, 1H), 7.52 (m, 4H), 7.45 (m, 1H), 7.32 (dd, J=7.5, 1.2 Hz, 1H), 7.21 (dt, J=7.5, 1.2 Hz, 1H), 7.12 (m, 3H), 6.96 (m, 1H), 6.66 (m, 1H), 6.28 (m, 1H), 5.07 (d, J=5.2 Hz, 2H), 4.41 (s, 2H), 4.32 (bs, 1H); MS M/z: 444.9.

### Example 11

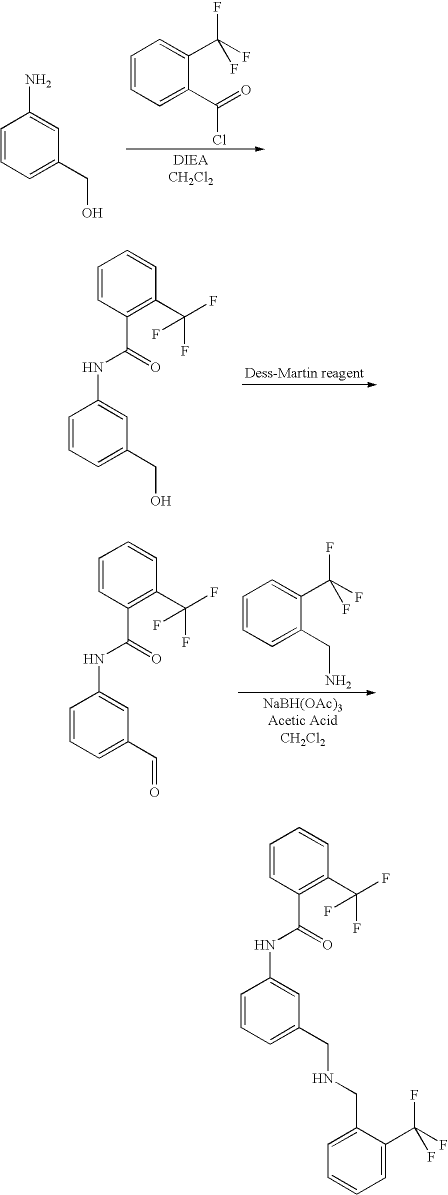

### 2-Trifluoromethyl-N-{3-[(2-trifluoromethyl-benzylamino)-methyl]-phenyl}-benzamide (125)

3-Amino-benzyl alcohol (300 mg, 2.44 mmol) was dissolved in CH₂Cl₂ (10 mL) and 2-trifluoromethylbenzoyl chloride (358 μL, 2.44 mmol) was added. To the stirred solution was added N,N-diisopropylethylamine (1219 μl, 7.31 mmol). The reaction mixture was stirred overnight and then evaporated. The crude N-(3-hydroxymethyl-phenyl)-2-trifluoromethyl-benzamide was used without further purification. MS M/z: 296.1.

N-(3-hydroxymethyl-phenyl)-2-trifluoromethyl-benzamide (230 mg, 0.780 mmol) dissolved in CH₂Cl₂ (10 mL) and cooled slightly. Dess-Martin reagent (1,1,1-triacetoxy-1,1-dihydro-1,2-benziodoxol-3(1H)-one) (347 mg, 0.818 mmol) was added to the stirred solution and the reaction mixture was stirred for 2 h. Na₂S₂O₃ was added to NaHCO₃ (sat. aq.) and the solution was added to the reaction mixture and stirred for 5 min. The organic phase was separated and washed with sat. NaHCO₃, brine, water, dried and evaporated. The crude N-(3-formyl-phenyl)-2-trifluoromethyl-benzamide was used without further purification.

MS M/z: 294.0.

N-(3-Formyl-phenyl)-2-trifluoromethyl-benzamide (220 mg, 0.750 mmol) and 2-trifluoromethylbenzylamine (158 μL, 1.13 mmol) were dissolved in CH₂Cl₂ (10 mL) and one drop of acetic acid was added to the solution. The reaction mixture was stirred for 30 min. and the imine started to precipitate. To the reaction mixture was added NaBH(OAc)₃ (477 mg, 2.25 mmol) and the reaction mixture was stirred for 2 h. The reaction mixture was diluted with CH₂Cl₂ and washed with sat. NaHCO₃ and dried and evaporated. The crude product was purified on preparative LC/MS (water:acetonitrile 30-80%) to obtain the title compound in 12% yield. MS m/z: 453.1.

### Example 12

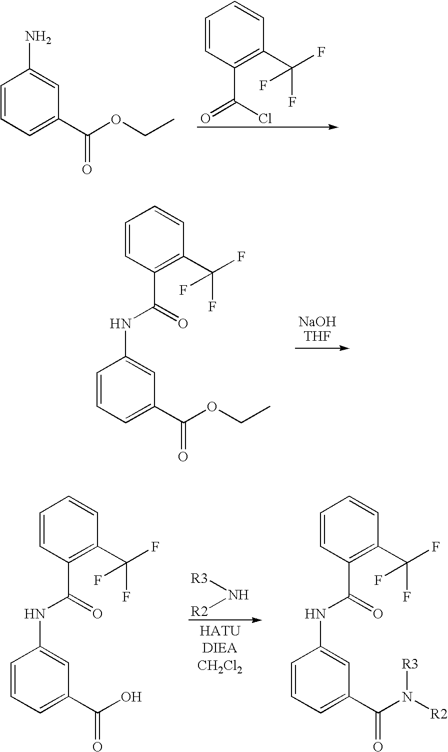

### 3-(2-Trifluoromethyl-benzoylamino)-benzoic acid ethyl ester

Ethyl-3-aminobenzoate (1.00 g, 6.05 mmol) was dissolved in CH₂Cl₂ (30 mL) and 2-trifluoromethylbenzoyl chloride (1.33 mL, 9.08 mmol) was added. To the stirred solution was added N,N-diisopropylethylamine (3.03 mL, 18.2 mmol). The solution was stirred overnight and then extracted with brine. The organic phase was dried and evaporated. The crude product was purified on silica gel with isohexane:EtOAc (85:15) to give the pure product in quantitative yield. MS M/z: 338.0.

### 3-(2-Trifloromethyl-benzoylamino)-benzoic acid

3-(2-Trifluoromethyl-benzoylamino)-benzoic acid ethyl ester (2.00 g, 5.93 mmol) was dissolved in THF (40 mL). NaOH (aq.) (400 mL, 1M) was added to the solution. The reaction mixture was refluxed overnight. The TI-IF was evaporated and the aqueous solution was acidified with conc. HCl. The product starts to precipitate. The acidification was continued until pH approx. 4. The product was filtered off and dried under vacuum (98% yield).

MS M/z: 310.0.

### General Procedure for Compounds 134-149:

3-(2-Trifluoromethyl-benzoylamino)-benzoic acid (20 mg, 0.0647 mmol) was dissolved in CH₂Cl₂ (2 mL), HATU (29.5 mg, 0.0776 mmol) and amine (1.2 equiv., 0.0776 mmol) was added. To the stirred solution was added N,N-diisopropylethylamine (43.1 μL, 0.259 mmol). The reaction mixture was stirred overnight and thereafter evaporated. The crude product was purified on circular chromatography.

### N-(Benzyl)-3-(2-trifluoromethyl-benzoylamino)-benzamide (141)

Prepared according to the general procedure using benzylamine (8.48 μL, 0.0776 mmol). The product was purified on preparative LC/MS (water:acetonitrile 30-80%) to obtain the pure title compound in 84% yield.

NMR ¹H (CDCl₃): δ 8.05 (m, 1H), 7.88 (m, 2H), 7.73 (d, J=7.5 Hz, 1H), 7.64 (m, 2H), 7.58 (m, 2H), 7.43 (t, J=7.9 Hz, 1H), 7.32 (m, 5H), 6.48 (m, 1H), 4.52 (d, J=5.3 Hz, 2H); MS M/z: 399.1.

### N-(2-Methyl-benzyl)-3-(2-trifluoromethyl-benzoylamino)-benzamide (142)

Prepared according to the general procedure using 2-methylbenzylamine (9.63 μL, 0.0776 mmol). The product was purified on preparative LC/MS (water:acetonitrile 30-80%) to obtain the pure title compound in 81% yield. MS M/z: 413.1.

### N-(2-Chloro-benzyl)-3-(2-trifluoromethyl-benzoylamino)-benzamide (143)

Prepared according to the general procedure using 2-chlorobenzylamine (9.40 μL, 0.0776 mmol). The product was purified on preparative LC/MS (water:acetonitrile 30-80%) to obtain the pure title compound in 45% yield. MS M/z: 433.0.

### N-(2-Methoxy-benzyl)-3-(2-trifluoromethyl-benzoylamino)-benzamide (144)

Prepared according to the general procedure using 2-methoxybenzylamine (10.1 μL, 0.0776 mmol). The product was purified on preparative LC/MS (water:acetonitrile 30-80%) to obtain the pure title compound in 78% yield. MS M/z: 429.1.

### N-(2-Dimethylamino-benzyl)-3-(2-trifluoromethyl-benzoylamino)-benzamide (145)

Prepared according to the general procedure using 2-dimethylamino-benzylamine (11.7 mg, 0.0776 mmol). The product was purified on preparative LC/MS (water:acetonitrile 30-80%) to obtain the pure title compound in 22% yield. MS M/z: 442.1.

### N-(2-piperidin-1-yl-benzyl)-3-(2-trifluoromethyl-benzoylamino)-benzamide (146)

Prepared according to the general procedure using 2-piperidin-benzylamine (14.8 μL, 0.0776 mmol). The product was purified on preparative LC/MS (water:acetonitrile 30-80%) to obtain the pure title compound in 61% yield. MS M/z: 482.2.

### N-(2-Trifluoromethoxy-benzyl)-3-(2-trifluoromethyl-benzoylamino)-benzamide (147)

Prepared according to the general procedure using 2-(trifluoromethoxy)-benzylamine (14.8 μL, 0.0776 mmol). The product was purified on preparative LC/MS (water:acetonitrile 30-80%) to obtain the pure title compound in 58% yield.

NMR ¹H (CDCl₃): δ 8.01 (s, 1H), 7.89 (d, J=8.5 Hz, 1H), 7.81 (m, 1H), 7.72 (d, J=8.5 Hz, 1H), 7.64 (m, 2H), 7.59 (m, 1H), 7.52 (m, 1H), 7.45 (m, 2H), 7.35-7.25 (m, 3H), 6.52 (m, 1H), 4.62 (d, J=5.2 Hz, 2H); MS M/z: 483.1.

### N-(2,3-dihydro-benzo[1,4]dioxin-5-ylmethyl)-3-(2-trifluoromethyl-benzoylamino)-benzamide (148)

Prepared according to the general procedure using 2,3-dihydro-1,4-benzodioxin-5-yl-methylamine HCl (15.6 mg, 0.0776 mmol). The product was purified on preparative LC/MS (water:acetonitrile 30-80%) to obtain the pure title compound in 74% yield. MS M/z: 457.1.

### N-(2-morpholin-4-yl-benzyl)-3-(2-trifluoromethyl-benzoylamino)-benzamide (149)

Prepared according to the general procedure using 2-morpholine-benzylamine (14.9 mg, 0.0776 mmol). The product was purified on preparative LC/MS (water:acetonitrile 30-80%) to obtain the pure title compound in 90% yield. MS M/z: 484.2.

Similarly, the following compounds were prepared according to the general procedure using the appropriate reagents:

### 3-(2-Trifluoromethyl-benzoylamino)-N-(2-trifluoromethyl-benzyl)-benzamide (134)

MS M/z: 467.1.

### N-(2-Bromo-benzyl)-3-(2-trifluoromethyl-benzoylamino)-benzamide (135)

MS M/z: 477.

### N-(2-Phenyl-benzyl)-3-(2-trifluoromethyl-benzoylamino)-benzamide (136)

MS M/z: 475.1.

### N-(3-Phenyl-benzyl)-3-(2-trifluoromethyl-benzoylamino)-benzamide (137)

MS M/z: 475.1.

### N-(3-Bromo-benzyl)-3-(2-trifluoromethyl-benzoylamino)-benzamide (138)

MS M/z: 479.27.

### N-(2,3-Dimethyl-benzyl)-3-(2-trifluoromethyl-benzoylamino)-benzamide (139)

MS M/z: 427.2.

### N-(8-hydroxy-quinolin-2-yl)-3-(2-trifluoromethyl-benzoylamino)-benzamide (140)

MS M/z: 452.1.

### General Procedure for Compounds 151-160.

3-(2-Trifluoromethyl-benzoylamino)benzoic acid (20 mg, 0.0647 mmol) was dissolved in CH₂Cl₂ (2 mL), HATU (29.5 mg, 0.0776 mmol) and amine (3 equiv., 0.194 mmol) was added. To the stirred solution was added N,N-diisopropylethylamine (43.1 μL, 0.259 mmol). The reaction mixture was stirred for 72 hours at 40° C. and thereafter concentrated in vacuo. The crude product was purified as below.

### 3-[2-(Trifluoromethyl)-benzoylamino]-N-(3-bromo-pyridine-2-yl)-benzamide (151)

Prepared according to the general procedure using 2-amino-3-bromopyridine (33.6 mg, 0.194 mmol). The product was purified on preparative LC/MS (water:acetonitrile 30-80%) to obtain the pure title compound in 16% yield. MS M/z: 465.9 (M+1).

### 3-[2-(Trifluoromethyl)-benzoylamino]-N-(5-methyl-pyridine-2-yl)-benzamide (152)

Prepared according to the general procedure using 6-amino-3-picolin (21.0 mg, 0.194 mmol). The product was purified on preparative LC/MS (water:acetonitrile 30-80%) to obtain the pure title compound in 60% yield. MS M/z: 400.0 (M+1).

### 3-[2-(Trifluoromethyl)-benzoylamino]-N-(5-trifluoromethyl-pyridine-2-yl)-benzamide (153)

Prepared according to the general procedure using 5-(trifluoromethyl)pyridine-2-amine (31.5 mg, 0.194 mmol). The product was purified on preparative LC/MS (gradient elution water:acetonitrile 30-80%) to obtain the pure title compound in 19% yield. MS M/z: 454.0 (M+1).

### 3-[2-(Trifluoromethyl)-benzoylamino]-N-(5-cyano-pyridine-2-yl)-benzamide (154)

Prepared according to the general procedure using 2-amino-5-cyanopyridine (23.1 mg, 0.194 mmol). The product was purified on preparative LC/MS (water:acetonitrile 30-80%) to obtain the pure title compound in 6% yield. MS M/z: 411.0 (M+1).

### 3-[2-(Trifluoromethyl)-benzoylamino]-N-(5-bromo-pyridine-2-yl)-benzamide (155)

Prepared according to the general procedure using 2-amino-5-bromopyridine (33.6 mg, 0.194 mmol). The product was purified on preparative LC/MS (water:acetonitrile 30-80%) to obtain the pure title compound in 19% yield. MS M/z: 463.9 (M+1).

### 3-[2-(Trifluoromethyl)-benzoylamino]-N-(5-fluoro-pyridine-2-yl)-benzamide (156)

Prepared according to the general procedure using 2-amino-5-fluoropyridine (21.8 mg, 0.194 mmol). The product was purified on preparative LC/MS (water:acetonitrile 30-80%) to obtain the pure title compound in 43% yield. MS M/z: 404.0 (M+1).

### 3-[2-(Trifluoromethyl)-benzoylamino]-N-(5-chloro-pyridine-2-yl)-benzamide (157)

Prepared according to the general procedure using 2-amino-5-chloropyridine (24.9 mg, 0.194 mmol). The product was purified on preparative LC/MS (water:acetonitrile 30-80%) to obtain the pure title compound in 32% yield. MS M/z: 419.9 (M+1).

### 3-[2-(Trifluoromethyl)-benzoylamino]-N-(5-iodo-pyridine-2-yl)-benzamide (158)

Prepared according to the general procedure using 2-amino-5-iodopyridine. The product was purified with column chromatography (Silica gel) followed by recrystallization to obtain the pure title compound in 16% yield. MS m/z: 511.9 (M+1).

### 3-[2-(Trifluoromethyl)-benzoylamino]-N-(5-bromo-6-methylpyridine-2-yl)-benzamide (159)

Prepared according to the general procedure using 5-bromo-6-methylpyridin-2-amine. The product was purified with column chromatography (Silica gel) followed by recrystallization to obtain the pure title compound in 22% yield. MS m/z: 479.9 (M+1).

### 3-[2-(Trifluoromethyl)-benzoylamino]-N-(4,6-dimethyl-pyridine-2-yl)-benzamide (160)

Prepared according to the general procedure using 4,6-dimethylpyridin-2-amine. The product was purified with column chromatography (Silica gel) followed by recrystallization to obtain the pure title compound in 20% yield. MS m/z: 414.1 (M+1).

### General Procedure for the Preparation of Compounds 161-164

3-({[2-(Trifluoromethyl)phenyl]carbonyl}amino)benzoic acid (31 mg, 0.10 mmol), HATU (N-[[(dimethyl-amino)-1H-1,2,3-triazolo[4,5-b]-pyridin-1-yl]methylene]-N-methyl-methanaminium hexafluorophosphate N-oxide, 46 mg, 0.12 mM), TMP (2,4,6-trimethylpyridine, 48.4 mg, 0.40 mmol) and the appropriate amine (0.40 mmol) were mixed in dichloromethane. The reaction mixture was stirred in a microwave oven for 0.5 h at 140° C. The reaction mixture was filtered on a pad of silica (eluent: n-hexane/ethyl acetate 1:1) and finally purified on a preparative LC-MS (reverse-phase column, gradient elution using the following eluents: A=water/HCOOH 99.95:0.05 and B=acetonitrile/HCOOH 99.95:0.05. Elution started at the proportions A/B 9:1, and ended at A/B 1:7).

### N-(3-(benzo[d]thiazol-5-ylcarbamoyl)phenyl)-2-(trifluoromethyl)benzamide (161)

This Example was prepared and purified according to the general procedure above, using benzo[d]thiazol-5-amine (60 mg, 0.40 mmol), to give 26% of the title compound. MS m/z: 442.1 (M+1).

### N-(3-(2-methylbenzo[d]thiazol-6-ylcarbamoyl)phenyl)-2-(trifluoromethyl)benzamide (162)

This Example was prepared and purified according to the general procedure above, using 2-methylbenzo[d]thiazol-6-amine (66 mg, 0.40 mmol), to give 30% of the title compound. MS m/z: 456.1 (M+1).

### N-(3-(2-methylbenzo[d]oxazol-6-ylcarbamoyl)phenyl)-2-(trifluoromethyl)benzamide (163)

This Example was prepared and purified according to the general procedure above, using 2-methylbenzo[d]oxazol-6-amine (59 mg, 0.40 mmol), to give 22% of the title compound. MS m/z: 440.1 (M+1).

### N-(3-(5-chlorobenzo[d]oxazol-2-ylcarbamoyl)phenyl)-2-(trifluoromethyl)benzamide (164)

This Example was prepared and purified according to the general procedure above, using 5-chlorobenzo[d]oxazol-2-amine (67 mg, 0.40 mmol), to give 8.06 mg (25%) of the title compound. MS m/z: 460.1 (M+1).

### Examples 165-167

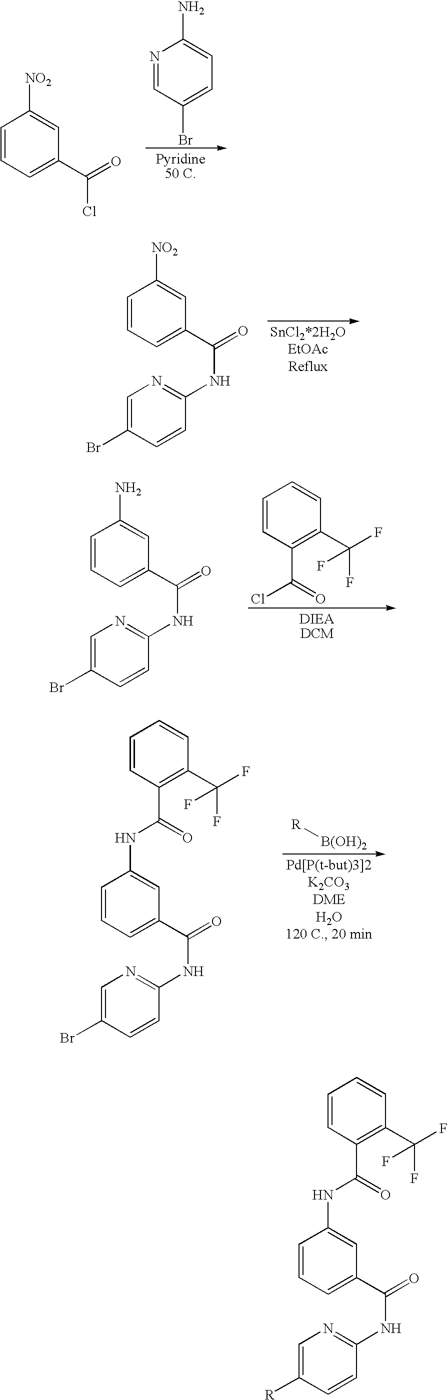

### N-(5-Bromo-pyridin-2-yl)-3-nitro-benzamide

A test tube with screw cap was charged with 3-nitrobenzoyl chloride (1.00 g, 5.39 mmol) and 2-amino-5-bromopyridine (932 mg, 5.39 mmol). Pyridine (15 mL) was added as solvent and base. The reaction mixture was stirred at 50° C. for 3 days. The reactants were not dissolved directly, but after 3 days the solution had turned to a clear pail yellow solution with a salt precipitation on the top. The reaction was monitored by LCMS and the product formation was confirmed. The reaction mixture was evaporated and diluted with EtOAc (the salts did not dissolve in EtOAc) and washed with saturated NaHCO₃ three times. The water phase was extracted with EtOAc three times. The combined organic phase was dried with MgSO4 and concentrated in vacuo. The residue was used in the next step without further purification. Dry weight: 1.75 g. MS m/z: 322.0, 324.0.

### 3-Amino-N-(5-bromo-pyridin-2-yl)-benzamide

To a round flask with N-(5-bromo-pyridin-2-yl)-3-nitro-benzamide (1.75 g, 5.46 mmol) was added EtOAc (100 mL) and SnCl₂x2H₂O (7.4 g, 33 mmol). The reaction mixture was heated to reflux and stirred for 2 h. The solution was diluted with water and the pH was adjusted to 8. The reaction mixture was extracted with EtOAc and the combined organic phase was washed with brine. The organic phase was dried and concentrated in vacuo. The residue was used in the next step without further purification. Dry weight: 882 mg. MS M/z: 292.0, 294.0.

### 3-[2-(Trifluoromethyl)-benzylamino]-N-(5-bromo-pyridine-2-yl)-benzamide

To a round flask with 3-amino-N-(5-bromo-pyridin-2-yl)-benzamide (881.9 mg, 3.02 mmol) was added dichloromethane (70 mL). To the stirred solution was added 2-(trifluoromethyl)-benzoyl chloride (887 μL, 6.04 mmol) and DIEA (1.51 mL, 9.06 mmol). The reaction was stirred at room temperature overnight. After 20 min the product started to precipitate. The reaction mixture was evaporated and AcCN (˜100 mL) was added, and the solution was heated. The slurry was cooled in room temperature and then in the freezer for 30 min. The product was filtered of and washed with EtOAc and isohexan. The white crystals were pure product according to LCMS. Dry weight: 1301 mg. Yield: 93%. MS M/z: 464.1, 464.1.

### General Procedure for Compounds 165-167:

A microwave tube was charged with 3-[2-(trifluoromethyl)-benzylamino]-N-(5-bromo-pyridine-2-yl)-benzamide (20 mg, 43 μmol), arylboronic acid (3 equiv., 129 μmol), Pd[P(t-bu)₃]₂ (2.2 mg, 4.3 μmol), K₂CO₃ (17.9 mg, 129 μmol), in DME/water (0.5/0.2 mL). The reaction was heated to 130° C. for 20 min in the microwave cavity. The reaction mixture was diluted with DMSO, heated to a clear solution and purified on LCMS.

### 3-[2-(Trifluoromethyl)-benzylamino]-N-(5-thiophene-3-yl-pyridine-2-yl)-benzamide (165)

Prepared according to the general procedure using 3-thiopheneboronic acid (16.5 mg, 129 μmol). The product was purified on preparative LC/MS (water:acetonitrile 30-80%) to obtain the pure title compound in 58% yield. MS M/z: 468.1

### 3-[2-(Trifluoromethyl)-benzylamino]-N-(5-phenyl-pyridine-2-yl)-benzamide (166)

Prepared according to the general procedure using phenylboronic acid (15.8 mg, 129 μmol). The product was purified on preparative LC/MS (water:acetonitrile 30-80%) to obtain the pure title compound in 52% yield. MS M/z: 462.2

### 3-[2-(Trifluoromethyl)-benzylamino]-N-[5-(4-dimethylaminophenyl)-pyridine-2-yl]-benzamide (167)

Prepared according to the general procedure using 4-(dimethylamino)-phenylboronic acid (21.3 mg, 129 μmol). The product was purified on preparative LC/MS (water:acetonitrile 30-80%) to obtain the pure title compound in 42% yield. MS M/z: 505.2

### Biological Tests

Prostaglandins detection kits were purchased from Cayman Chemicals and used according to the instruction of the manufacturer. In vitro toxicology assay kit, MTT based from Sigma, cat N-TOX1.

### HPLC Assay

Earlier studies have demonstrated that prostaglandins can be separated by RP-HPLC and detected by UV spectrophotometry (Terragno et al. Prostaglandins 21(1), 101-12 (1981); Powell Anal. Biochem. 148(1), 59-69 (1985)). The molar extinction coefficient of PGE2 is 16,500 at 192.5 nm (Terragno et al. Prostaglandins 21(1), 101-12 (1981)). The main products of PGH2 are PGF2α, PGE₂ and PGD₂. Using the described RP-HPLC conditions, the retention times were 19.0, 23.8 and 28.6 minutes for PGF2α, PGE₂ and PGD₂, respectively. 11β-PGE₂ was used as the internal standard and 11β-PGE₂ was eluted with a retention time of 25.3 min with almost baseline separation from PGE₂. In order to quantify PGE₂, a standard curve of PGE₂ was made. The curve was linear over the range from 0.9 μmol to 706 μmol (R²=0.9997, k=0.0012). For quantification we routinely use both the external standard as well as the internal standard technique, the latter method accounting also for losses during preparation.

Care must be taken when assaying PGE synthase with PGH₂. The substrate is very labile and decomposes non-enzymatically, with a half-life of about 5 min at 37° C., into a mixture of PGE₂ and PGD₂ with an E/D ratio of about 3. Also, the PGE synthase catalysis is very fast, which is why substrate depletion easily can occur within seconds thus preventing a quantitative analysis. After the reaction has been terminated, any remaining PGH₂ must also rapidly be separated from the products in order not to interfere with the results. In order to minimize non-enzymatic production of PGE₂, the substrate (PGH₂) was always kept on CO₂-ice (−78° C.) until use and the enzyme reaction was performed at 0° C. in the presence of PGH₂ and reduced glutathione (GSH). A stop-solution was used, containing FeCl₂, which converted any remaining PGH₂ into HHT. Also, the products are much more stable in organic solvents so we immediately extracted the sample after termination by solid phase extraction and kept the eluate in acetonitrile.

Protein samples were diluted in potassium inorganic phosphate buffer (0.1M, pH 7.4) containing 2.5 mM reduced glutathione (GSH). 4 μl PGH₂, dissolved in acetone (0.284 mM) were added to Eppendorf tubes and kept on CO₂-ice (−78° C.). Prior to the incubation, both the substrate and samples were transferred onto wet-ice (or 37° C.) for 2 min temperature equilibration. The reaction was started by the addition of the 100 μl sample to the tubes containing PGH₂. The reaction was terminated by the addition of 400 μl stop solution (25 mM FeCl₂, 50 mM citric acid and 2.7 μM 11-β PGE₂), lowering the pH to 3, giving a total concentration of 20 mM FeCl₂, 40 mM citric acid and 2.1 μM 11-β PGE₂. Solid phase extraction was performed immediately using C18-chromabond columns. The samples were eluted with 500 μl acetonitrile and thereafter 1 ml H₂O was added. In order to determine the formation of PGE₂ and 11.43 PGE₂, an aliquot (150 μl) was analyzed by RP-HPLC, combined with UV detection at 195 nm. The reverse-phase HPLC column was Nova-Pak C18 (3.9×150 mm, 4 μm particle size) obtained from Waters and the mobile phase was water, acetonitrile and trifluoroacetic acid (72:28:0.007, by volume). The flow rate was 0.7 ml/min and the products were quantified by integration of the peak areas.

### Thiobarbituric Acid Assay (TBA-MDA Assay or Malondialdehyde Assay)

Malondialdehyde is a product of lipid peroxidation and reacts with thiobarbituric acid forming a red product that absorbs at 535 nm (W. G. Niehaus, Jr and B. Samuelsson, Eur. J. Biochem 6, 126 (1968). The extinction coefficient of the TBA-MDA conjugate is 1.56×10⁵ M⁻¹ cm⁻¹ (E. D. Wills. Biochem. J. 113, 315 (1969).

The method used for detection of inhibition of mPGES-1 is based on the detection of the amount of remaining PGH2. This method was described more than 20 years ago by Basevich et al (Bioorg. Khim. 1983, 9(5), 658-665.

The assay used was a modified variant and used citric acid instead of the TCA-TBA-HCl reagent described in the original assay. In this assay recombinant, membrane-bound mPGES-1 was incubated with PGH2. The reaction was stopped by adding citric acid with a final pH of 3 and a large excess of FeCl2 (20 mM) to convert any remaining PGH2 into MDA and 12-BHT. TBA reagent was finally added (0.67%) and the samples were heated at 80° C. for 30 min. The absorbance of the conjugate was measured at 535 nm.

The product of mPGES-1 (PGE2) was not measured directly in this assay, but rather the remaining substrate (PGH2) indirectly by adding FeCl₂ that converts PGH2 into MLA and 12-HHT. As a positive control a known mPGES-1 inhibitor, MK-886, was used and the new inhibitors were compared with the inhibition of MK-886 (% of MK-886 inhibition).

Red product ˜530 nM (1.56×10⁵M⁻¹ cm⁻¹).

$$Total Activity = \frac{{A}_{530} - {A}_{560} / 1 min}{1.56 \times {10}^{5}} \times \frac{0.265}{0.05} ( U \text{/} ml )$$

Selected Results from the TBA-MDA Assay (1050 in μM)

| Compound No. | IC50 μM |
|---|---|
| 2 | 5.8 |
| 3 | 1.1 |
| 4 | 3.9 |
| 6 | 0.3 |
| 8 | 0.8 |
| 9 | 0.6 |
| 12 | 1.3 |
| 13 | 1.3 |
| 14 | 0.9 |
| 17 | 3.5 |
| 29 | 4.2 |
| 34 | 0.72 |
| 35 | 0.86 |
| 36 | 0.33 |
| 38 | 0.14 |
| 40 | 0.8 |
| 41 | 3.7 |
| 42 | 3.1 |
| 43 | 0.19 |
| 44 | 0.65 |
| 46 | 0.29 |
| 49 | 1.2 |
| 50 | 3.0 |
| 52 | 6.9 |
| 53 | 0.50 |
| 54 | 1.2 |
| 55 | 0.63 |
| 56 | 0.66 |
| 57 | 0.39 |
| 63 | 0.46 |
| 65 | 6.9 |
| 66 | 9.9 |
| 68 | 0.18 |
| 69 | 0.26 |
| 72 | 0.23 |
| 73 | 1.8 |
| 74 | 0.47 |
| 76 | 4.1 |
| 77 | 0.29 |
| 78 | 0.69 |
| 79 | 0.20 |
| 85 | 0.37 |
| 87 | 0.38 |
| 88 | 2.3 |
| 89 | 0.60 |
| 92 | 1.3 |
| 93 | 0.12 |
| 96 | 0.23 |
| 101 | 0.29 |
| 103 | 0.18 |
| 104 | 1.1 |
| 105 | 0.52 |
| 108 | 2.2 |
| 109 | 0.13 |
| 110 | 0.33 |
| 112 | 0.22 |
| 116 | 2.5 |
| 117 | 0.26 |
| 120 | 0.32 |
| 122 | 1.0 |
| 123 | 1.6 |
| 125 | 1.8 |
| 134 | 0.070 |
| 135 | 0.065 |
| 136 | 0.20 |
| 137 | 0.32 |
| 138 | 0.16 |
| 139 | 0.075 |
| 140 | 1.9 |
| 141 | 0.28 |
| 142 | 1.0 |
| 143 | 0.22 |
| 144 | 0.84 |
| 145 | 0.47 |
| 146 | 0.17 |
| 147 | 0.11 |
| 148 | 0.99 |
| 149 | 2.0 |
| 153 | 0.049 |
| 164 | 0.068 |
| 166 | 0.053 |

### Fibroblast Assay

Synovial fibroblasts from human RA patients (passage four) growing in 96 well tissue culture plates were induced with IL-1beta (10 ng/ml) and TNFalfa (10 ng/ml). Test compound at a concentration of 10, 1, 0.1 or 0 μM was added and the cells were further cultured for 24 h.

After 24 hours, supernatants were collected and number of viable cells was evaluated using MTT test according to manufacturer's instructions. PGE₂ levels in supernatants were measured by EIA according to manufacturer's instructions. Results were expressed as PGE2 levels in supernatants (and adjusted for MTT) and related to PGE₂ levels in supernatants from cells which were induced without adding test compound. Since the test compound did not affect cell viability at any concentration tested, normalization for MTT did not contribute to observed differences in PGE₂ content.

### Results

| | PGE₂ % DMSO control | | |
|---|---|---|---|
| | 0.1 μM | 1 μM | 10 μM |
| Ex No. | test comp. | test comp. | test comp. |
| 1 | 43 | 27 | 2 |
| 150 | 59 | 54 | 11 |

### A549 Assay

A549 lung carcinoma cells, seeded at a density of 10,000 cells/well, were grown in 96 well tissue culture plates. TNFalfa (5 ng/ml) and EL-1beta (5 ng/ml) were added and the cells were incubated for 16 hours. Cells were washed in PBS and test compounds at the appropriate concentration in HBSS/0.1% BSA were added. After 30 minutes incubation with test compounds, 10 μM arachidonic acid was added and cells were further incubated for 30 minutes. Supernatant was collected and analyzed for PGE₂ content by EIA according to manufacturer's instructions.

| Ex. No. | 8 | 34 | 77 | 79 | 93 | 104 | 117 | 153 |
|---|---|---|---|---|---|---|---|---|
| EC50 PGE2, μM | 2.96 | 5.4 | 0.4 | 1.23 | 0.45 | 0.64 | 1.48 | 0.50 |

### In-Vivo Results

### LPS Air Pouch Model of Acute Inflammation in the Rat

(Adapted from “Models of Inflammation: Carrageenan Air Pouch in the Rat” current protocols in pharmacology (1998) 5.6.1-5.6.6)

8-12 weeks old Dark Agouti rats were anesthetized with isofluorane and 20 ml of sterile filtered air were injected to each rat subcutaneously into the intracapsular area of the back (20 μm sterile syringe filter, 20 ml syringe, 23-G/1-in needle). The air pouches were allowed to mature for 24 h.

On the day of the experiment, rats were anesthetized and injected intra-peritoneally with 1 mL test compound (ex. No. 70) dissolved in 90% PEG400/10% DMSO resulting in a 75 mg/kg dose of test compound or PEG/DMSO vehicle alone. 30 minutes post administration of test compound or vehicle, an intra-pouch injection with 2 ml of a solution of LPS 5 μg/ml in sterile PBS (2 ml syringe, 20G/1-in needle) was made.

6 h post-LPS injection the rat was killed by CO₂ inhalation. A second 2 ml intra-pouch injection of lavage solution (5.4 mM EDTA in sterile PBS, freshly prepared from a 54 mM EDTA sterile filtered stock solution) (10 ml syringe, 18G/1.5 in needle) was given. The pouch was immediately drained of lavage fluid and the effect of test compound relative vehicle control on the inflammatory reaction was assessed by analyzing PGE2 content in the pouch exudate. PGE2 was measured by EIA (Cayman Chemicals) according to the manufacturer's instructions.

Performing this test on compound (ex. No. 1) resulted in a 35% reduction in inducible PGE2 formation relative to vehicle control.

### Adjuvant-Induced Arthritis

Lewis-derived male rats weighing 205±15 g were used. Test compound (ex No. 1) at 100 mg/kg was administered intra-peritoneally for 5 consecutive days. A well-ground suspension of killed Mycobacterium tuberculosis (0.3 mg in 0.1 mL of light mineral oil; Complete Freund's Adjuvant, CFA) was administered in a single dose into the subplantar region of the rift hind paw 1 hour following the first dose of test substance (denoted day 1). Right hind paw volume was measured by plethysmometer and water cell (25 mm Diameter) on day 0 (before CFA treatment), and on days 1, 5, 8, 11, 14 and 18 after CFA treatment of right paw (with CFA). For CFA-injected right paw volume, the paw volume on days 1, 5, 8, 11, 14 and 18 was compared to that on day 0.

Examples of pharmaceutically acceptable addition salts for use in the pharmaceutical compositions of the present invention include those derived from mineral acids, such as hydrochloric, hydrobromic, phosphoric, metaphosphoric, nitric and sulphuric acids, and organic acids, such as tartaric, acetic, citric, malic, lactic, fumaric, benzoic, glycolic, gluconic, succinic, and arylsulphonic acids. The pharmaceutically acceptable excipients described herein, for example, vehicles, adjuvants, carriers or diluents, are well-known to those who are skilled in the art and are readily available to the public. The pharmaceutically acceptable carrier may be one that is chemically inert to the active compounds and that has no detrimental side effects or toxicity under the conditions of use. Pharmaceutical formulations are found e.g. in Remington: The Science and Practice of Pharmacy, 19th ed., Mack Printing Company, Easton, Pa. (1995).

Prodrugs of the compounds of formula (I) may be prepared by modifying functional groups present on the compound in such a way that the modifications are cleaved, in vivo when such prodrug is administered to a mammalian subject. The modifications typically are achieved by synthesizing the parent compound with a prodrug substituent. Prodrugs include compounds of formula (I) wherein a hydroxy, amino, sulfhydryl, carboxy or carbonyl group in a compound of formula (I) is bonded to any group that may be cleaved in vivo to regenerate the free hydroxyl, amino, or sulfhydryl group, respectively. Examples of prodrugs include, but are not limited to, esters and carbamates of hydroxy functional groups, esters groups of carboxyl functional groups, N-acyl derivatives, N-Mannich bases. General information on prodrugs may be found e.g. in Bundegaard, H. “Design of Prodrugs” pI-92, Elesevier, New York-Oxford (1985).

The composition according to the invention may be prepared for any route of administration, e.g. oral, intravenous, cutaneous or subcutaneous, nasal, intramuscular, or intraperitoneal. The precise nature of the carrier or other material will depend on the route of administration. For a parenteral administration, a parenterally acceptable aqueous solution is employed, which is pyrogen free and has requisite pH, isotonicity and stability. Those skilled in the art are well able to prepare suitable solutions and numerous methods are described in the literature. A brief review of methods of drug delivery is also found in e.g. Langer, Science 249:1527-1533 (1990).

The dose administered to a mammal, particularly a human, in the context of the present invention should be sufficient to affect a therapeutic response in the mammal over a reasonable time frame. One skilled in the art will recognize that dosage will depend upon a variety of factors including the potency of the specific compound, the age, condition and body weight of the patient, as well as the stage/severity of the disease. The dose will also be determined by the route (administration form) timing and frequency of administration. In the case of oral administration the dosage can vary from about 0.01 mg to about 1000 mg per day of a compound of formula (I) or the corresponding amount of a pharmaceutically acceptable salt thereof.

The compounds of the present invention may be used or administered in combination with one or more additional drugs useful in the treatment of pain, fever, inflammations and cancer. The components may be in the same formulation or in separate formulations for administration simultaneously or sequentially. The compounds of the present invention may also be used or administered in combination with other treatment such as irradiation for the treatment of cancer.

1-35. (canceled)

36. A compound of formula (II) 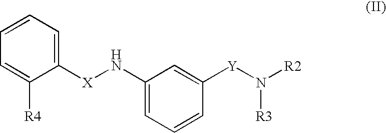
wherein
R² and R³ are each independently selected from H and a moiety selected from alkyl, monocyclic or bicyclic carbocyclyl and monocyclic or bicyclic heterocyclyl, which moiety optionally is substituted with one or more R⁵;
R⁴ is CF₃ or Cl;
each R⁵ is independently selected from halogen, alkyl, monocyclic or bicyclic carbocyclyl, monocyclic or bicyclic heterocyclyl, alkoxy, CN, CF₃, OH, NO₂, COOH, C(O)Oalkyl, OC(O)alkyl, wherein any cyclyl moiety may be substituted with one or more R⁶,
each R⁶ is independently selected from CF₃, halogen, alkyl, alkoxy, CF₃O, N,N-dialkylamino, phenyl, and saturated monocyclic heterocyclyl,
wherein in any of R², R³, R⁵ and R⁶, any cyclyl comprises 1-10 C and any alkyl and alkoxy comprises 1-6 C;
Y and X are independently selected from >CO and >CH₂;
as well as pharmaceutically acceptable salts thereof;
with the proviso that the compound is not 3-(2-chloro-benzoylamino)-N-naphtalene-1-yl-benzamide, 3-(2-chloro-benzoylamino)-N-(2,6-dimethyl-4-(thiophen-2-yl)phenyl)-benzamide, N-[3-[[[2,4-dibromo-6-(trifluoromethyl)phenyl]amino]carbonyl]phenyl]-(trifluoromethyl)-benzamide, 2-chloro-N-[3-[[(2-chlorophenyl)amino]carbonyl]phenyl-benzamide, or 2-chloro-N-[3-[[(2,3-dihydro-1,5-dimethyl-3-oxo-2-phenyl-1H-pyrazol-4-yl)amino]carbonyl]phenyl]-benzamide.

37. A compound according to claim 36, wherein R² and R³ are each independently selected from H and a moiety selected from C1-C6-alkyl, carbocyclyl and heterocyclyl, wherein any cyclyl is 5-7 membered monocyclic or is bicyclic having a 5- or 6-membered cycle fused with a 6-membered cycle.

38. A compound according to claim 36, wherein R² and R³ are each independently selected from H and a moiety selected from methyl, n-butyl, cyclohexyl, cycloheptyl, phenyl, pyridyl, quinolinyl, isoquinolinyl, benzimidazolyl, benzothiazolyl, 1H-indazolyl, indanyl and 1,2,3,4-tetrahydronaphthyl, which moiety is optionally substituted with one or more R⁵ as defined in claim 36.

39. A compound according to claim 36, wherein R³ is selected from H and a moiety selected from C1-C3 alkyl and monocyclic C5-C7 carbocyclyl, which moiety optionally is substituted with one or more R⁵ selected from monocyclic C5-C7 carbocyclyl.

40. A compound according to claim 36, wherein R³ is H.

41. A compound according to claim 36, wherein R⁴ is CF₃.

42. A compound according to claim 36, wherein X and Y are both >CO.

43. A compound according to claim 36, wherein each R⁵ is independently selected from halogen, alkyl, CN, CF₃, OH, and monocyclic or bicyclic carbocyclyl and monocyclic or bicyclic heterocyclyl, wherein any cyclyl moiety may be substituted with one or more R⁶ as defined in claim 36.

44. A compound according to claim 36, selected from
3-(2-Chloro-benzoylamino)-N-benzyl-benzamide,
3-(2-Chloro-benzoylamino)-N-(2-methyl-quinolin-4-yl)-benzamide,
3-(2-Chloro-benzoylamino)-N-butyl-benzamide,
3-(2-Chloro-benzoylamino)-N,N-dibenzyl-benzamide,
3-(2-Chloro-benzoylamino)-N-(diphenylyl-methyl)-benzamide,
3-(2-Chloro-benzoylamino)-N-(isoquinolin-5-yl-)-benzamide,
3-(2-Chloro-benzoylamino)-N-(quinolin-8-yl-)-benzamide,
3-(2-Chloro-benzoylamino)-N-(quinolin-2-yl-)-benzamide,
3-(2-Chloro-benzoylamino)-N-(pyridine-2-ylmethyl)-benzamide,
3-(2-Chloro-benzoylamino)-N-(pyridine-3-ylmethyl)-benzamide,
3-(2-Chloro-benzoylamino)-N-(pyridine-2-yl)-benzamide,
3-(2-Chloro-benzoylamino)-N-(1H-benzimidazol-2-yl)-benzamide,
3-(2-Chloro-benzoylamino)-N-(benzothiazol-6-yl)-benzamide,
3-(2-Chloro-benzoylamino)-N-(1H-indazol-5-yl)-benzamide,
3-(2-Chloro-benzoylamino)-N,N-dicyclohexyl-benzamide,
3-(2-Chloro-benzoylamino)-N-indan-1-yl-benzamide,
3-(2-Chloro-benzoylamino)-N-(1,2,3,4-tetrahydro-naphthalen-1-yl)-benzamide,
3-(2-Chloro-benzoylamino)-N-cycloheptyl-benzamide,
2-Chloro-N-[3-((S)-1-naphthalen-1-yl-ethylcarbamoyl)-phenyl]-benzamide,
2-Chloro-N-[3-((R)-1-naphthalen-1-yl-ethylcarbamoyl)-phenyl]-benzamide,
2-Chloro-N-{3-[(naphthalen-1-ylmethyl)-carbamoyl]-phenyl}-benzamide,
3-(2-Trifluoromethyl-benzoylamino)-N-quinolin-2-yl-benzamide,
N-(3-Cycloheptylcarbamoyl-phenyl)-2-trifluoromethylbenzamide,
N-[3-((S)-1-Naphthalen-1-yl-ethylcarbamoyl)-phenyl]-2-trifluoromethyl-benzamide,
N-[3-((R)-1-Naphthalen-1-yl-ethylcarbamoyl)-phenyl]-2-trifluoromethyl-benzamide,
N-{3-[(Naphthalen-1-ylmethyl)-carbamoyl]-phenyl}-2-trifluoromethyl-benzamide,
2-Trifluoromethyl-N-{3-[(2-trifluoromethyl-benzylamino)-methyl]-phenyl}-benzamide,
3-(2-Trifluoromethyl-benzoylamino)-N-(2-trifluoromethyl-benzyl)-benzamide,
N-(2-Bromo-benzyl)-3-(2-trifluoromethyl-benzoylamino)-benzamide,
N-(2-Phenyl-benzyl)-3-(2-trifluoromethyl-benzoylamino)-benzamide,
N-(3-Phenyl-benzyl)-3-(2-trifluoromethyl-benzoylamino)-benzamide,
N-(3-Bromo-benzyl)-3-(2-trifluoromethyl-benzoylamino)-benzamide,
N-(2,3-Dimethyl-benzyl)-3-(2-trifluoromethyl-benzoylamino)-benzamide,
N-(8-hydroxy-quinolin-2-yl)-3-(2-trifluoromethyl-benzoylamino)-benzamide,
N-(Benzyl)-3-(2-trifluoromethyl-benzoylamino)-benzamide,
N-(2-Methyl-benzyl)-3-(2-trifluoromethyl-benzoylamino)-benzamide,
N-(2-Chloro-benzyl)-3-(2-trifluoromethyl-benzoylamino)-benzamide,
N-(2-Methoxy-benzyl)-3-(2-trifluoromethyl-benzoylamino)-benzamide,
N-(2-Dimethylamino-benzyl)-3-(2-trifluoromethyl-benzoylamino)-benzamide,
N-(2-piperidin-1-yl-benzyl)-3-(2-trifluoromethyl-benzoylamino)-benzamide,
N-(2-Trifluoromethoxy-benzyl)-3-(2-trifluoromethyl-benzoylamino)-benzamide,
N-(2,3-dihydro-benzo[1,4]dioxin-5-ylmethyl)-3-(2-trifluoromethyl-benzoylamino)-benzamide,
N-(2-morpholin-4-yl-benzyl)-3-(2-trifluoromethyl-benzoylamino)-benzamide,
3-[2-(Trifluoromethyl)-benzoylamino]-N-(3-bromo-pyridine-2-yl)-benzamide,
3-[2-(Trifluoromethyl)-benzoylamino]-N-(5-methyl-pyridine-2-yl)-benzamide,
3-[2-(Trifluoromethyl)-benzoylamino]-N-(5-trifluoromethyl-pyridine-2-yl)-benzamide,
3-[2-(Trifluoromethyl)-benzoylamino]-N-(5-cyano-pyridine-2-yl)-benzamide,
3-[2-(Trifluoromethyl)-benzoylamino]-N-(5-bromo-pyridine-2-yl)-benzamide,
3-[2-(Trifluoromethyl)-benzoylamino]-N-(5-fluoro-pyridine-2-yl)-benzamide,
3-[2-(Trifluoromethyl)-benzoylamino]-N-(5-chloro-pyridine-2-yl)-benzamide,
3-[2-(Trifluoromethyl)-benzoylamino]-N-(5-iodo-pyridine-2-yl)-benzamide,
3-[2-(Trifluoromethyl)-benzoylamino]-N-(5-bromo-6-methyl-pyridine-2-yl)-benzamide,
3-[2-(trifluoromethyl)-benzoylamino]-N-1,3-benzothiazol-5-yl-benzamide,
3-[2-(trifluoromethyl)-benzoylamino]-N-(2-methyl-1,3-benzothiazol-6-yl)benzamide,
3-[2-(trifluoromethyl)-benzoylamino]-N-(2-methyl-1,3-benzoxazol-6-yl)-benzamide,
3-[2-(trifluoromethyl)-benzoylamino]-N-(5-chloro-1,3-benzoxazol-2-yl)benzamide,
3-[2-(trifluoromethyl)-benzoylamino]-N-(5-thien-3-ylpyridin-2-yl)benzamide,
3-[2-(trifluoromethyl)-benzoylamino]-N-(5-phenylpyridin-2-yl)benzamide, and
3-[2-(trifluoromethyl)-benzoylamino]-N-{5-[4-dimethylamino)phenyl]pyridin-2-yl}benzamide.

45. A compound according to claim 36, selected from
3-(2-Trifluoromethyl-benzoylamino)-N-quinolin-2-yl-benzamide,
N-(3-Cycloheptylcarbamoyl-phenyl)-2-trifluoromethylbenzamide,
N-[3-((S)-1-Naphthalen-1-yl-ethylcarbamoyl)-phenyl]-2-trifluoromethyl-benzamide,
N-[3-((R)-1-Naphthalen-1-yl-ethylcarbamoyl)-phenyl]-2-trifluoromethyl-benzamide,
N-{3-[(Naphthalen-1-ylmethyl)-carbamoyl]-phenyl}-2-trifluoromethyl-benzamide,
2-Trifluoromethyl-N-{3-[(2-trifluoromethyl-benzylamino)-methyl]-phenyl}-benzamide,
3-(2-Trifluoromethyl-benzoylamino)-N-(2-trifluoromethyl-benzyl)-benzamide,
N-(2-Bromo-benzyl)-3-(2-trifluoromethyl-benzoylamino)-benzamide,
N-(2-Phenyl-benzyl)-3-(2-trifluoromethyl-benzoylamino)-benzamide,
N-(3-Phenyl-benzyl)-3-(2-trifluoromethyl-benzoylamino)-benzamide,
N-(3-Bromo-benzyl)-3-(2-trifluoromethyl-benzoylamino)-benzamide,
N-(2,3-Dimethyl-benzyl)-3-(2-trifluoromethyl-benzoylamino)-benzamide,
N-(8-hydroxy-quinolin-2-yl)-3-(2-trifluoromethyl-benzoylamino)-benzamide,
N-(Benzyl)-3-(2-trifluoromethyl-benzoylamino)-benzamide,
N-(2-Methyl-benzyl)-3-(2-trifluoromethyl-benzoylamino)-benzamide,
N-(2-Chloro-benzyl)-3-(2-trifluoromethyl-benzoylamino)-benzamide,
N-(2-Methoxy-benzyl)-3-(2-trifluoromethyl-benzoylamino)-benzamide,
N-(2-Dimethylamino-benzyl)-3-(2-trifluoromethyl-benzoylamino)-benzamide,
N-(2-piperidin-1-yl-benzyl)-3-(2-trifluoromethyl-benzoylamino)-benzamide,
N-(2-Trifluoromethoxy-benzyl)-3-(2-trifluoromethyl-benzoylamino)-benzamide,
N-(2,3-dihydro-benzo[1,4]dioxin-5-ylmethyl)-3-(2-trifluoromethyl-benzoylamino)-benzamide,
N-(2-morpholin-4-yl-benzyl)-3-(2-trifluoromethyl-benzoylamino)-benzamide,
3-[2-(Trifluoromethyl)-benzoylamino]-N-(3-bromo-pyridine-2-yl)-benzamide,
3-[2-(Trifluoromethyl)-benzoylamino]-N-(5-methyl-pyridine-2-yl)-benzamide,
3-[2-(Trifluoromethyl)-benzoylamino]-N-(5-trifluoromethyl-pyridine-2-yl)-benzamide,
3-[2-(Trifluoromethyl)-benzoylamino]-N-(5-cyano-pyridine-2-yl)-benzamide,
3-[2-(Trifluoromethyl)-benzoylamino]-N-(5-bromo-pyridine-2-yl)-benzamide,
3-[2-(Trifluoromethyl)-benzoylamino]-N-(5-fluoro-pyridine-2-yl)-benzamide,
3-[2-(Trifluoromethyl)-benzoylamino]-N-(5-chloro-pyridine-2-yl)-benzamide,
3-[2-(Trifluoromethyl)-benzoylamino]-N-(5-iodo-pyridine-2-yl)-benzamide,
3-[2-(Trifluoromethyl)-benzoylamino]-N-(5-bromo-6-methyl-pyridine-2-yl)-benzamide,
3-[2-(trifluoromethyl)-benzoylamino]-N-1,3-benzothiazol-5-yl-benzamide,
3-[2-(trifluoromethyl)-benzoylamino]-N-(2-methyl-1,3-benzothiazol-6-yl)benzamide,
3-[2-(trifluoromethyl)-benzoylamino]-N-(2-methyl-1,3-benzoxazol-6-yl)benzamide,
3-[2-(trifluoromethyl)-benzoylamino]-N-(5-chloro-1,3-benzoxazol-2-yl)benzamide,
3-[2-(trifluoromethyl)-benzoylamino]-N-(5-thien-3-ylpyridin-2-yl)benzamide,
3-[2-(trifluoromethyl)-benzoylamino]-N-(5-phenylpyridin-2-yl)benzamide,
3-[2-(trifluoromethyl)-benzoylamino]-N-{5-[4-dimethylamino)phenyl]pyridin-2-yl}benzamide.

46. A compound according to claim 36, selected from
3-[2-(Trifluoromethyl)-benzoylamino]-N-(3-bromo-pyridine-2-yl)-benzamide,
3-[2-(Trifluoromethyl)-benzoylamino]-N-(5-methyl-pyridine-2-yl)-benzamide,
3-[2-(Trifluoromethyl)-benzoylamino]-N-(5-trifluoromethyl-pyridine-2-yl)-benzamide,
3-[2-(Trifluoromethyl)-benzoylamino]-N-(5-cyano-pyridine-2-yl)-benzamide,
3-[2-(Trifluoromethyl)-benzoylamino]-N-(5-bromo-pyridine-2-yl)-benzamide,
3-[2-(Trifluoromethyl)-benzoylamino]-N-(5-fluoro-pyridine-2-yl)-benzamide,
3-[2-(Trifluoromethyl)-benzoylamino]-N-(5-chloro-pyridine-2-yl)-benzamide,
3-[2-(Trifluoromethyl)-benzoylamino]-N-(5-iodo-pyridine-2-yl)-benzamide,
3-[2-(Trifluoromethyl)-benzoylamino]-N-(5-bromo-6-methyl-pyridine-2-yl)-benzamide, and
3-[2-(trifluoromethyl)-benzoylamino]-N-1,3-benzothiazol-5-yl-benzamide.

47. A pharmaceutical composition comprising a therapeutically effective amount of a compound according to claim 36, or a pharmaceutically acceptable salt thereof, and at least one pharmaceutically acceptable excipient.

48. A method of treatment of a disease selected from pain, cancer, inflammation and inflammatory diseases by administration of a therapeutically effective amount of a compound according to claim 36, or a pharmaceutically acceptable salt thereof, to a mammal in the need of such treatment.

49. A pharmaceutical composition comprising a compound of formula (II) 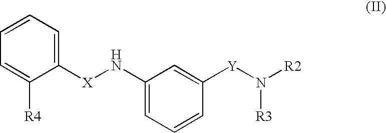
wherein
R² and R³ are independently selected from H and a moiety selected from alkyl, monocyclic or bicyclic carbocyclyl and monocyclic or bicyclic heterocyclyl, which moiety optionally is substituted with one or more R⁵;
R⁴ is selected from halogen, C1-C4-alkyl, monocyclic C5-C10-carbocyclyl, monocyclic 5 or 6-membered heterocyclyl, C1-C4-alkoxy, CF₃, NO₂, —C(O)O—C1-C4)-alkyl, and —OC(O)—C1-C4-alkyl;
each R⁵ is independently selected from halogen, alkyl, monocyclic or bicyclic carbocyclyl, monocyclic or bicyclic heterocyclyl, alkoxy, CN, CF₃, OH, NO₂, COOH, C(O)Oalkyl, OC(O)alkyl, wherein any cyclyl moiety may be substituted with one or more R⁶,
each R⁶ is independently selected from CF₃, halogen, alkyl, CF₃O, N,N-dialkylamino, phenyl, and saturated monocyclic heterocyclyl,
wherein in any of R², R³, R⁵ and R⁶, any cyclyl comprises 1-10 C and any alkyl and alkoxy comprises 1-6 C;
Y and X are independently selected from >CO and >CH₂;
or a pharmaceutically acceptable salt thereof, and at least one pharmaceutically acceptable excipient.

50. A composition according to claim 49, wherein R² and R³ are each independently selected from H and a moiety selected from C1-C6-alkyl, carbocyclyl and heterocyclyl, wherein any cyclyl is 5-7 membered monocyclic or is bicyclic having a 5- or 6-membered cycle fused with a 6-membered cycle.

51. A composition according to claim 49, wherein R² and R³ are each independently selected from H and a moiety selected from methyl, n-butyl, cyclohexyl, cycloheptyl, phenyl, pyridyl, quinolinyl, isoquinolinyl, benzimidazolyl, benzothiazolyl, 1H-indazolyl, indanyl and 1,2,3,4-tetrahydronaphthyl, which moiety is optionally substituted with one or more R⁵ as defined in claim 49.

52. A composition according to claim 49, wherein R³ is selected from H and a moiety selected from C1-C3 alkyl and monocyclic C5-C7 carbocyclyl, which moiety optionally is substituted with one or more moieties selected from monocyclic C5-C7 carbocyclyl.

53. A composition according to claim 49, wherein R³ is H.

54. A composition according to claim 49, wherein R⁴ is selected from CH₃O, CF₃, OC(O)CH₃, NO₂, C(O)OCH₃, COOH, F, Cl, Br; I, methyl, ethyl, cyclopentyl, phenyl and thienyl.

55. A composition according to claim 49, wherein X and Y are both >C═O

56. A composition according to claim 49, wherein each R⁵ is independently selected from halogen, alkyl, CN, CF₃, OH, monocyclic or bicyclic carbocyclyl and monocyclic or bicyclic heterocyclyl, wherein any cyclyl moiety may be substituted with one or more R⁶ as defined in claim 49.

57. A method of treatment of a disorder selected from fever, pain, cancer, inflammation and inflammatory diseases compound by administration to a mammal in need of such treatment, of a therapeutically effective amount of a compound of formula (II) 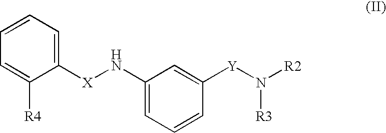
wherein
R² and R³ are independently selected from H and a moiety selected from alkyl, monocyclic or bicyclic carbocyclyl and monocyclic or bicyclic heterocyclyl, which moiety optionally is substituted with one or more R⁵;
R⁴ is selected from halogen, C1-C4-alkyl, monocyclic C5-C10-carbocyclyl, monocyclic 5 or 6-membered heterocyclyl, C1-C4-alkoxy, CF₃, NO₂, —C(O)O—C1-C4)-alkyl, and —OC(O)—C1-C4-alkyl;
each R⁵ is independently selected from halogen, alkyl, monocyclic or bicyclic carbocyclyl, monocyclic or bicyclic heterocyclyl, alkoxy, CN, CF₃, OH, NO₂, COOH, C(O)Oalkyl, OC(O)alkyl, wherein any cyclyl moiety may be substituted with one or more R⁶,
each R⁶ is independently selected from CF₃, halogen, alkyl, CF₃O, N,N-dialkylamino, phenyl, and saturated monocyclic heterocyclyl,
wherein in any of R², R³, R⁵ and R⁶, any cyclyl comprises 1-10 C and any alkyl and alkoxy comprises 1-6 C;
Y and X are independently selected from >CO and >CH₂;
or a pharmaceutically acceptable salt thereof.

58. A method of treatment of a disorder selected from fever, pain, cancer, inflammation and inflammatory diseases compound by administration to a mammal in need of such treatment, of a therapeutically effective amount of a compound of formula (I) 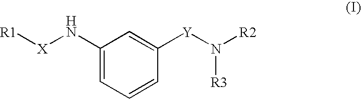
wherein
R¹ is selected from monocyclic or bicyclic carbocyclyl, monocyclic or bicyclic heterocyclyl, alkyl, and alkenyl, optionally substituted with one or more R⁴;
R² and R³ are each independently selected from H and a moiety selected from alkyl, monocyclic or bicyclic carbocyclyl and monocyclic or bicyclic heterocyclyl, which moiety optionally is substituted with one or more R⁵;
each R⁴ and R⁵ is independently selected from halogen, alkyl, monocyclic or bicyclic carbocyclyl, monocyclic or bicyclic heterocyclyl, alkoxy, CN, CF₃, OH, NO₂, C(O)Oalkyl, OC(O)alkyl, wherein any cyclyl moiety may be substituted with one or more R⁶,
each R⁶ is independently selected from CF₃, halogen, alkyl, CF₃O—, N,N-dialkylamino, phenyl, and saturated monocyclic heterocyclyl,
wherein in any of R¹-R⁶, any cyclyl comprises 1-10 C and any alkyl, alkenyl, and alkoxy comprises 1-6 C;
Y and X are independently selected from >CO and >CH₂;
or a pharmaceutically acceptable salt thereof.

59. The method according to claim 58, wherein R¹ is a moiety selected from phenyl, naphthyl, aromatic 5-6-membered heterocyclyl, cyclopropyl, methyl, ethyl and ethenyl, which moiety is optionally substituted with one or more R⁴ as defined in claim 58.

60. The method according to claim 58, wherein R² and R³ are independently selected from H and a moiety selected from C1-C4-alkyl, monocyclic or bicyclic carbocyclyl and monocyclic or bicyclic heterocyclyl, and C5-C7-cycloalkyl, optionally fused with phenyl; which moiety optionally is substituted with one or more R⁵ as defined in claim 58.

61. The method according to claim 58, wherein R³ is selected from H and a moiety selected from C1-C3 alkyl and monocyclic C5-C7 carbocyclyl, which moiety optionally is substituted with one or more moieties selected from monocyclic C5-C7 carbocyclyl.

62. The method according to claim 58, wherein R³ is H.

63. The method according to claim 58, wherein R⁴ is selected from halogen, C1-C4-alkyl, monocyclic C5-C10-carbocyclyl, monocyclic 5 or 6-membered heterocyclyl, C1-C4-alkoxy, CF₃, NO₂, COOH, —C(O)O—C1-C4)-alkyl, and —OC(O)—C1-C4-alkyl.

64. The method according to claim 58, wherein each R⁵ is independently selected from halogen, alkyl, CN, CF₃, OH, and monocyclic or bicyclic carbocyclyl and monocyclic or bicyclic heterocyclyl, wherein any cyclyl moiety may be substituted with one or more R⁶ as defined in claim 58.

65. The method according to claim 58, wherein X and Y are both >CO.

66. The method according to claim 58, wherein the compound is selected from
3-(2-chloro-benzoylamino)-N-benzyl-benzamide,
3-(2-chloro-benzoylamino)-N-(2-methyl-quinolin-4-yl)-benzamide,
3-(2-chloro-benzoylamino)-N-butyl-benzamide,
3-(2-chloro-benzoylamino)-N,N-dibenzyl-benzamide,
3-(2-chloro-benzoylamino)-N-(diphenylyl-methyl)-benzamide,
3-(2-chloro-benzoylamino)-N-(isoquinolin-5-yl-)-benzamide,
3-(2-chloro-benzoylamino)-N-(quinolin-8-yl-)-benzamide,
3-(2-chloro-benzoylamino)-N-(quinolin-2-yl-)-benzamide,
3-(2-chloro-benzoylamino)-N-(pyridine-2-ylmethyl)-benzamide,
3-(2-chloro-benzoylamino)-N-(pyridine-3-ylmethyl)-benzamide,
3-(2-chloro-benzoylamino)-N-(pyridine-2-yl)-benzamide,
3-(2-chloro-benzoylamino)-N-(1H-benzoimidazol-2-yl)-benzamide,
3-(2-chloro-benzoylamino)-N-(benzothiazol-6-yl)-benzamide,
3-(2-chloro-benzoylamino)-N-(1H-indazol-5-yl)-benzamide,
3-benzoylamino-N-quinolin-2-yl-benzamide,
3-acetylamino-N-quinolin-2-yl-benzamide,
thiophene-2-carboxylic acid [3-(quinolin-2-ylcarbamoyl)-phenyl]-amide,
furan-2-carboxylic acid [3-(quinolin-2-ylcarbamoyl)-phenyl]amide,
N-[3-(quinolin-2-ylcarbamoyl)-phenyl]-nicotinamide,
N-[3-(quinolin-2-ylcarbamoyl)-phenyl]-isonicotinamide,
3-phenylacetylamino-N-quinolin-2-yl-benzamide,
3-(cyclopropanecarbonyl-amino)-N-quinolin-2-yl-benzamide,
3-diphenylacetylamino-N-quinolin-2-yl-benzamide,
3-(3-cyclopentyl-propionylamino)-N-quinolin-2-yl-benzamide,
3-[(E)-(3-phenyl-acryloyl)amino]-N-quinolin-2-yl-benzamide,
3,4-dichloro-N-[3-(quinolin-2-ylcarbamoyl)-phenyl]-benzamide,
3-(3-chloro-benzoylamino)-N-quinolin-2-yl-benzamide,
3-(4-methoxy-benzoylamino)-N-quinolin-2-yl-benzamide,
3-(4-chloro-benzoylamino)-N-quinolin-2-yl-benzamide,
3-(3-trifluoromethyl-benzoylamino)-N-quinolin-2-yl-benzamide,
3-(2-chloro-benzoylamino)-N,N-dicyclohexyl-benzamide,
3-(2-chloro-benzoylamino)-N-indan-1-yl-benzamide,
3-(2-chloro-benzoylamino)-N-(1,2,3,4-tetrahydro-naphthalen-1-yl)-benzamide,
3-(2-chloro-benzoylamino)-N-cycloheptyl-benzamide,
3-(3,5-bis-trifluoromethyl-benzoylamino)-N-quinolin-2-yl-benzamide,
3-(2-bromo-benzoylamino)-N-quinolin-2-yl-benzamide,
3-(2-fluoro-benzoylamino)-N-quinolin-2-yl-benzamide,
3-(2,6-chloro-benzoylamino)-N-quinolin-2-yl-benzamide,
3-(2-methoxy-benzoylamino)-N-quinolin-2-yl-benzamide,
3-(2,6-dimethoxy-benzoylamino)-N-quinolin-2-yl-benzamide,
3-(2-trifluoromethyl-benzoylamino)-N-quinolin-2-yl-benzamide,
3-(2-methyl-benzoylamino)-N-quinolin-2-yl-benzamide,
3-(3,4,5-trimethoxy-benzoylamino)-N-quinolin-2-yl-benzamide,
3-(2-iodo-benzoylamino)-N-quinolin-2-yl-benzamide,
naphthalene-1-carboxylic acid [3-(quinolin-2-ylcarbamoyl)-phenyl]-amide,
naphthalene-2-carboxylic acid [3-(quinolin-2-ylcarbamoyl)-phenyl]amide,
3-(2-thiophene-2-yl-benzoylamino)-N-quinolin-2-yl-benzamide,
acetic acid 2-[3-(quinolin-2-ylcarbamoyl)-phenylcarbamoyl]-phenyl ester,
biphenyl-4-carboxylic acid [3-(quinolin-2-ylcarbamoyl)-phenyl]-amide,
3-(2-ethyl-benzoylamino)-N-quinolin-2-yl-benzamide,
3-(2-nitro-benzoylamino)-N-quinolin-2-yl-benzamide,
N-(3-cycloheptylcarbamoyl-phenyl)-3,5-bis-trifluoromethyl-2,6-dichloro-N-(3-cycloheptylcarbamoyl-phenyl)-benzamide,
N-(3-cycloheptylcarbamoyl-phenyl)-2-trifluoromethylbenzamide,
N-(3-cycloheptylcarbamoyl-phenyl)-2-methylbenzamide,
N-(3-cycloheptylcarbamoyl-phenyl)-3,4,5-trimethoxy-benzamide,
naphthalene-1-carboxylic acid (3-cycloheptylcarbamoyl-phenyl)-amide,
naphthalene-2-carboxylic acid (3-cycloheptylcarbamoyl-phenyl)-amide,
biphenyl-4-carboxylic acid (3-cycloheptylcarbamoyl-phenyl)-amide,
N-(3-cycloheptylcarbamoyl-phenyl)-2-thiophene-2-yl-benzamide,
N-(3-cycloheptylcarbamoyl-phenyl)-2-fluorobenzamide,
N-(3-cycloheptylcarbamoyl-phenyl)-2-methoxy-benzamide,
N-(3-cycloheptylcarbamoyl-phenyl)-2,6-dimethoxy-benzamide,
acetic acid 2-(3-cycloheptylcarbamoyl-phenylcarbamoyl)-phenyl ester,
N-(3-cycloheptylcarbamoyl-phenyl)-phthalamic acid methyl ester,
2-bromo-N-(3-cycloheptylcarbamoyl-phenyl)-benzamide,
N-(3-cycloheptylcarbamoyl-phenyl)-2-iodo-benzamide,
N-[3-((S)-1-naphthalen-1-yl-ethylcarbamoyl)-phenyl]-3,5-bis-trifluoromethyl-benzamide,
2-bromo-N-[3-((S)-1-naphthalen-1-yl-ethylcarbamoyl)-phenyl]-benzamide,
2-fluoro-N-[3-((S)-1-naphthalen-1-yl-ethylcarbamoyl)-phenyl]-benzamide,
2,6-dichloro-N-[3-((S)-1-naphthalen-1-yl-ethylcarbamoyl)-phenyl]-benzamide,
2-methoxy-N-[3-((S)-1-naphthalen-1-yl-ethylcarbamoyl)-phenyl]-benzamide,
2,6-dimethoxy-N-[3-((S)-1-naphthalen-1-yl-ethylcarbamoyl)-phenyl]-benzamide,
N-[3-((S)-1-naphthalen-1-yl-ethylcarbamoyl)-phenyl]-2-trifluoromethyl-benzamide,
2-methyl-N-[3-((S)-1-naphthalen-1-yl-ethylcarbamoyl)-phenyl]-benzamide,
2-iodo-N-[3-((S)-1-naphthalen-1-yl-ethylcarbamoyl)-phenyl]-benzamide,
naphthalene-1-carboxylic acid [3-((S)-1-naphthalen-1-yl-ethylcarbamoyl)-phenyl]-amide,
naphthalene-2-carboxylic acid [3-((S)-1-naphthalen-1-yl-ethylcarbamoyl)-phenyl]-amide,
biphenyl-4-carboxylic acid [3-((S)-1-naphthalen-1-yl-ethylcarbamoyl)-phenyl]amide,
N-[3-((S)-1-naphthalen-1-yl-ethylcarbamoyl)-phenyl]-2-thiophen-2-yl-benzamide,
3,4,5-trimethoxy-N-[3-((S)-1-naphthalen-1-yl-ethylcarbamoyl)-phenyl]-benzamide,
2-chloro-N-[3-((S)-1-naphthalen-1-yl-ethylcarbamoyl)-phenyl]-benzamide,
N-[3-((R)-1-naphthalen-1-yl-ethylcarbamoyl)-phenyl]-3,5-bis-trifluoromethyl-benzamide,
2-bromo-N-[3-((R)-1-naphthalen-1-yl-ethylcarbamoyl)-phenyl]-benzamide,
2-fluoro-N-[3-((R)-1-naphthalen-1-yl-ethylcarbamoyl)-phenyl]-benzamide,
2,6-dichloro-N-[3-((R)-1-naphthalen-1-yl-ethylcarbamoyl)-phenyl]-benzamide,
acetic acid 2-[3-((R)-1-naphthalen-1-yl-ethylcarbamoyl)-phenylcarbamoyl]-phenyl ester,
2-methoxy-N-[3-((R)-1-naphthalen-1-yl-ethylcarbamoyl)-phenyl]-benzamide,
2,6-dimethoxy-N-[3-((R)-1-naphthalen-1-yl-ethylcarbamoyl)-phenyl]-benzamide,
N-[3-((R)-1-naphthalen-1-yl-ethylcarbamoyl)-phenyl]-2-trifluoromethyl-benzamide,
2-methyl-N-[3-((R)-1-naphthalen-1-yl-ethylcarbamoyl)-phenyl]-benzamide,
3,4,5-trimethoxy-N-[3-((R)-1-naphthalen-1-yl-ethylcarbamoyl)-phenyl]-benzamide,
2-iodo-N-[3-((R)-1-naphthalen-1-yl-ethylcarbamoyl)-phenyl]-benzamide,
naphthalene-1-carboxylic acid [3-((R)-1-naphthalen-1-yl-ethylcarbamoyl)-phenyl]-amide,
naphthalene-2-carboxylic acid [3-((R)-1-naphthalen-1-yl-ethylcarbamoyl)-phenyl]-amide,
biphenyl-4-carboxylic acid [3-((R)-1-naphthalen-1-yl-ethylcarbamoyl)-phenyl]-amide,
N-[3-((R)-1-naphthalen-1-yl-ethylcarbamoyl)-phenyl]-2-thiophen-2-yl-benzamide,
2-chloro-N-[3-((R)-1-naphthalen-1-yl-ethylcarbamoyl)-phenyl]-benzamide,
N-{3-[(naphthalen-1-ylmethyl)-carbamoyl]-phenyl}-3,5-bis-trifluoromethyl-benzamide,
2-bromo-N-{3-[(naphthalen-1-ylmethyl)-carbamoyl]-phenyl}-benzamide,
2-fluoro-N-{3-[(naphthalen-1-ylmethyl)-carbamoyl]-phenyl}-benzamide,
2,6-dichloro-N-{3-[(naphthalen-1-ylmethyl)-carbamoyl]-phenyl}-benzamide,
acetic acid 2-{3-[(naphthalen-1-ylmethyl)-carbamoyl]-phenylcarbamoyl}-phenyl ester,
2-methoxy-N-{3-[(naphthalen-1-ylmethyl)-carbamoyl]-phenyl}-benzamide,
2,6-dimethoxy-N-{3-[(naphthalen-1-ylmethyl)-carbamoyl]-phenyl}-benzamide,
N-{3-[(naphthalen-1-ylmethyl)-carbamoyl]-phenyl}-2-trifluoromethyl-benzamide,
N-{3-[(naphthalen-1-ylmethyl)-carbamoyl]-phenyl}-2-methyl-benzamide,
3,4,5-trimethoxy-N-{3-[(naphthalen-1-ylmethyl)-carbamoyl]-phenyl}-benzamide,
2-iodo-N-{3-[(naphthalen-1-ylmethyl)-carbamoyl]-phenyl}-benzamide,
naphthalene-1-carboxylic acid {3-[(naphthalen-1-ylmethyl)-carbamoyl]-phenyl}-amide,
naphthalene-2-carboxylic acid {3-[(naphthalen-1-ylmethyl)-carbamoyl]-phenyl}-amide,
biphenyl-4-carboxylic acid {3-[(naphthalen-1-ylmethyl)-carbamoyl]-phenyl}-amide,
N-{3-[(naphthalen-1-ylmethyl)-carbamoyl]-phenyl}-2-thiopene-2-yl-benzamide,
2-chloro-N-{3-[(naphthalen-1-ylmethyl)-carbamoyl]-phenyl}-benzamide,
naphthalene-1-carboxylic acid [3-(2-chloro-phenylcarbamoyl)-phenyl]-amide,
3-(2-bromo-benzylamino)-N-naphthalen-1-ylmethyl-benzamide,
2-trifluoromethyl-N-{3-[(2-trifluoromethyl-benzylamino)-methyl]-phenyl}-benzamide,
3-(2-trifluoromethyl-benzoylamino)-N-(2-trifluoromethyl-benzyl)-benzamide,
N-(2-bromo-benzyl)-3-(2-trifluoromethyl-benzoylamino)-benzamide,
N-(2-phenyl-benzyl)-3-(2-trifluoromethyl-benzoylamino)-benzamide,
N-(3-phenyl-benzyl)-3-(2-trifluoromethyl-benzoylamino)-benzamide,
N-(3-bromo-benzyl)-3-(2-trifluoromethyl-benzoylamino)-benzamide,
N-(2,3-dimethyl-benzyl)-3-(2-trifluoromethyl-benzoylamino)-benzamide,
N-(8-hydroxy-quinolin-2-yl)-3-(2-trifluoromethyl-benzoylamino)-benzamide,
N-(benzyl)-3-(2-trifluoromethyl-benzoylamino)-benzamide,
N-(2-methyl-benzyl)-3-(2-trifluoromethyl-benzoylamino)-benzamide,
N-(2-chloro-benzyl)-3-(2-trifluoromethyl-benzoylamino)-benzamide,
N-(2-methoxy-benzyl)-3-(2-trifluoromethyl-benzoylamino)-benzamide,
N-(2-dimethylamino-benzyl)-3-(2-trifluoromethyl-benzoylamino)-benzamide,
N-(2-piperidin-1-yl-benzyl)-3-(2-trifluoromethyl-benzoylamino)-benzamide,
N-(2-trifluoromethoxy-benzyl)-3-(2-trifluoromethyl-benzoylamino)-benzamide,
N-(2,3-dihydro-benzo[1,4]dioxin-5-ylmethyl)-3-(2-trifluoromethyl-benzoylamino)-benzamide,
N-(2-morpholin-4-yl-benzyl)-3-(2-trifluoromethyl-benzoylamino)-benzamide,
3-[2-(trifluoromethyl)-benzoylamino]-N-(3-bromo-pyridine-2-yl)-benzamide,
3-[2-(trifluoromethyl)-benzoylamino]-N-(5-methyl-pyridine-2-yl)-benzamide,
3-[2-(trifluoromethyl)-benzoylamino]-N-(5-methyl-pyridine-2-yl)-benzamide,
3-[2-(trifluoromethyl)-benzoylamino]-N-(5-cyano-pyridine-2-yl)-benzamide,
3-[2-(Trifluoromethyl)-benzoylamino]-N-(3-bromo-pyridine-2-yl)-benzamide,
3-[2-(Trifluoromethyl)-benzoylamino]-N-(5-methyl-pyridine-2-yl)-benzamide,
3-[2-(Trifluoromethyl)-benzoylamino]-N-(5-methyl-pyridine-2-yl)-benzamide,
3-[2-(Trifluoromethyl)-benzoylamino]-N-(5-cyano-pyridine-2-yl)-benzamide,
3-[2-(Trifluoromethyl)-benzoylamino]-N-(5-trifluoromethyl-pyridine-2-yl)-benzamide,
3-[2-(Trifluoromethyl)-benzoylamino]-N-(5-bromo-pyridine-2-yl)-benzamide,
3-[2-(Trifluoromethyl)-benzoylamino]-N-(5-fluoro-pyridine-2-yl)-benzamide,
3-[2-(Trifluoromethyl)-benzoylamino]-N-(5-chloro-pyridine-2-yl)-benzamide,
3-[2-(Trifluoromethyl)-benzoylamino]-N-(5-iodo-pyridine-2-yl)-benzamide,
3-[2-(Trifluoromethyl)-benzoylamino]-N-(5-bromo-6-methyl-pyridine-2-yl)-benzamide,
3-[2-(trifluoromethyl)-benzoylamino]-N-1,3-benzothiazol-5-yl-benzamide,
3-[2-(trifluoromethyl)-benzoylamino]-N-(2-methyl-1,3-benzothiazol-6-yl)benzamide,
3-[2-(trifluoromethyl)-benzoylamino]-N-(2-methyl-1,3-benzoxazol-6-yl)benzamide,
3-[2-(trifluoromethyl)-benzoylamino]-N-(5-chloro-1,3-benzoxazol-2-yl)benzamide,
3-[2-(trifluoromethyl)-benzoylamino]-N-(5-thien-3-ylpyridin-2-yl)benzamide,
3-[2-(trifluoromethyl)-benzoylamino]-N-(5-phenylpyridin-2-yl)benzamide, and
3-[2-(trifluoromethyl)-benzoylamino]-N-{5-[4-dimethylamino)phenyl]pyridin-2-yl}benzamide.
